(19) United States
(12) Patent Application Publication
(10) Pub. No.: US 2026/0257061 A1
(43) Pub. Date: Sep. 3, 2026

(54) NEUROMUSCULAR STIMULATION SYSTEM AND METHOD TO TREAT GLOTTIC INSUFFICIENCY

(71) Applicant: The General Hospital Corporation, Boston, MA (US)

(72) Inventors: James T. Heaton, Boston, MA (US); James B. Kobler, Boston, MA (US); Steven M. Zeitels, Boston, MA (US); David M. Otten, Newton, MA (US)

(21) Appl. No.: 19/164,988

(22) PCT Filed: Mar. 14, 2024
(86) PCT No.: PCT/US2024/020015

§ 371 Date: Sep. 12, 2025

Related U.S. Application Data
(60) Provisional application No. 63/490,205, filed on Mar. 14, 2023.

(51) Int. Cl. A61N 1/36; A61N 1/05

(52) U.S. Cl. CPC A61N 1/36; A61N 1/0504

(57) ABSTRACT

Disclosed are systems and methods for physiological sensing and laryngeal stimulation. The system includes at least one sensor configured to be secured to a subject to record EMG signals of a subject and a processor coupled to the at least one sensor to receive the recorded EMG signals. The processor is also to evaluate the EMG signals, determine when the subject intends to activate a vocal fold, and control a generator to stimulate at least one vocal fold of the subject to treat glottic insufficiency, improve airway protection, or both. The system further includes an electrode configured to be secured to the subject to deliver the stimulation signal to an area of interest in the subject to stimulate the at least one vocal fold of the subject to treat glottic insufficiency, improve airway protection, or both.

### CROSS-REFERENCE TO RELATED APPLICATIONS

This application claims priority to U.S. Provisional Patent Application No. 63/490,205, filed on Mar. 14, 2023, which is incorporated herein by reference in its entirety.

### STATEMENT REGARDING FEDERALLY SPONSORED RESEARCH

Not Applicable

### BACKGROUND

The present disclosure relates generally to systems and methods for neuromuscular stimulation. More specifically, the present disclosure relates to systems and methods for stimulating vocal cord muscles to treat glottic insufficiency and/or improve airway protection.

Humans have a specialized organ at the intersection of the pharynx and trachea called the larynx or voice box. The larynx consists of multiple cartilages attached by joints, ligaments, and membranes that provide a skeletal framework for allowing air between the trachea and pharynx, nasal and oral passages. The primary cartilages of this framework include the cricoid cartilage forming at ring attached inferiorly to the trachea and superiorly to the thyroid cartilage, the shield-shaped thyroid cartilage which is open posteriorly and attaches inferiorly with the cricoid cartilage and superiorly to the hyoid bone, and the leaf-shaped epiglottic cartilage which attaches to the internal surface of the thyroid cartilage to cover the laryngeal inlet and protect the airway during swallowing. Further, the human larynx has a pair of true vocal folds (VF)s or cords aligned horizontally along an anterior-posterior axis that span between the thyroid cartilage anteriorly and the arytenoid cartilages posteriorly. These folds are normally the sound source for voice, and the VF closing and opening movements are called VF adduction and abduction, respectively, which enable the larynx to serve as a selective valve between the pharynx and trachea.

Normal voice production depends on adequate approximation, i.e., closure or adduction of the VFs for valving the tracheal air stream, adequate air pressure from the lungs and trachea to drive the VFs into vibration, appropriate tension of VF musculature to facilitate vibration, and adequate pliability of the VF surfaces to generate the inferior-to-superior wave of medial surface deformation during vibration, i.e., the mucosal wave. Incomplete tension or closure of the VFs during VF adduction is called glottic insufficiency, which can cause excessive air leakage during attempted phonation, resulting in a weak, breathy, and inefficient voice with easy fatiguability. Glottic insufficiency can also cause asymmetrical VF vibration, producing a rough or hoarse vocal quality, and turbulent air noises upon inspiration and expiration, i.e., stridor, due to airway obstruction by the flaccid VF or VFs. In addition, glottic insufficiency presents an increased risk of aspiration, the inappropriate passage of material, e.g., food, liquid, and/or secretions, through the glottis into the trachea.

The most common cause of glottic insufficiency is VF paralysis or paresis, which is a complete or partial loss of VF mobility, respectively. VF immobility can arise from a wide range of etiologies, including reactive tissue changes, tumor infiltration, cricoarytenoid joint fixation, or most often from disruption of intrinsic laryngeal muscle neural control. Unilateral VF paralysis or paresis (UVFP) is the most common cause of debilitating dysphonia, a voice disorder, and is usually accompanied by complaints of dysphagia, a swallowing disorder. Unilateral paralysis is often idiopathic but can accompany symptoms of an upper respiratory tract infection and is therefore presumed to stem from viral or bacterial infection of the ipsilateral recurrent laryngeal nerve (RLN) when the etiology is unknown. Trauma to one or both RLNs also commonly stems from iatrogenic lesions occurring during surgery of the thyroid gland, neck vasculature, trachea, esophagus, cervical spine, lungs, and heart.

Surgical treatment for UVFP to improve protective glottic closure and strengthen voice most commonly involves mechanical medialization, i.e., adduction, of the affected VF to a fixed position closer to the midline, thereby improving glottal closure and phonation. An initial intervention often involves an injection of biocompatible hydrogel or autologous fat to medialize the paralyzed VF, but such materials can succumb to resorption in weeks to months and are often followed by permanent laryngeal framework surgery if spontaneous reinnervation supporting phonation does not occur. Longer-lasting injectables such as hydroxylapatite are available to treat UVFP but can cause intense inflammation, and surgeons often avoid injecting materials that can cause long-term dysphonia if such materials are over-injected or poorly positioned.

An alternative but far less common surgical treatment for glottic insufficiency is laryngeal reinnervation, which is usually performed by transferring the ipsilateral ansa cervicalis, i.e., innervating superficial neck strap muscles, to the distal RLN via neurorrhaphy. Although some investigators have reported excellent VF position and voice results after ansa-to-RLN transfer, the technique has not been widely accepted. Laryngeal reinnervation has been found to provide voice outcomes comparable to mechanical medialization of the paralyzed VF, with younger patients achieving slightly better results via reinnervation and older patients doing better with mechanical medialization. However, ansa-to-RLN transfer involves a substantial delay of about 6-9 months for reinnervation to occur until voice outcome is known, and microsurgical neurorrhaphy after locating the respective nerves in the neck is more invasive and technically challenging than various means of mechanical medialization.

Thus, the primary surgical option for patients with symptomatic UVFP has been medialization laryngoplasty (ML) since its introduction in 1915. In this procedure, permanent repositioning of the paralyzed VF into a phonatory position can be achieved by placing an implant through a window cut in the thyroid cartilage lateral to the VF, thereby pushing it towards the glottic midline. However, about one-quarter of patients who undergo this procedure will still need revision surgery to reposition their implant or fine-tune their VF position. Recent efforts to improve ML, although rarely adopted, have used preoperative anatomical imaging to help guide thyroid lamina window placement, and to make custom implants before surgery rather than using a pre-formed design or taking the time to hand-carve implants intraoperatively.

In addition to ML, surgeons with advanced training and skill may also suture or otherwise affix the arytenoid cartilage in an adducted, phonatory position for improved VF tension and position. However, arytenoid adduction (AA) surgery can be technically challenging, potentially restricting access for many patients. In addition, AA closes more of the posterior glottis and can restrict the airway. As a result, about one-quarter of patients undergoing open-neck ML are not candidates for AA. Moreover, even good vocal outcomes after static VF repositioning typically may not restore a normal voice, and approximately one-third of new patients seeking treatment for dysphonia are diagnosed with UVFP. Accordingly, there is a need for improved treatment methods to restore dynamic VF adduction and address one or more of the challenges discussed above concerning conventional static treatments.

### SUMMARY

The present disclosure overcomes the aforementioned drawbacks by providing systems and methods for laryngeal functional electrical stimulation (LFES) of VF muscles to treat VF paralysis by improving VF adduction and abduction, i.e., closure and opening, respectively. Improved overall VF function can be exhibited in a variety of different ways, such as, e. g., restoring voice, enabling cough, and providing protective glottic closure when swallowing. In particular, the present disclosure provides systems and methods of using electrodes for LFES to restore glottic closure and improve voice and airway protection in cases of unilateral or bilateral VF paralysis. The LFES systems described herein detect the occurrence or intention of laryngeal contractions and electrically stimulate paralyzed muscles to achieve desired actions, such as adduction or abduction. In this way, the LFES systems and methods described herein can at least partially restore glottic closure to treat unilateral and/or bilateral VF paralysis.

In accordance with one aspect of the disclosure, a system is provided for physiological sensing and laryngeal stimulation. The system includes at least one sensor configured to be secured to a subject to record EMG signals of a subject and a processor coupled to the at least one sensor to receive the recorded EMG signals. The processor is also to stimulate at least one vocal fold of the subject to treat glottic insufficiency, improve airway protection, or both. The system further includes an electrode configured to be secured to the subject to deliver the stimulation signal to an area of interest in the subject to stimulate the at least one vocal fold of the subject to treat glottic insufficiency, improve airway protection, or both.

In accordance with another aspect of the disclosure, a system is provided for stimulating at least one vocal fold in a subject. The system includes at least one sensor for recording EMG signals of a subject and a processor coupled to the at least one sensor, the processor to determine if the EMG signals correspond to intended movement of the at least one vocal fold of the subject. The system further includes a generator to selectively generate a stimulation signal if the analyzed EMG signals are determined to correspond to the intended movement of the at least one vocal fold of the subject. The system further includes an electrode to provide the generated stimulation signal to a region of interest in the subject to elicit a movement of the at least one vocal fold of the subject corresponding to the intended movement of the at least one vocal fold of the subject.

In accordance with yet another aspect of the disclosure, a method of stimulating a larynx of a subject is provided. The method includes detecting, with at least one sensor coupled to the subject, EMG signals corresponding to intended contraction of at least one vocal fold of the subject, and analyzing the electromyography signals to determine a stimulation signal for contracting the at least one vocal fold. The method further includes delivering the stimulation signal to an area of interest in the subject to stimulate the at least one vocal fold of the subject to elicit a contraction of the at least one vocal fold of the subject corresponding to the intended contraction of the at least one vocal fold of the subject indicated in the EMG signals.

The foregoing and other aspects and advantages of the present disclosure will appear from the following description. In the description, reference is made to the accompanying drawings that form a part hereof, and in which one or more embodiments are shown by way of illustration. These embodiments do not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 is a flowchart depicting a Laryngeal Functional Electrical Stimulation (LFES) system to treat unilateral VF paralysis or paresis, in accordance with aspects of the present disclosure.

FIG. 2 is a block diagram of an example LFES system, in accordance with aspects of the present disclosure.

FIG. 3A is a schematic diagram of a processor of a transmitter of the LFES system of FIG. 2, in accordance with aspects of the present disclosure.

FIG. 3B is a schematic diagram of a coil interface of a transmitter of the LFES system of FIG. 2, in accordance with aspects of the present disclosure.

FIG. 4A is a schematic diagram of a coil interface of a receiver of the LFES system of FIG. 2, in accordance with aspects of the present disclosure.

FIG. 4B is a schematic diagram of a stimulation signal generator of a receiver of the LFES system of FIG. 2, in accordance with aspects of the present disclosure.

FIG. 5A is a plot illustrating an unprocessed surface electromyographic (EMG) signal recorded from a subject, in accordance with aspects of the present disclosure.

FIG. 5B is a plot illustrating an envelope of the EMG signal of FIG. 5A, in accordance with aspects of the present disclosure.

FIG. 5C is a plot illustrating the envelope of the EMG signal of FIG. 5B at a reduced time scale with a threshold voltage, in accordance with aspects of the present disclosure.

FIG. 6A is a schematic illustration of an example transmitter of the LFES system of FIG. 2, in accordance with aspects of the present disclosure.

FIG. 6B is a schematic illustration of an example receiver of the LFES system of FIG. 2, in accordance with aspects of the present disclosure.

FIG. 6C is a schematic illustration of the example receiver of FIG. 6B, including a silicone coating and electrodes, in accordance with aspects of the present disclosure.

FIG. 7A is a front view of an example electrode for use with the LFES system of FIG. 2, in accordance with aspects of the present disclosure.

FIG. 7B is a front view of another example electrode for use with the LFES system of FIG. 2, in accordance with aspects of the present disclosure.

FIG. 7C is a front view of yet another example electrode for use with the LFES system of FIG. 2, in accordance with aspects of the present disclosure.

FIG. 8 is a block diagram of another example LFES system, in accordance with aspects of the present disclosure.

FIG. 9 is a schematic diagram of an optical interface of a receiver of the LFES system of FIG. 8, in accordance with aspects of the present disclosure.

FIG. 10A is a schematic diagram of an optical interface of a transmitter of the LFES system of FIG. 8, in accordance with aspects of the present disclosure.

FIG. 10B is another schematic diagram of an optical interface of a transmitter of the LFES system of FIG. 8, in accordance with aspects of the present disclosure.

FIG. 11A is a top view of an example transmitter of the LFES system of FIG. 8, in accordance with aspects of the present disclosure.

FIG. 11B is a bottom view of the example transmitter of FIG. 11A, in accordance with aspects of the present disclosure.

FIG. 11C is a schematic illustration of an example receiver and battery pack of the LFES system of FIG. 8, in accordance with aspects of the present disclosure.

FIG. 11D is a schematic illustration of the LFES system of FIG. 8 in an assembled state in accordance with aspects of the present disclosure.

FIGS. 12A-12F are a series of plots illustrating the effects of using a blanking signal to reduce shock artifacts in a raw EMG signal using an LFES system, in accordance with aspects of the present disclosure.

FIG. 13A is a plot of an example stimulation pulse generated by an LFES system, in accordance with aspects of the present disclosure.

FIG. 13B is a plot of another example stimulation pulse generated by an LFES system in accordance with aspects of the present disclosure.

FIG. 14 is a flowchart of non-limiting example steps for a method of stimulating a subject's larynx, in accordance with aspects of the present disclosure.

FIG. 15A is a top view of an arrangement of an example electrode contacting a male cricothyroid (CT) muscle in accordance with aspects of the present disclosure.

FIG. 15B is a lateral view of the arrangement of FIG. 15A, in accordance with aspects of the present disclosure.

FIG. 15C is a top view of an arrangement of another example electrode contacting a male CT muscle, in accordance with aspects of the present disclosure.

FIG. 15D is a lateral view of the arrangement of FIG. 15C, in accordance with aspects of the present disclosure.

FIG. 15E is a top view of an arrangement of an example electrode contacting a female CT muscle, in accordance with aspects of the present disclosure.

FIG. 15F is a lateral view of the arrangement of FIG. 15E, in accordance with aspects of the present disclosure.

FIG. 15G is a top view of an arrangement of still another example electrode contacting a female CT muscle, in accordance with aspects of the present disclosure.

FIG. 15H is a lateral view of the arrangement of FIG. 15G, in accordance with aspects of the present disclosure.

FIG. 16A is a lateral left side view of a canine model trachea, in accordance with aspects of the present disclosure.

FIG. 16B is a lateral right side view of the canine model trachea of FIG. 16A, in accordance with aspects of the present disclosure.

FIG. 17A-17C are a series of schematic illustrations of a method of bluntly dissecting a canine model larynx left VF and inserting an electrode of an LFES system therein, in accordance with aspects of the present disclosure.

FIG. 17D is a cross-sectional view of a canine model larynx left VF taken through a coronal plane, in accordance with aspects of the present disclosure.

FIGS. 18A-18D are a series of schematic illustrations of right VF stimulation in a canine model using LFES, in accordance with aspects of the present disclosure.

FIG. 19 is a plot of different signals recorded during a closed-loop stimulation of phonation in a canine model, in accordance with aspects of the present disclosure.

FIG. 20 is a plot of average phonation onset (i.e., activation) and offset (i.e., termination) latencies recorded during a closed-loop stimulation of phonation in a canine model, in accordance with aspects of the present disclosure.

FIGS. 21A-21F are a series of schematic illustrations of a method of bluntly dissecting a cadaveric larynx to create an insertion path for an example electrode of an LFES system, in accordance with aspects of the present disclosure.

FIGS. 22A-22F are a series of schematic illustrations of a method of inserting an electrode of an LFES into the cadaveric larynx of FIGS. 21A-21F, in accordance with aspects of the present disclosure.

FIGS. 23A-23F are a series of schematic illustrations that are similar to those of FIGS. 22A-22F, but depicting a cadaveric larynx with a superior third of a right side of thyroid cartilage having been removed, in accordance to aspects of the present disclosure.

FIGS. 24A-24F are a series of schematic illustrations that are similar to those of FIGS. 23A-23F, but depicting a cadaveric larynx with the thyroid cartilage having been detached at a cricothyroid joint, in accordance to aspects of the present disclosure.

FIGS. 25A-25F are a series of schematic illustrations that are similar to those of FIGS. 24A-24F, but depicting a cadaveric larynx with the thyroid cartilage having been completely removed, in accordance to aspects of the present disclosure.

FIGS. 26A-26F are a series of schematic illustrations that are similar to those of FIGS. 25A-25F, but depicting another example electrode of an LFES system, in accordance with aspects of the present disclosure.

FIG. 27A is a schematic illustration of a receiver of an inductive laryngeal stimulation (ILS) system, in accordance with aspects of the present disclosure.

FIG. 27B is a schematic illustration of implantation of the receiver of FIG. 27A in a canine model, in accordance with aspects of the present disclosure.

FIG. 27C is a schematic illustration of an X-ray image of the receiver implanted in the canine model of FIG. 27B, in accordance with aspects of the present disclosure.

FIG. 27D is a schematic illustration of a transmitter of the ILS system positioned external to the canine model of FIG. 27B, in accordance with aspects of the present disclosure.

FIG. 28A is a schematic illustration of a nerve cuff electrode's electrical connection port implanted near a left shoulder of a canine model with the nerve cuff contacting a left neck motor nerve, in accordance with aspects of the present disclosure.

FIG. 28B is a schematic illustration of a nerve cuff electrode implanted near a neck midline of a canine model with the nerve cuff contacting the left RLN, in accordance with aspects of the present disclosure.

FIG. 29 is a schematic illustration of a cylindrical intramuscular electrode, in accordance with aspects of the present disclosure.

FIG. 30A is a schematic illustration of a method of implanting the electrode of FIG. 29 in a canine model, in accordance with aspects of the present disclosure.

FIG. 30B is a schematic illustration of cylindrical electrodes implanted in the canine model of FIG. 30A, in accordance with aspects of the present disclosure.

FIG. 30C is a schematic illustration of an X-ray image of the electrodes implanted in the canine model of FIG. 30B, in accordance with aspects of the present disclosure.

FIG. 30D is a schematic illustration of an electrode anchor implanted in a right-side VF of the canine model of FIG. 30B, in accordance with aspects of the present disclosure.

FIG. 30E is a schematic illustration of an electrode anchor implanted in a left-side VF of the canine model of FIG. 30B, in accordance with aspects of the present disclosure.

FIG. 31 is a schematic diagram of an example experimental setup for performing closed-loop stimulation of laryngeal muscles in a canine model, in accordance with aspects of the present disclosure.

FIG. 32A is a schematic illustration of VFs of an example canine model in a rest position, in accordance with aspects of the present disclosure.

FIG. 32B is a schematic illustration of the VFs of FIG. 32A in a left-side adduction stimulation position, in accordance with aspects of the present disclosure.

FIG. 32C is a schematic illustration of VFs of another example canine model in a rest position, in accordance with aspects of the present disclosure.

FIG. 32D is a schematic illustration of the VFs of FIG. 32C in a right-side adduction stimulation position, in accordance with aspects of the present disclosure.

FIGS. 33A-33F are a series of schematic illustrations of VFs of a canine model undergoing VF stimulation using a variety of different wire entry points, in accordance with aspects of the present disclosure.

FIGS. 33G-33L are a series of schematic illustrations of high-speed video frames of VFs of a canine model across a single vibration cycle during phonation, in accordance with aspects of the present disclosure.

FIGS. 34A-34C are a series of schematic illustrations of VFs of a canine model undergoing VF stimulation of the CT muscle producing frequency-modulated phonation, in accordance with aspects of the present disclosure.

FIG. 35 is a plot of different signals recorded during a closed-loop stimulation of frequency-modulated phonation of FIG. 34A-34C in a canine model using an LFES, in accordance with aspects of the present disclosure.

FIG. 36 is a schematic illustration of another example experimental setup for performing closed-loop stimulation of laryngeal muscles in a canine model, in accordance with aspects of the present disclosure.

FIG. 37A is a schematic illustration of VFs of the canine model of FIG. 36 in an abducted position, in accordance with aspects of the present disclosure.

FIG. 37B is a schematic illustration of a VFs of the canine model of FIG. 37A in an adducted position, in accordance with aspects of the present disclosure.

FIGS. 38A-38D are a series of schematic illustrations of VFs of a canine model undergoing stimulation with a battery-powered stimulation (BLS) system, in accordance with aspects of the present disclosure.

### DESCRIPTION

Before any aspects of the disclosure are explained in detail, it is to be understood that the present disclosure is not limited in its application to the details of construction and the arrangement of components set forth in the following description or illustrated in the following drawings. The present disclosure is readily extended to other aspects and implementations and may be practiced or carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a controller device, a process being executed (or executable) by a controller device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other controller devices, or may be included within another component (or system, module, and so on).

In the methods described herein, the steps can be carried out in any order without departing from the principles of the disclosure, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “substantially” as used herein refers to a majority of, or mostly, as in at least about 50%, at least about 60%, at least about 70%, at least about 80%, at least about 90%, at least about 95%, at least about 96%, at least about 97%, at least about 98%, at least about 99%, at least about 99.5%, at least about 99.9%, at least about 99.99%, or at least about 99.999% or more.

The following discussion is presented to enable a person skilled in the art to make and use aspects of the disclosure. Various modifications to the illustrated configurations or processes will be readily apparent to those skilled in the art, and the generic principles herein can be applied to other aspects and applications within the scope of the present disclosure and the understanding of one of skill based thereon. Thus, the present disclosure is not intended to be limited to particular embodiments or aspects shown, but is to be accorded the widest scope consistent with the principles and features disclosed herein. The following detailed description is to be read with reference to the figures, in which like components or elements in different figures have like reference numerals. The figures, which are not necessarily to scale, depict selected aspects and configurations or processes and are not intended to limit the scope of the disclosure. Skilled artisans will recognize that the examples provided herein have many useful alternatives and fall within the scope of the disclosure.

In accordance with aspects of the present disclosure, mechanisms (which can, for example, include systems, methods, and media) for using laryngeal stimulation systems to treat glottic insufficiency via neuromuscular stimulation are provided.

Generally, the present disclosure provides systems, methods, and media for using a laryngeal stimulation device that can advantageously detect and record EMG signals of a subject corresponding to intended VF movement, generate a stimulation signal based on an analysis of the recorded EMG signals, and stimulate the VF(s) of the subject using the stimulation signal. Thus, aspects of the present disclosure provide systems, methods and media for stimulating one or both VFs of a subject to enhance and/or restore VF function to treat glottic insufficiency.

In some non-limiting examples, a laryngeal stimulation device may be configured as a closed-loop stimulation system that includes one or more sensors and one or more electrodes. The closed-loop stimulation system may be capable of generating a stimulation system based on EMG signals of a subject that are recorded, the EMG signals corresponding to an intended movement of the subject's VF(s). In this way, a subject with glottic insufficiency, which is commonly caused by VF paralysis or paresis, can experience enhanced dynamic VF function that resembles intended or normal VF function. Moreover, enhancing dynamic VF function can improve phonation, airway protection, and VF movement symmetry. In some aspects, the sensor(s) are capable of detecting and recording EMG signals from a patient, such as EMG signals corresponding to a functional VF in the case of UVFP. The recorded EMG signals can then be analyzed to generate a stimulation signal corresponding to the intended movement of an impaired VF, or impaired VFs. Further, the stimulation signal can be transmitted to the paralyzed VF via one or more contact members, such as electrodes surgically implanted within the subject's neck, e.g., between the subject's thyroarytenoid muscle and lateral cricoarytenoid muscle. In this way, the laryngeal stimulation device can provide a closed-loop stimulation system in which the physiological events in the subject, e.g., functional VF movement or neck muscle movement, trigger a stimulation signal to restore function of an impaired VF in real time.

Further, use of a closed-loop LFES system can greatly reduce power requirements of a stimulation device, as stimulation signals are only generated upon intended VF movement, in addition to being better suited to respond to biological or functional needs of the subject in comparison to preset stimulation generation patterns. That is, the closed-loop system uses the subject's own physiological markers to generate a stimulation signal in real time and emulate normal VF function. Moreover, biological functions reliant on VF function such as coughing, speaking, and swallowing are intermittent rather than rhythmic, and are therefore well-suited to a closed loop control approach. Therefore, the closed-loop FES systems discussed herein can be used to effectively treat VF paralysis or paresis in subjects with glottic insufficiency by maximizing a return to normal VF function.

In particular, an LFES system may include a transmitter that is configured to wirelessly instruct a receiver to generate a stimulation signal upon detecting and analyzing a subject's EMG associated with the intended VF function. In some aspects, the transmitter is located external to a subject, while the receiver is implanted within the subject, e.g., within the subject's neck or adjacent body location, to provide chronic stimulation therapy. The transmitter and/or receiver can be coupled to a monitoring or control system which can be configured to control stimulation signal generation and optimize performance of the LFES system. For example, the control system can set thresholds for stimulation signal generation and/or termination to provide hysteresis during EMG detection process, which in turn can improve the stability of the generated stimulation signal(s). Therefore, using a closed-loop laryngeal stimulation system in accordance with aspects of the present disclosure can enhance and/or restore VF function, thereby improving patient safety and satisfaction.

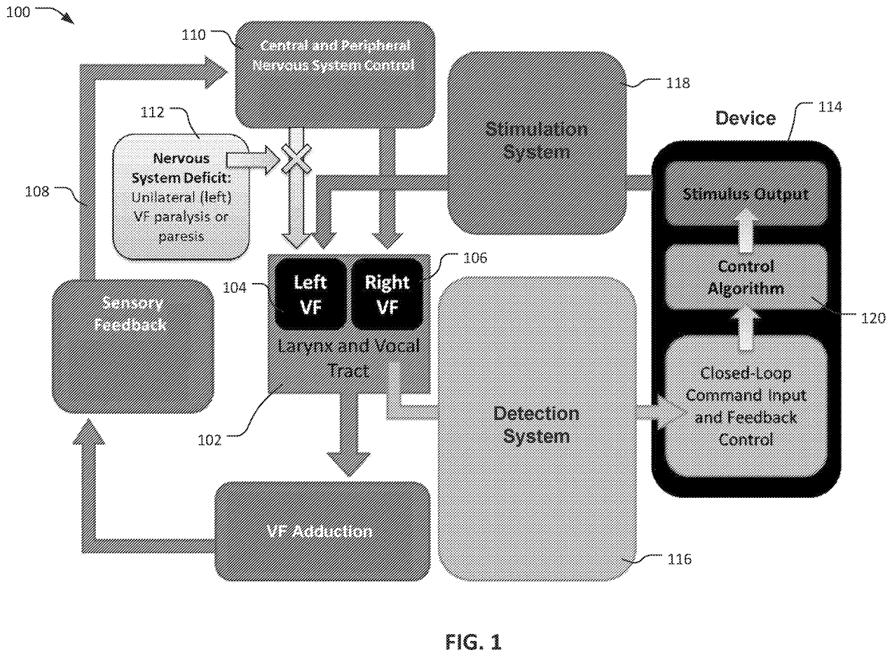

FIG. 1 illustrates a closed-loop stimulation system 100 to treat paralyzed or paretic VF muscles in a subject. As discussed above, the human larynx includes a pair of VFs that are normally the sound source for voice and that enable the larynx to serve as a selective valve between the pharynx and the trachea. Specifically, VFs are normally abducted and adducted, i.e., opened and closed, to selectively allow air through the trachea. However, impairment of one or both of the VFs can lead to a variety of harmful effects, including glottic insufficiency, i.e., reduced phonation, stridor, and/or aspiration. One common cause of VF impairment is a deficit within a subject's nervous system. FIG. 1 illustrates an example of such a deficit within a subject's larynx 102, i.e., vocal tract, that can include a left VF 104 and a right VF 106. Normally, the VFs 104, 106 are bilaterally adducted to perform one or more normal biological functions, such as phonation, cough, Valsalva, and/or airway protection. Adduction of the VFs 104, 106 forms a portion of a sensory feedback loop 108, which can also include a variety of normal biological feedback functions, e.g., acoustic feedback, somasthesia, and/or proprioception. The sensory feedback loop 108 can also include the subject's nervous system 110, e.g., a subject's central nervous system (CNS) and/or peripheral nervous system (PNS), which in turn can provide electrical signals to the muscles surrounding the VFs 104, 106. In particular, the nervous system 110 can provide electrical reflex and/or voluntary laryngeal adduction signals to the larynx 102 and the VFs 104, 106, thus closing the sensory feedback loop 108. However, a nervous system deficit 112 can prevent the normal electrical signals from reaching one or both of the VFs 104, 106, thereby causing unilateral or bilateral VF paralysis or paresis. For example, the nervous system deficit 112 may prevent a normal electrical signal from reaching the left VF 104, thus preventing the left VF 104 from being normally adducted and resulting in UVFP.

To treat UVFP, the closed-loop stimulation system 100 can provide a stimulation signal to the left VF 104 that, for example, may replace or resemble the electrical signal that normally is provided to the left VF 104 from the nervous system 110. To accomplish this, the closed-loop stimulation system 100 can include a device 114 configured to detect bioelectric signals, generate a stimulation signal corresponding to the bioelectric signals, and provide the generated stimulation signal to the left VF 104. In some aspects, the device 114 can include a detection system 116 with one or more sensors that are configured to detect and record biosignals corresponding to the intended VF function. For example, the detection system 116 can include a plurality of electrical contacts to detect bioelectric actional potentials corresponding to VF function, e.g., EMG signals in the larynx 102, EMG signals of the functional right VF 106, EMG of extra-laryngeal muscles associated with laryngeal function, and/or electroneuronography (ENOG) signals of cranial or spinal nerves. In other examples, the detection system 116 may include movement sensors, e. g., magnetic field sensors, pressure sensors, strain or bend gauge sensors, accelerometers, bioimpedance sensors, gyroscopes, displacement sensors, microphones, central and peripheral nervous system recordings, etc., to detect laryngeal movement or intended VF function.

Once the detection system 116 has recorded biosignals corresponding to the intended VF function, the resulting signals can be provided to a stimulation system 118 of the device 114 that can be configured to generate and provide a stimulation signal to the impaired left VF 104. For example, the stimulation system 118 can include a control system 120 that generates the stimulation signal to resemble the bioelectric potential that would normally cause adduction of the left VF 104. The generated stimulation signal can then be provided to the larynx 102 and the left VF 104 using, for example, a plurality of electrodes that are coupled to the left VF 104 and/or the extra-laryngeal muscles associated with the left VF 104. Thus, the device 114 can improve adduction of the left VF 104 after being triggered by bioelectric potentials or laryngeal movements, respiratory movements, other body movements, or manually activated switches correlated with the intended VF function, meaning that the closed-loop stimulation system 100 can stimulate the left VF 104 with a stimulation signal that is generated from the subject's own physiological activity. In this way, stimulated VF function can more closely resemble normal, intended VF function, which in turn can increase subject comfort, safety, and satisfaction.

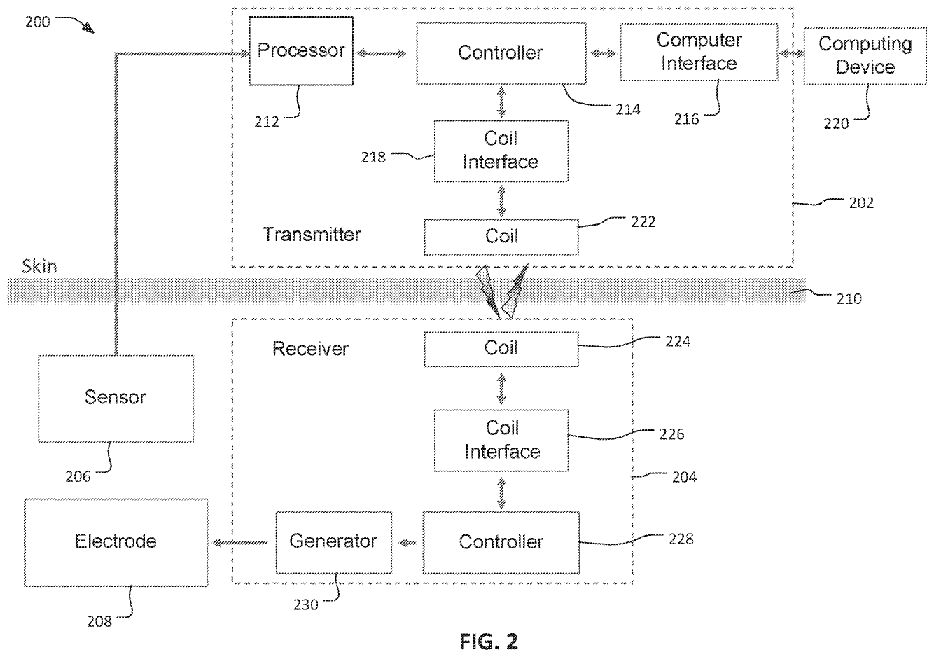

Referring now to the non-limiting example illustrated in FIG. 2, a closed-loop LFES system (e.g., an inductively-powered LFES system 200) can be used to restore and/or enhance a subject's VF function by detecting a subject's biosignals associated with normal VF function and stimulate an impaired function with a stimulation signal. In some aspects, an LFES system can include at least one sensor to detect or record the EMG signals of a subject, a processor to generate a stimulation signal after analyzing the EMG signal, and an electrode to deliver the stimulation signal to the patient. More specifically, an LFES system can include a first device or transmitter that can be inductively coupled with a second device or receiver without requiring any physical attachment therebetween. Specifically, the transmitter can be coupled to at least one sensor which can detect EMG signals of the subject corresponding to normal VF function. The transmitter can process the EMG signals via a control process and then selectively transmit an activation signal to the receiver to generate a stimulation signal. Further, the receiver can be coupled to at least one electrode that is implanted within or proximal to a subject's VF(s), and the receiver can output the stimulation signal to the VF(s) via the electrode(s).

In some aspects, an LFES system 200 can include a transmitter 202, a receiver 204, at least one sensor 206, and at least one electrode 208. The transmitter 202 and the receiver 204 can be provided within a single device, or the transmitter 202 and the receiver 204 can be provided as separate devices that are electrically coupled to one another. In some aspects, the receiver 204 can be configured to be implanted within a subject, e.g., within a subject's neck or adjacent body location for the electrode 208 to reach the subject's VF(s), whereas the transmitter 202 can be configured as an external device. That is, the receiver 204 can be surgically implanted under a subject's skin 210, while the transmitter 202 can be arranged external to the subject's skin 210. Specific implantation regions are discussed in the Examples below. The transmitter 202 can be configured to control the receiver 204 to generate and provide the stimulation signal to a region of interest in the subject via the plurality of electrodes 208, as will be discussed below in greater detail.

Further, the transmitter 202 can be coupled to the at least one sensor 206 and/or a plurality of sensors 206, and the plurality of sensors 206 may also be surgically implanted within a subject's larynx and/or VF(s). It is contemplated that any suitable sensor may be used for the plurality of sensors 206, such as, e.g., EMG recording electrodes, magnetic field sensors, pressure sensors, strain or bend gauge sensors, accelerometers, bioimpedance sensors, gyroscopes, displacement sensors, microphones, central and peripheral nervous system recordings, etc. Accordingly, the plurality of sensors 206 can be configured to detect the intended movement of the subject's larynx and/or intended VF function, other larynx-correlated body movements, or manually activated switches. As discussed above, the plurality of sensors 206 can be coupled to the transmitter 202, meaning that the transmitter 202 can receive the signals recorded by the plurality of sensors 206. For example, in the case of UVFP, the plurality of sensors 206 can record an EMG signal of a subject's functional VF, and the EMG signal can be provided to the transmitter 202 for processing.

Moreover, the transmitter 202 can include a variety of internal components, such as a first processor 212, a first controller 214, a computer interface 216, and a first coil interface 218. In some aspects, the first processor 212 is an instrumentation amplifier that processes an EMG signal recorded by the plurality of sensors 206. For example, the first processor 212 can include a variety of filtering components to remove any common-mode noise from the EMG signal and amplify the resulting signal by a gain, as will be discussed below in greater detail. The first processor 212 can also generate a stimulation signal that is configured to stimulate one or both VFs of a subject, e.g., a stimulation signal to elicit a contraction of one or both VFs, or the LFES 200 can include additional circuitry to generate the stimulation signal, as discussed below. Further, it will be understood that the first processor 212 can be included as part of the first controller 414, or vice versa, in some aspects. The first controller 214 can be configured to control the operation of the transmitter 202, including processing the EMG signal and interfacing with the receiver 204 and/or an external computing device 220. Specifically, the computer interface 216 of the transmitter 202 can be configured as a universal asynchronous receiver-transmitter (UART), a Bluetooth interface, a universal serial bus (USB) interface, or another type of interface. In some aspects, the computing device 220 serves as a power source for the LFES system 200, e.g., through the computer interface 216, and the computing device 220 can be used to automatically or manually, e.g., by a human operator, monitor and control operation of the LFES system 200.

Further, the computing device 220 can include a variety of software-implemented control processes to regulate stimulation signal generation and output to ensure subject safety. Specifically, the computing device 220 can be in communication with the first controller 214 to execute or run a control process which evaluates if the EMG signal detected by the plurality of sensors 206 is within a predetermined threshold voltage range, and the first controller 214 can regulate stimulation signal generation based on the result of the evaluation by the computing device 220 to ensure stimulation signal stability. In some examples, the computing device 220 can set a first or activation voltage threshold and a second or termination voltage threshold, as will be discussed below in greater detail.

The computing device 220 can further instruct the first controller 214 to interface with the receiver 204 to generate a stimulation signal. Specifically, the transmitter 202 and the receiver 204 can be in electrical communication with one another via a wireless communication interface, e.g., an inductive interface, an optical interface, a radio frequency (RF) interface, or any combination thereof. For example, a wireless communication interface can comprise the first coil interface 218 of the transmitter 202 and a second coil interface 226 of the receiver 204. The first coil interface 218 can include a first coil 222, e.g., an inductive coil, that can be placed in inductive communication with a second coil 224 of the second coil interface 226. In some aspects, power and/or information about a detected EMG signal and a desired stimulation signal, is communicated across the coil interfaces 218, 226, that is, between the transmitter 202 and the receiver 204. Thus, it will be understood that the receiver 204 may not require a separate power source, as the transmitter 202 can wirelessly provide power to the receiver 204 via the coil interfaces 218, 226. In some aspects, communication between the transmitter 202 and the receiver 204 is provided by making slight variations to the frequency used to transmit power to the receiver 204 via the coil interfaces 218, 226. The second coil interface 226 can include circuitry to detect and measure these frequency variations, which can then be transformed into instructions to generate and output a stimulation signal, as discussed below.

With continued reference to FIG. 2, the receiver 204 can include a second controller 228 and a stimulation signal generator 230, the second controller 228 coupled to the second coil interface 226 and the stimulation signal generator 230. The second controller 228 can be configured to control the operation of the receiver 204, including receiving instructions, e. g., instructions provided in an activation signal, from the first controller 214 of the transmitter 202 via the second coil interface 226. The second controller 228 can also be configured to cause the stimulation signal generator 230 to generate a stimulation signal. In some aspects, the stimulation signal generator 230 is an amplifier and/or a digital-to-analog (D/A) converter, as will be discussed below in greater detail. Alternatively, the first processor 212 can operate the stimulation signal generator to generate a stimulation signal, as discussed above. Further, the stimulation signal can correspond to the intended function of a subject's impaired VF, which can be determined using EMG signals of the subject's normally functioning VF and/or extra-laryngeal muscles, or other physiological or mechanical signals correlated with intended VF function. In some aspects, the stimulation signal can be generated as a pulse with a frequency that is controlled by the second controller 228 and/or the transmitter 202. After the stimulation signal is generated, the second controller 228 can instruct the stimulation signal generator 230 to the plurality of electrodes 208, thereby stimulating a region of interest, e.g., an impaired VF, of the subject.

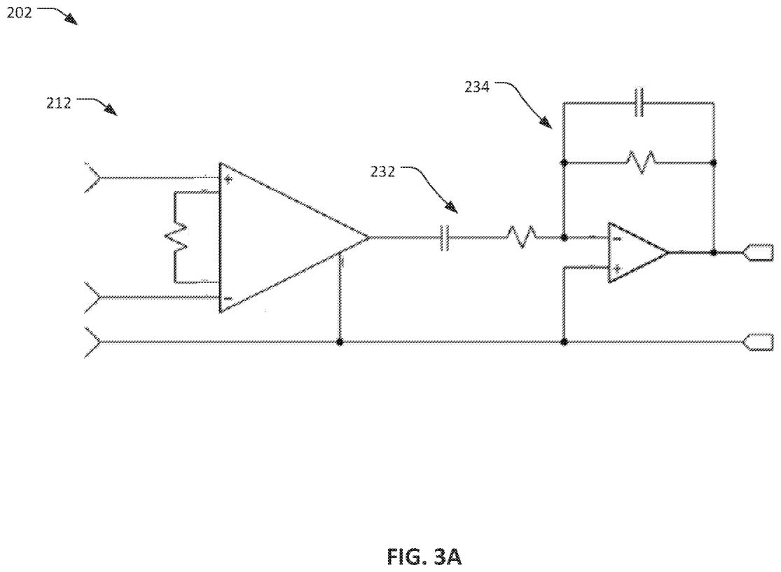

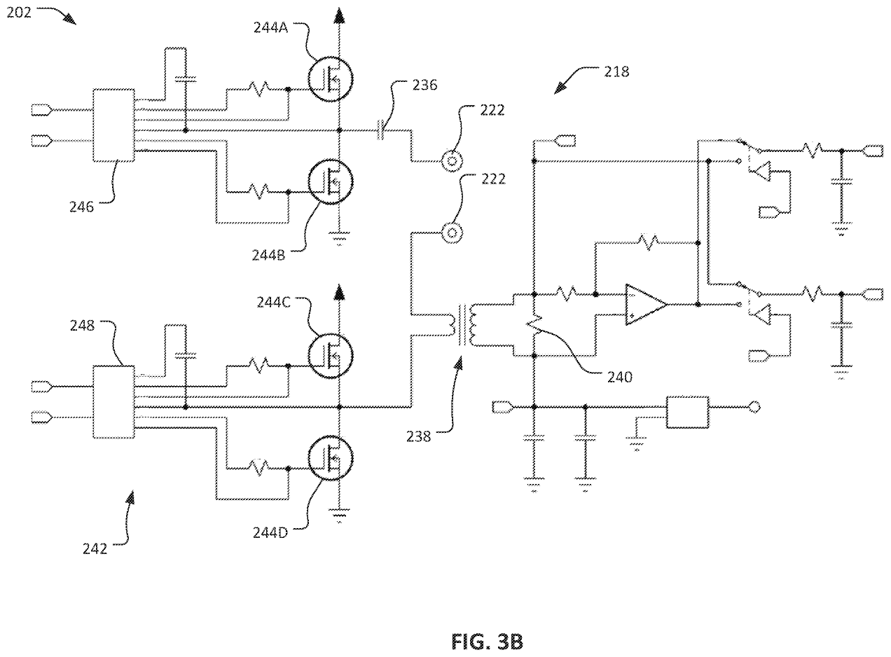

Referring now to FIGS. 3A and 3B, schematic diagrams are illustrated of the components of the transmitter 202. As discussed above, the transmitter 202 can process an incoming raw EMG signal, e.g., an EMG signal obtained by the plurality of sensors 206 with the first processor 212. In the non-limiting example illustrated in FIG. 3A, the first processor 212 can include a variety of filtering components, including, for example, artifact-rejecting switches, a high-pass filter, a low-pass filter, and/or a band pass filter. In some examples, the first processor 212 can include a high-pass filter 232 with a first cutoff frequency, which can be between about 1 hertz (Hz) and about 100 Hz, or between about 1 Hz and about 10 Hz, or about 10 Hz. The first processor 212 can further include a low-pass filter 234 with a second cutoff frequency, which can be between about 100 Hz and about 10 kilohertz (kHz), or between about 100 Hz and about 1 kHz, or about 1 kHz. In addition, the first processor 212 can amplify the processed EMG signal by a gain of about 1000.

Referring now to FIG. 3B, and as discussed above, the transmitter 202 can be configured to provide inductively coupled power and instructions to the receiver 204 (see FIG. 2) via the first coil interface 218. To accomplish this, the first coil interface 218 may be implemented as an air-core transformer in which a primary coil, e.g., the first coil 222, is located on the transmitter 202 and a secondary coil, e.g., the second coil 224, is located on the receiver 204 (see FIG. 2). In some examples, the first coil 222 includes between 24 and 72 turns, or about 36 turns arranged as nine turns on each of four layers of the transmitter 202. The first coil 222 can be coupled between, i.e., in series with, a first capacitor 236 and a current-sensing circuit 238. It is contemplated that the value of the first capacitor 236 can be selected to create a resonant circuit with the inductance of the first coil 222, such that the value of the first capacitor 236 can be between about 1 nanofarad (nF) and about 100 nF, or about 10 nF. Further, the current-sensing circuit 238 can be configured to measure a load across the first coil 222. For example, the current-sensing circuit 238 can include a first or current-sense resistor 240 to measure voltage across the first coil 222, which can aid in monitoring, controlling, and calibrating power transfer between the transmitter 202 and the receiver 204.

Further, the first coil interface 218 can include a full-bridge circuit 242 to power or drive the first coil 222. In some aspects, the full-bridge circuit 242 includes a plurality of field effect transistors (FETs) 244, which can be controlled by a first gate drive circuit 246 and a second gate drive circuit 248. Specifically, the gate drive circuits 246, 248 can be configured to receive pulse-width modulation output signals from the first controller 214 to operate the FETs 244 and selectively power the first coil 222. For example, a first FET 244A and a second FET 244B can be used to define a first half of the full-bridge circuit 242, while a third FET 244C and a fourth FET 244D can be used to define a second half of the full-bridge circuit 242. For full power operation, the halves of the full-bridge circuit 242 may operate out of phase. For example, the first and fourth FETs 244A, 244D can be powered on at the same time as one another, and the second and third FETs 244B, 244C can be powered on at the same time as one another. In this way, the phase shift between the two halves of the full-bridge circuit 242 can be varied, thereby allowing a voltage across the first coil 222 to be selected as desired. This can allow a generally constant voltage to be maintained within the receiver 204 as the distance between the transmitter 202 and receiver 204 varies (see FIG. 2), which in turn can increase the stability of data and/or power transmission from the transmitter 202.

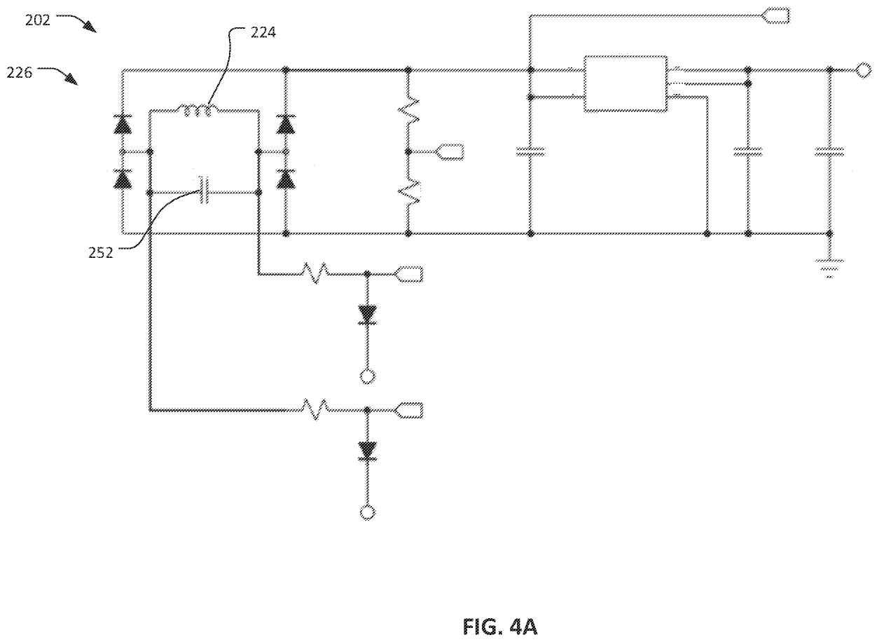

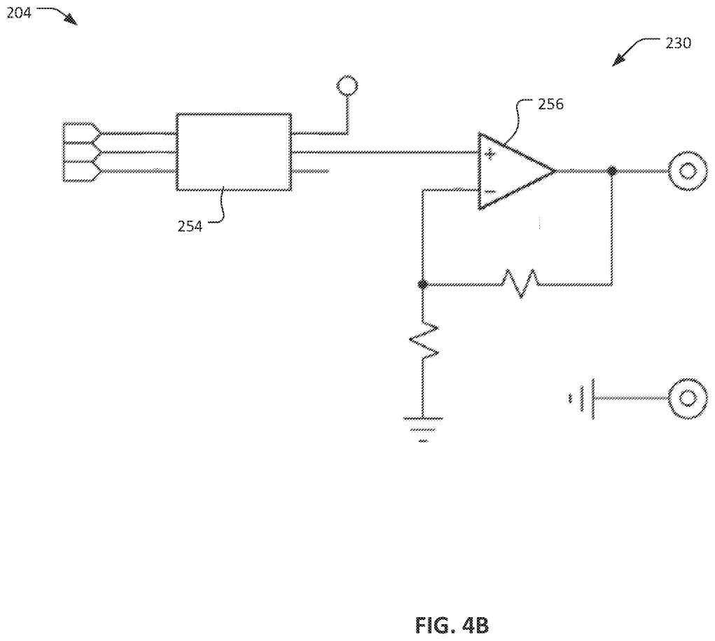

Referring now to FIGS. 4A and 4B, schematic diagrams are illustrated of the components of the receiver 204. As discussed above, the receiver 204 can receive instructions and/or power from the transmitter 202 (see FIG. 2) and generate a stimulation signal to output to a region of interest in a subject. Referring specifically to FIG. 4A, the second coil interface 226 of the receiver 204 can be implemented as an air-core transformer, similar to the first coil interface 218 of the transmitter 202 (see FIG. 3B). For example, the second coil 224 can include between 24 and 72 turns, or about 36 turns arranged as nine turns on each of four layers of the receiver 204. Further, the second coil 224 can be coupled to a second or resonant capacitor 252 to form a resonant circuit within the receiver 204. That is, the second capacitor 252 can be chosen to resonate with the second coil 224 at an operating frequency thereof to improve power transfer and operational efficiency.

Referring now to FIG. 4B, the stimulation signal generator 230 can be arranged as a monophasic generator, a biphasic generator, or another suitable type of generator. For example, the stimulation signal generator 230 may comprise a D/A converter 254 and an amplifier 256. In operation, the transmitter 202 can transmit instructions to the second controller 228, which in turn can operate the D/A converter 254 and the amplifier 256 to generate a stimulation signal. In some examples, the D/A converter 254 and the amplifier 256 define different aspects of the stimulation signal. For example, the transmitter 202 (see FIG. 2) can determine a frequency of the stimulation signal, the second controller 228 can determine a width of the stimulation signal, and the D/A converter 254 can determine an amplitude of the stimulation signal. Moreover, the output of the D/A converter 254, e.g., a stimulation pulse wave with a defined frequency, width, and amplitude, can be amplified using the amplifier 256. In some examples, the amplifier 256 is an operation amplifier programmed for a gain of between 1× and about 100×, or between 1× and about 10×, or about 2×. In this way, the stimulation signal generator 230 can generate a stimulation signal corresponding to the EMG signal recorded by the plurality of sensors 206 (see FIG. 2).

As discussed above, a computing device, e.g., the computing device 220 (see FIG. 2), can be used to execute one or more control processes to regulate the generation of the stimulation signal, and, more specifically, can set a first or activation voltage threshold and a second or termination voltage threshold that may correspond to intended VF function. In some aspects, EMG signals collected by the first controller 214 can be compared to these threshold voltages to determine if a stimulation signal should be generated, i.e., if a VF movement is desired. Specifically, a controller in a transmitter, e.g., the first controller 214 (see FIG. 2) can collect and analyze a plurality of EMG signal samples, e.g., between about 10 samples and about 500 samples, or between about 10 samples and about 100 samples, or about 50 samples. In particular, the controller can determine an average voltage, or an average difference of each sample from an average voltage, of the plurality of EMG signal samples, and compare the resulting value to the first and second voltage thresholds. If the resulting value is greater than the first threshold voltage, the controller can set the transmitter to a first power mode, i.e., a high power mode, and generate an activation signal, i.e., instructions, to send to a receiver, which in turn can cause the receiver to generate and output a stimulation signal. This output can be proportional to the average voltage and/or magnitude of EMG signal samples. However, if the resulting value is less than the second threshold voltage, the controller can set the transmitter to a second power mode, i.e., a low-power mode, and may not generate an activation signal, meaning that a stimulation signal also may not be generated. As discussed above, the first threshold can be greater than the second threshold, such that a threshold window exists between the first threshold and the second threshold. In some aspects, the threshold window defined by the dual thresholds, i.e., the first activation and second termination thresholds, prevents rapid on/off switching of the stimulation signal for EMG levels varying near these thresholds. That is, if the value returned after processing the EMG signals is between the first and second thresholds, the controller can selectively generate an activation signal based on the power mode of the transmitter to prevent rapid on/off switching. Specifically, when the resulting value is in between the first and second thresholds, the controller may generate the activation signal if the transmitter is in the first power mode, but may not generate the activation signal if the transmitter is in the second power mode. Thus, the LFES system utilizes a dual threshold system to prevent rapid on/off switching of the simulation signal, further improving stimulation signal generation efficacy.

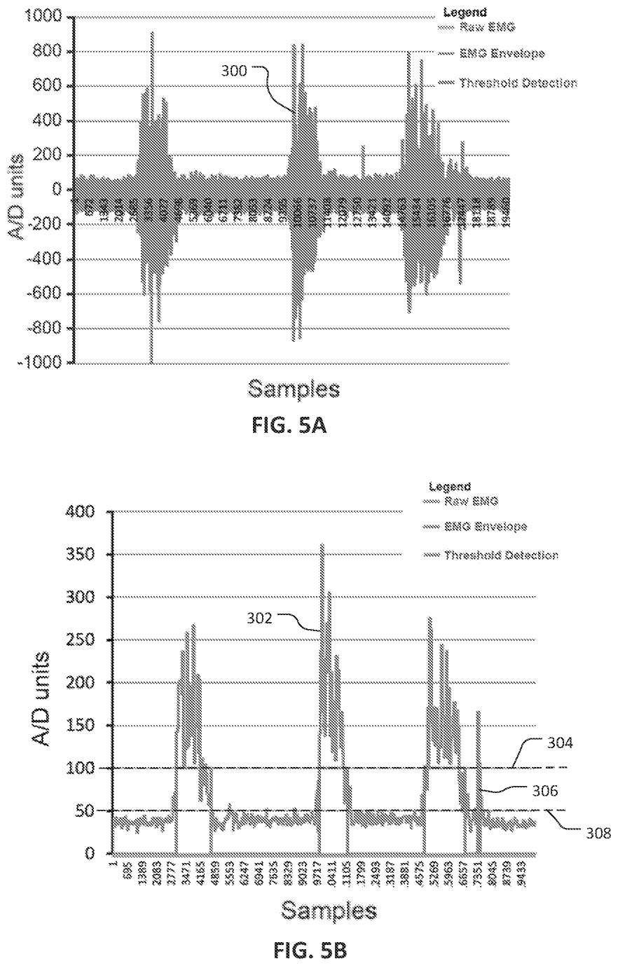

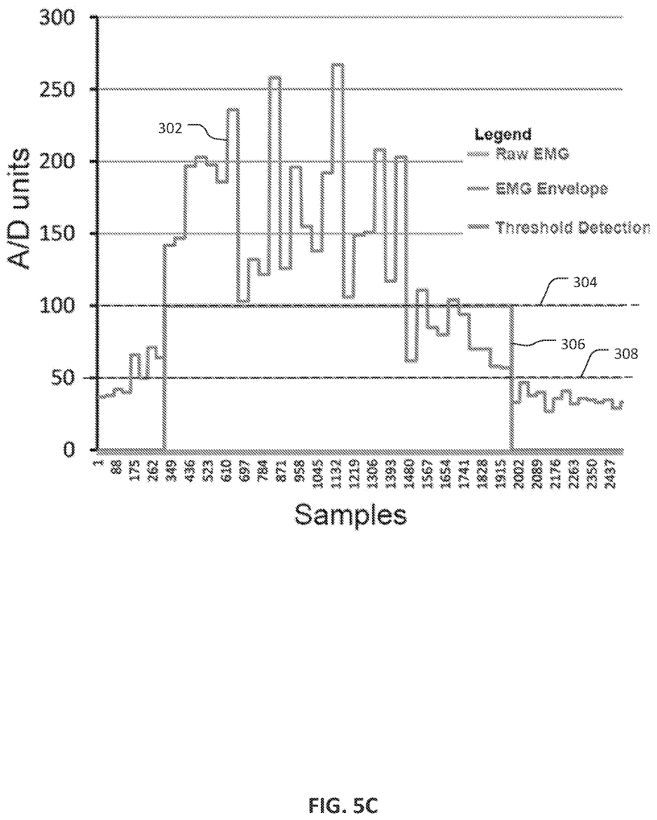

In this way, the LFES system can selectively stimulate a subject's impaired VF, or impaired VFs, based on a determination that VF function is intended, e.g., through analysis of EMG signals generated by the subject. That is, the LFES system utilizes a closed-loop feedback system to ensure that stimulation corresponds directly to the intended VF movement while preventing unstable stimulation, e.g., jittery or irregular stimulation. FIGS. 5A-5D are plots illustrating the processing of an EMG signal to provide stable stimulation. Specifically, FIG. 5A depicts a raw EMG signal 300 that is detected and recorded using sensors. As shown, the raw EMG signal 300 includes brief positive and negative signal spikes that makes comparison with threshold values difficult. To better represent the average EMG signal strength across time, the raw EMG signal 300 can be provided to a processor in the transmitter, e.g., the first processor 212 (see FIG. 2), to produce a processed EMG signal, or EMG envelope 302, as illustrated in FIGS. 3B and 3C. In particular, the EMG envelop 302 is the result of processing the raw EMG signal 300 using 50 sample time frames by determining the average voltage of the samples, the difference between each sample and the determined average voltage, and the absolute value of the determined difference. From this EMG envelop 302, appropriate threshold voltages can be determined, e.g., using the computing device 220 (see FIG. 2). For example, and as illustrated in FIGS. 5B and 5C, a first or activation threshold voltage can be set at line 304 falling at 100 A/D units on the plot's Y-axis, meaning that a stimulation signal is only generated when the magnitude of the EMG envelope 302 is greater than or equal to the first threshold voltage, as indicated by threshold detection signal 306, i.e., an activation signal. Further, a second or termination threshold voltage can be set at line 308, falling at 50 A/D units on the plot's Y-axis, meaning that the stimulation signal is only terminated when the magnitude of the EMG envelope 302 is less than or equal to the second threshold voltage. In the illustrated non-limiting example, the threshold detection signal 306 is terminated when the EMG envelope 302 falls below the second threshold voltage at line 308. By incorporating independent activation and termination thresholds that have different values than one another, the LFES provides hysteresis to reduce unnecessary triggering or stimulation, thereby improving the efficacy of the LFES system, along with subject safety and satisfaction.

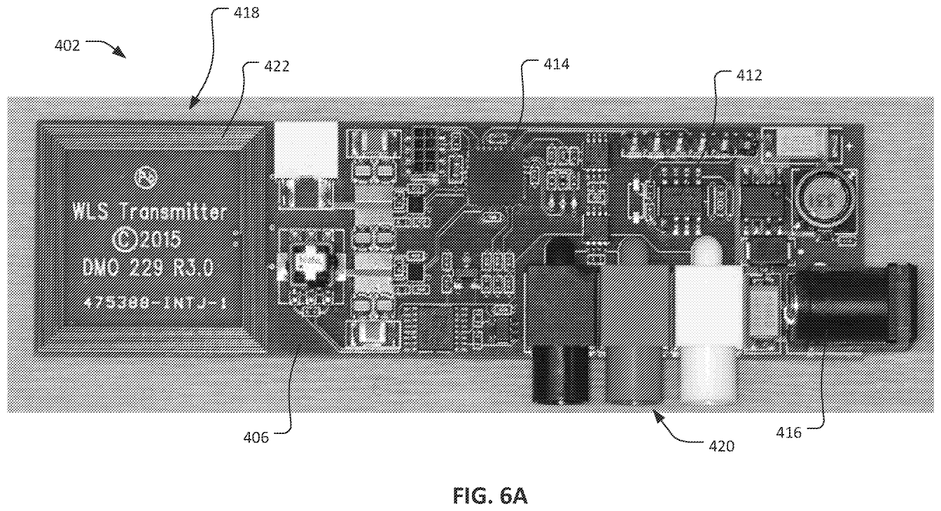

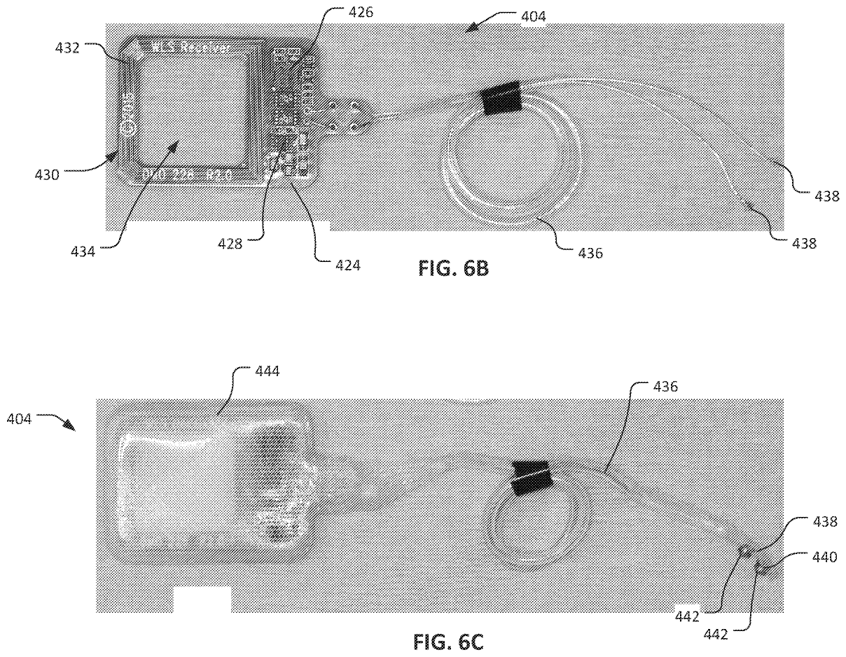

In some examples, a receiver and a transmitter of an LFES system can be arranged on separate printed circuit boards (PCBs). For example, a transmitter can be arranged on a first PCB that is located external to a subject during operation of the LFES system, while a receiver can be arranged on a second PCB that is configured to be surgically implanted within a subject, e.g., below the skin of a subject's neck or adjacent body location. Referring now to FIGS. 6A-6C, an example transmitter 402 and an example receiver 404 are illustrated which are similar to the transmitter 202 and receiver 204 discussed above with respect to FIG. 2. With particular reference to FIG. 6A, the transmitter 402 can include a first PCB 406 on which the various components of the transmitter 402 are disposed, including a first processor 412, a first controller 414, a computer interface 416, and a first coil interface 418, and a sensor interface 420. As discussed above, the first coil interface 418 can include a first coil 422, and the first coil 422 can have a plurality of turns, e.g., 46 turns. Further, the sensor interface 420 can be configured to couple the transmitter 402 to a plurality of sensors (not shown). It will be understood that the arrangement illustrated in FIG. 6A is an example of a transmitter 402 and that the components thereof can be arranged at various locations on the first PCB 406.

Referring now to FIG. 6B, the receiver 404 can include a second PCB 424 on which various components of the transmitter 402 can be disposed, including a second controller 426, a stimulation signal generator 428, and a second coil interface 430 that includes a second coil 432. As discussed above, the second coil 432 can have a plurality of turns, e.g., 46 turns. In some aspects, the second PCB 424 can define an aperture 434 around which the second coil 432 is wrapped. The aperture 434 can be included during manufacture of the second PCB 424 to decrease a weight thereof and/or increase flexibility thereof, which in turn can make implantation of the receiver 404 easier. In some aspects, a shape of the second coil 432 corresponds to a shape of the first coil 422 (see FIG. 6A), which may advantageously increase communication stability during operation of the transmitter 402 (see FIG. 6A) and the receiver 404.

Further, one or more connecting members 436, e.g., wires, can be coupled to the second PCB 424. More specifically, the connecting members 436 can be coupled to the stimulation signal generator 428 to output the stimulation signal from the receiver 404 at distal ends 438 thereof. Referring now to FIG. 6C, the receiver 404 is illustrated as including electrodes 440 coupled to the distal ends 438 of the connecting members 436. Thus, it will be understood that a stimulation signal, after having been generated by the stimulation signal generator 428, can be conducted through the connecting members 436 and output to an area of interest in a subject via the electrodes 440. In some aspects, the electrodes 440 define disk-like contacts 442 that are placed in contact with an impaired VF and/or extra-laryngeal muscles of a subject, as will be discussed below in greater detail. Further, the receiver 404, including the second PCB 424 and the connecting members 436, can be completely enclosed, i.e., encapsulated, by a protective sleeve or coating 444 to ensure the longevity of the receiver 404 and the safety of the subject after implantation of the receiver 404. It is contemplated that the protective coating 444 can comprise any suitable material, such as, e.g., epoxy, silicone, ceramic, titanium-based alloys, zirconium-based alloys, and/or ceramic compositions. In addition, it will be understood that the arrangements illustrated in FIGS. 6B and 6C are examples of a receiver 404 and that the components thereof can be arranged at various locations on the second PCB 424.

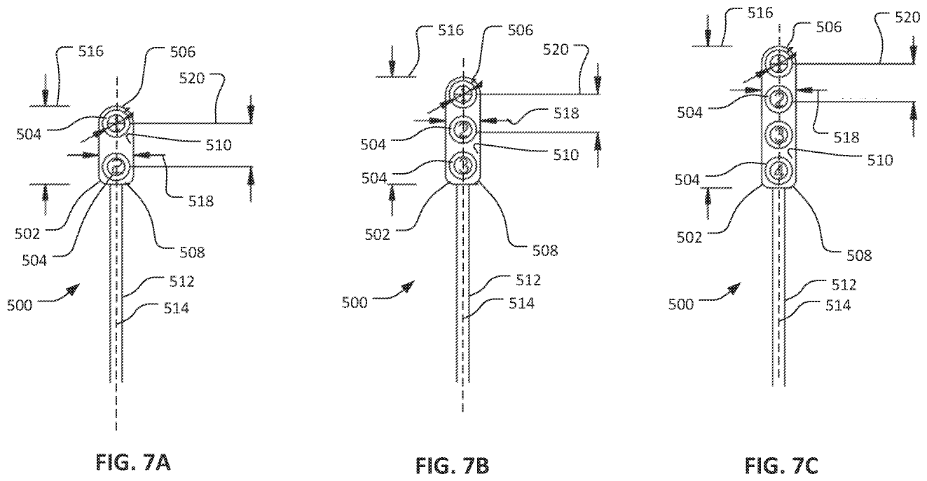

As discussed above, an LFES system can include a plurality of electrodes to provide a stimulation signal to an area of interest in a subject, e.g., a subject's impaired VF and/or extra-laryngeal muscles. It is contemplated that a variety of electrodes may be used to stimulate a subject's VF, including, for example, cylindrical electrodes, single-sided electrodes, double-sided electrodes, bipolar electrodes, tripolar electrodes, epimysial electrodes, intramuscular electrodes, nerve cuffs, etc. In the non-limiting examples illustrated in FIGS. 7A-7C, an electrode 500 can include a base 502 and a plurality of contacts 504 disposed on the base 502. While the contacts 504 are illustrated as disk-like contacts, it will be understood that the contacts 504 can be provided in any suitable shape. Furthermore, the contacts 504 can be provided in any suitable material for high electrical conductivity appropriate for implantation into living tissue, including, for example, platinum, platinum-iridium, gold, stainless steel, conductive polymers, etc. Moreover, the base 502 can define a first or distal end 506, a second or proximal end 508 opposite the first end, a first side 510, and a second side (not shown) opposite the first side 510. In some aspects, the first end 506 of the base 502 is rounded, while the second end 508 of the base 502 is substantially flat. In some aspects, a connecting member 512, e.g., a stainless steel wire, is coupled to the second end 508 of the base 502 and provides the electrode 500 with a simulation signal, as discussed above. In some examples, the connecting member 512 includes a plurality of independent wires that correspond to each contact of the plurality of contacts 504.

Further, the plurality of contacts 504 can be disposed on the first side 510 and/or the second side (not shown) of the base 502. It is contemplated that the plurality of contacts 504 can be arranged in any suitable configuration on the base 502. For example, the plurality of contacts 504 can be axially aligned along a longitudinal axis 514 that extends through the first end 506 and the second end 508 of the base 502. It is contemplated that any number of contacts may be disposed on the base 502, such as two, three, or four contacts 504, as shown in FIGS. 7A-7C, respectively. In other examples, an electrode can include one, five, six, ten, and/or more than ten contacts arranged in a single row, as shown in FIGS. 7A-7C, or arranged in multiple rows or patterns. The base 502 can define a height 516 that is measured between the first end 506 and the second end 508 thereof in a direction that is parallel with respect to the longitudinal axis 514, and a width 518 that is measured in a direction that is perpendicular with respect to the longitudinal axis 514. Further, each of the contacts 504 can be spaced by one another by a distance 520 that is measured in a direction that is parallel with respect to the longitudinal axis 514. In some examples, the width 518 of the base 502 is between about 10% and about 60% of the height 516 of the base 502, or between about 25% and about 55% of the height 516 of the base 502. In some examples, the distance 520 is between about 10% and about 60% of the height 516 of the base 502, or between about 25% and about 55% of the height 516 of the base 502. In some aspects, an electrode can include more than one base to provide flexibility during implantation, as will be discussed below in greater detail.

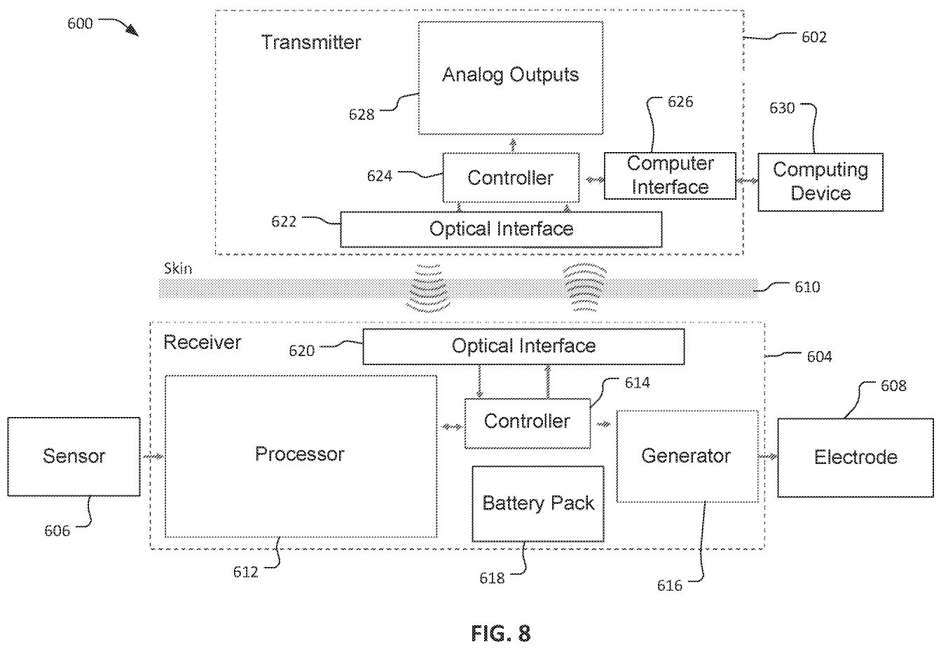

Referring now to the non-limiting example illustrated in FIG. 8, a closed-loop LFES system (e.g., a battery-powered LFES system 600) can be used to restore and/or enhance a subject's VF function by detecting a subject's biosignals associated with normal VF function and stimulate an impaired function with a stimulation signal. It will be understood that any components of the LFES system 600 discussed herein may be implemented in the LFES system 200 as discussed for FIG. 2, and vice versa. Thus, the LFES systems 200, 600 may be similar and/or compatible with one another, meaning that the LFES system 600 may include a transmitter, a receiver that is controlled by the transmitter, a plurality of sensors to detect an EMG signal, and a plurality of electrodes to output a stimulation signal that is generated by the receiver. However, the LFES system 600 may differ from the LFES system 200 in some aspects. Specifically, the LFES system 600 may be arranged such that the plurality of sensors are coupled to the receiver 604 rather than the second controller 624, and the receiver 604 may be capable of processing EMG signals and generating a stimulation signal independent of the external transmitter 602. Further, the LFES system 600 can include a wireless communication interface, e.g., an optical interface rather than a coil interface, and the LFES system 600 may be at least partially powered by a battery. Furthermore, the LFES system 600 may include electrically inductive elements, e.g., those of the LFES system 200 (see FIG. 2), to power the receiver 604 and to charge the battery pack 618. Accordingly, the LFES system 600 is another example of an LFES system, according to the present disclosure.

As illustrated in FIG. 8, an LFES system 600 can include a transmitter 602, a receiver 604, a plurality of sensors 606, and a plurality of electrodes 608. The transmitter 602 and the receiver 604 can be provided within a single device, or the transmitter 602 and the receiver 604 can be provided as separate devices that are electrically coupled to one another. In some aspects, the receiver 604 can be configured to be implanted within a subject, e.g., within a subject's neck, a head, a thorax, an abdomen, and/or an adjacent body location for the electrode 608 to reach the subject's VF(s), whereas the transmitter 602 can be configured as an external device. That is, the receiver 604 can be surgically implanted under a subject's skin 610, while the transmitter 602 can be arranged external to the subject's skin 610. The transmitter 602 can be configured to monitor the EMG signals processed by the receiver 604 and initially program the receiver 604, e.g., program threshold levels, stimulation signal parameters, EMG processing parameters, etc. Correspondingly, the receiver 604 can be configured to generate and provide the stimulation signal to a region of interest in the subject via the plurality of electrodes 608 after receiving and processing EMG signals provided by the sensors 606, as will be discussed below in greater detail.

Specifically, the receiver 604 can be coupled to the plurality of sensors 606, and the plurality of sensors 606 can also be surgically implanted within a subject's larynx and/or VF(s), as will be discussed below in greater detail. It is contemplated that any suitable sensor may be used for the plurality of sensors 606, such as, e.g., EMG recording electrodes, magnetic field sensors, pressure sensors, strain or bend gauge sensors, accelerometers, bioimpedance sensors, gyroscopes, displacement sensors, microphones, central and peripheral nervous system recordings, etc. Accordingly, the plurality of sensors 606 can be configured to detect the intended movement of the subject's larynx and/or intended VF function. The receiver 604 can receive the EMG signals or other signals recorded by the plurality of sensors 606 and transmit the EMG signals or other signals to the transmitter 602 for processing, or the receiver 604 can include internal circuitry to process the EMG signals or other signals.

To that end, the receiver 604 can include a variety of components, such as a first processor 612, a first controller 614, a stimulation signal generator 616, a battery pack 618, and a first optical interface 620. In some aspects, the first processor 612 includes an instrumentation amplifier that processes the EMG signal recorded by the plurality of sensors 606. For example, the first processor 612 can include a variety of filtering components to remove any common-mode noise from the EMG signal and amplify the resulting signal by a gain, as discussed above. In some aspects, the first processor 612 can also be used with the first controller 614 to generate a stimulation signal. Specifically, the first controller 614 can be configured to control the operation of the receiver 604, including processing the EMG signal, interfacing with the transmitter 602, operating the first processor 612 and/or the stimulation signal generator 616 to generate a stimulation signal, and/or providing a software blanking command to specify a number of EMG samples that should be ignored each time a stimulation pulse is generated. In some aspects, the stimulation signal generator 616 includes at least one amplifier and/or at least one digital-to-analog (D/A) converter, as will be discussed below in greater detail. The first controller 614 may also be configured to control power consumption of the receiver 604, i.e., power drawn from the battery pack 618. Correspondingly, coupling the battery pack 618 to the receiver 604 allows the receiver to function autonomously, that is, without requiring power from the transmitter 602.

With continued reference to FIG. 8, communication between the transmitter 602 and the receiver 604 is provided via a wireless communication interface, e.g., an inductive interface, an optical interface, a radiofrequency (RF) interface, or any combination thereof. For example, a wireless communication interface can comprise the first optical interface 620 of the receiver 604 and a second optical interface 622 of the receiver 604. To communicate data between the transmitter 602 and the receiver 604, infrared light can be transmitted across between the optical interfaces 620, 622 using light-emitting diodes (LEDs) and photodetectors, as will be discussed below in greater detail. After the raw EMG signal obtained by the plurality of sensors 606 has been obtained and processed using the first processor 612, the receiver 604 can provide the processed EMG signal to the transmitter 602 via the optical interfaces 620, 622. The transmitter 602 can be used to verify the proper function of the receiver 604 and to adjust or select first processor 612 parameters, such as gain, activation/termination thresholds, blanking period, and/or stimulation level to optimize functional electrical stimulation. These adjustments could be made by an operator or by using encoded algorithms. After the first processor 612 has been adjusted with the transmitter 602, the receiver 604 can function autonomously. However, the transmitter 602 may be used to monitor EMG signals provided to the receiver 604, as discussed below.

Correspondingly, the transmitter 602 can include a variety of components, including the second optical interface 622, a second controller 624, a computer interface 626, and one or more analog outputs 628. The second controller 624 can be configured to control operation of the transmitter 602, including interfacing with the receiver 604 and/or an external computing device 630, and monitoring an EMG signal with the one or more analog outputs 628. The computer interface 626 of the transmitter 602 can be configured as a UART interface, a Bluetooth interface, a USB interface, or another type of interface. In some aspects, the computing device 630 can be used to automatically or manually, e.g., by a human operator, monitor and control operation of the LFES system 600. More specifically, the computing device 630 can be used to adjust or select parameters of the first processor 612 of the receiver 604, and the receiver 604 can function autonomously after the parameters have been adjusted. Moreover, in some aspects, the computing device 630 is configured as a monitoring system, and the computing device 630 can be configured to execute a variety of control processes to regulate stimulation signal generation, including setting threshold voltages, as discussed above. In some aspects, the analog outputs 628 are configured to allow an operator to monitor key analog signals associated with the LFES system 600 for data acquisition and display, including the raw EMG signal, the processed EMG signal, i.e., EMG envelope, an activation signal, and/or blanking intervals, as will be discussed below in greater detail. For example, the analog outputs 628 can be configured to couple the transmitter 602 to a monitoring device (not shown), e.g., an oscilloscope.

Thus, in operation, the LFES system 600 can record EMG signals with the plurality of sensors 606, and the raw EMG signal can be processed via the first processor 612, drawing power from the battery pack 618. In some aspects, parameters of the first processor 612 are adjusted with the transmitter 602 to allow the receiver 604 to function autonomously. The first controller 614 can then provide the processed EMG signal to the transmitter 602 via the first and second optical interfaces 620, 622 of the receiver 604 and the transmitter 602, respectively. The second controller 624 can provide the processed EMG signal to the analog outputs 628, which allow an operator to monitor key signals of the LFES system 600 for subject safety. In some aspects, the computing device 630 is also configured to monitor the key signals of the LFES system 600 by executing, for example, one or more control processes. In parallel, the first controller 614 may further process the EMG signal and/or determine that a stimulation signal should be generated based on the voltage of the processed EMG signal. If the first controller 614 determines that a stimulation signal should be generated, the first controller 614 can transmit an activation signal, i.e., encoded instructions, to the stimulation signal generator 616. Put another way, the first controller 614 can operate the stimulation signal generator 616 to generate and output the desired stimulation signal to a region of interest in the subject.

In some aspects, the first and second optical interfaces 620, 622 utilize LEDs and photodetectors to allow communication between the transmitter 602 and the receiver 604. Referring now to FIG. 9, the first optical interface 620 can be a serial interface that includes a first infrared LED 632 to transmit optical, i.e., serial, data and a first phototransistor 634 to receive optical data. In some aspects, the first phototransistor 634 can be coupled to a current mirror 636 that measures the current in a single transistor and creates a current in a second transistor that is equal to the measured current. Further, to minimize power consumption in the receiver 604, current in the first LED 632 can be held to a minimum. During data communication over the first optical interface 620, two bytes of data for each sample of EMG data can be sent to the transmitter 602. For example, the first byte of data can contain six bits of EMG amplitude data and two bits of identification information. The second byte can contain four bits of EMG amplitude data, two bits of flag data, and two bits of identification information. One of the bits of flag data can indicate if a valid EMG signal has been detected, and the other bit of flag data can indicate if the EMG signal is currently being blanked or ignored. In this way, the raw data and the bits of flag data can allow the transmitter 602 (see FIG. 8) to duplicate the determinations performed by the receiver 604 and display diagnostic data to the operator as a result. Thus, the receiver 604 does not have to transfer the determinations over the optical interfaces 620, 622, which increase the battery life of the battery pack 618, and, by extension, the receiver 604.

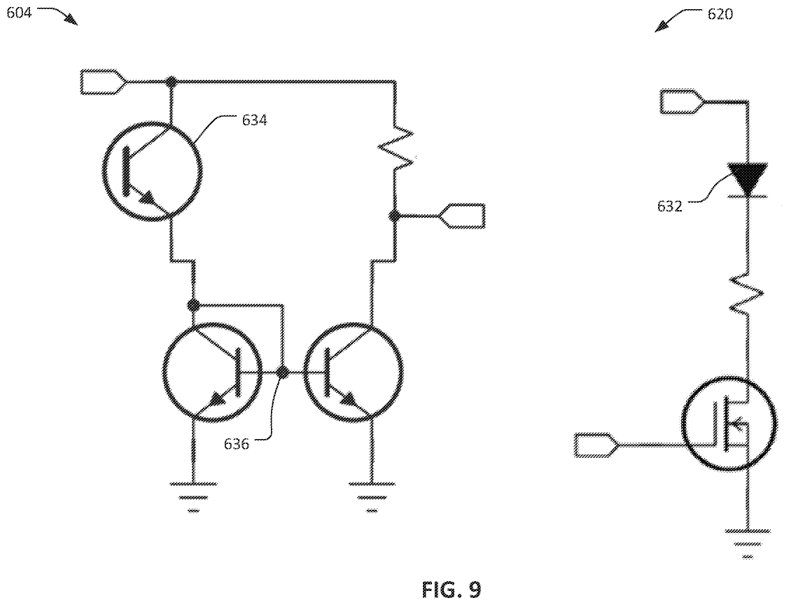

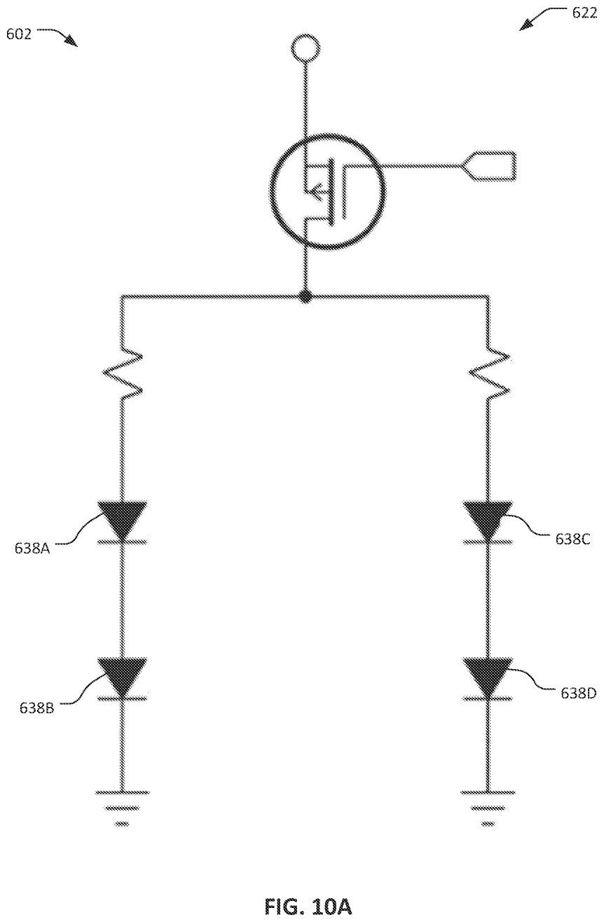

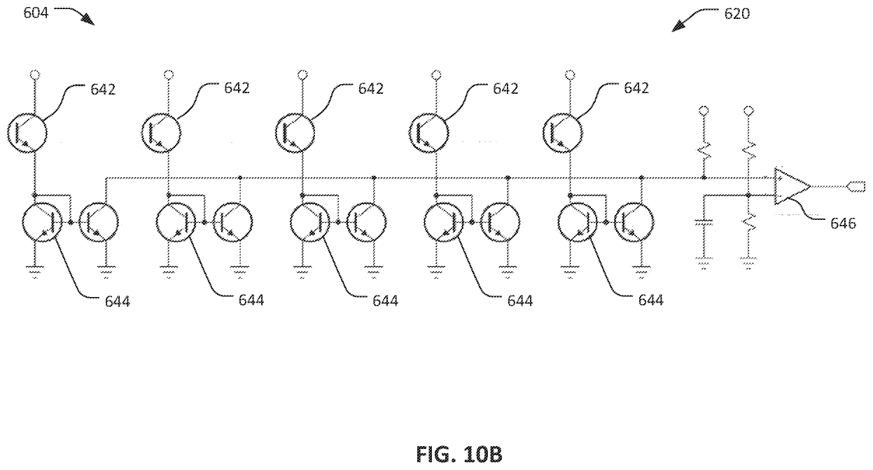

Referring now to FIGS. 10A and 10B, the second optical interface 622 can also be a serial interface having LEDs and phototransistors to transmit and receive data. With specific reference to FIG. 10B, the second optical interface 622 can include a plurality of LEDs 638 to transmit optical, i.e., serial, data. In some aspects, the LEDs 638 are arranged in a circle on the transmitter 602 and are driven in parallel with a current that is sufficient to penetrate a subject's skin that separates the transmitter 602 and the receiver 604. Further, the transmitter 602 may draw power from the computing device 630 rather than the battery pack 618 to perform this function, so as to further increase battery life of the receiver 604 (see FIG. 8). In some aspects, the LEDs 638 are also configured to provide status indications to an operator. For example, a first LED 638A can be configured to emit a first signal, e.g., a green light, when the transmitter 602 is wireless coupled or connected to the receiver 604 (see FIG. 8), a second LED 638B can be configured to emit a second signal, e.g., a red light, when the receiver 604 (see FIG. 8) detects an EMG signal that falls within a threshold voltage window, and a third LED 638C can be configured to emit a third signal, e.g., a blue light, when blanking occurs. In other examples, the LEDs 638 are configured to emit additional status indicators, or the transmitter 602 can include additional LEDs that are separate from the plurality of LEDs 638 to provide status indicators.

Referring now to FIG. 10B, the second optical interface 622 can also include a plurality of phototransistors 642 to receive optical data, and the plurality of phototransistors 642 can be arranged in a circle, i.e., in parallel, on the transmitter 602. Each phototransistor 642 can be coupled to a corresponding current mirror 644, and all of the current mirrors can also be coupled in parallel to allow signals from the phototransistors 642 to be summed together. The resulting summed signal can be provided to a comparator 646 to demodulate the summed signal before being provided to the second controller 228 (see FIG. 8).

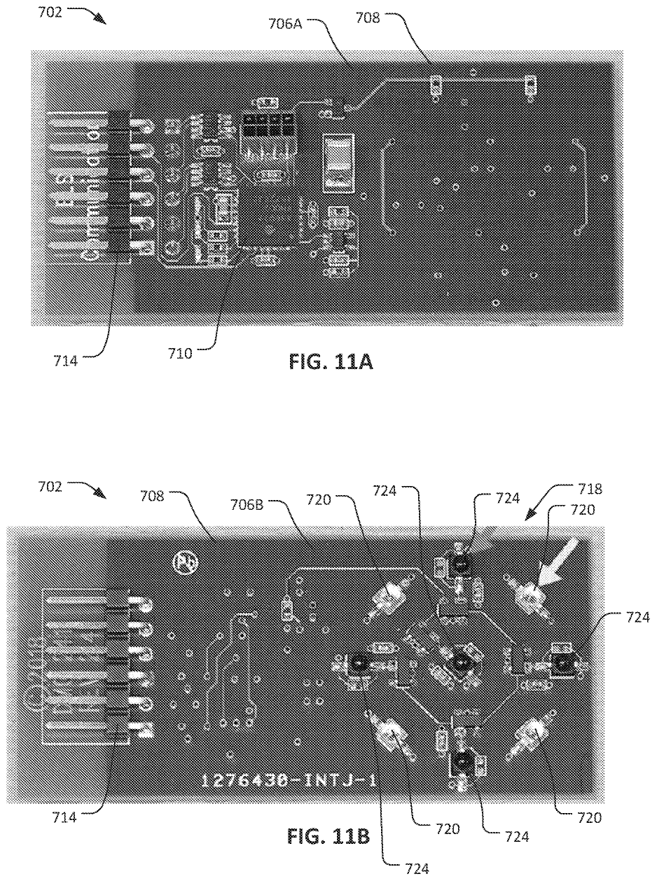

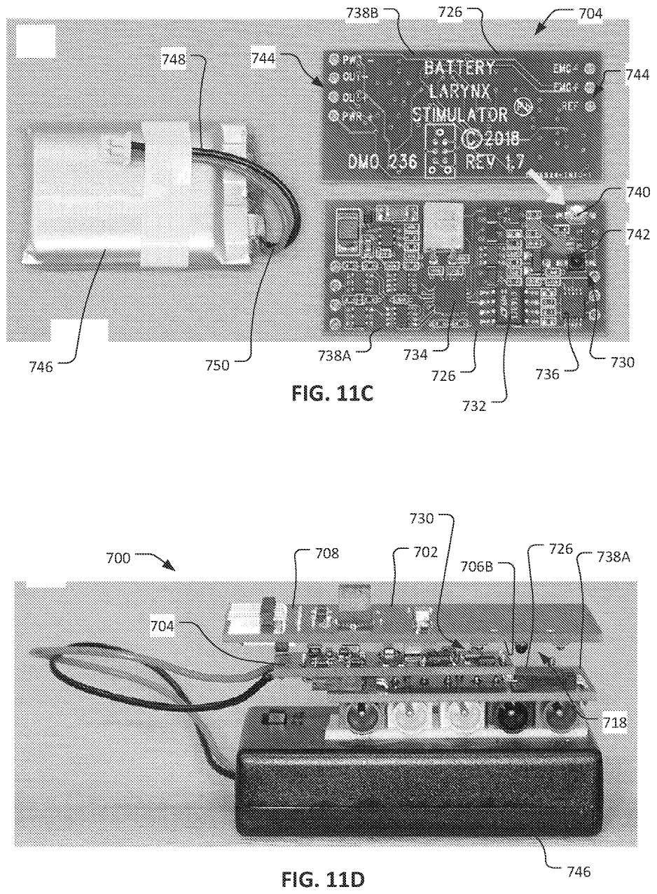

As discussed above, a receiver and a transmitter of an LFES system can be arranged on printed circuit boards (PCBs). Further, a receiver and a transmitter can be aligned with one another during operation such that a first optical interface of the transmitter is within a predetermined distance from a second optical interface of the receiver, the optical interfaces being arranged on separate PCBs. Referring now to FIGS. 11A-11D, an example transmitter 702 and an example receiver 704 are illustrated which are similar to the transmitter 602 and receiver 604 discussed above with respect to FIG. 8.

With particular reference to FIG. 11A, a top or first side 706A of a first PCB 708 of the transmitter 702 is illustrated. Various components of the transmitter 702 can be disposed on the first side 706A of the first PCB 708, including, for example, a first controller 710 and a plurality of connectors 714. In some aspects, the plurality of connectors 714 are pin connectors that comprise a computer interface and a plurality of analog outputs, as discussed above. Referring now to FIG. 11B, a bottom or second side 706B of the first PCB 708 is illustrated, including a first optical interface 718. Similar to the second optical interface 622 discussed above (see FIGS. 10A and 10B), the first optical interface 718 can include a plurality of LEDs 720 and a plurality of phototransistors 724. In some aspects, the LEDs 720 and the phototransistors 724 can be arranged in a particular configuration, shape, or pattern to improve wireless communication with the receiver 704 (see FIG. 11C). For example, the LEDs 720 and the phototransistors 724 can be arranged in a circle on the second side 706B of the first PCB 708, with one phototransistor 724 disposed within the center of the circle.

Referring now to FIG. 11C, the receiver 704 can include a second PCB 726 on which various components of the transmitter 402 can be disposed. For example, a second optical interface 730, a first processor 732, a second controller 734, and a stimulation signal generator 736 can be disposed on a top or first side 738A of the second PCB 726. Similar to the first optical interface 620 discussed above (see FIG. 9), the second optical interface 730 includes a first LED 740 and a first phototransistor 742 to transmit and receive optical data, respectively. Further, one or more ports 744 can be located in bottom or second side 738B of the second PCB 726, and the ports 744 can serve as connection points between various inputs and outputs of the LFES system. For example, the ports 744 can serve as power terminals, sensor terminals to receive EMG signals, ground terminals, and/or electrode terminals to output stimulation signals. Specifically, the receiver 704 can be powered by a battery pack 746 by coupling positive and negative connectors 748, 750 thereof to the ports 744. In some aspects, the battery pack 746 includes a high-capacity battery, a rechargeable battery, a lithium battery, multiple alkaline batteries, or any combination thereof. Thus, in some aspects, the battery pack 746 can be rechargeable without having to be removed from a subject's body, which allows the battery pack 746 to be chronically implanted within a subject.

Referring now to FIG. 11D, an LFES system 700 is illustrated as being assembled, meaning that the battery pack 746 is coupled to the receiver 704, and the transmitter 702 and the receiver 704 are arranged in an opposing configuration such that the optical interfaces 718, 730 are aligned with one another. Put another way, the transmitter 702 and the receiver 704 are arranged such that the second side 706B of the first PCB 708 faces the first side 738A of the second PCB 726. In practice, it will be understood that the transmitter 702 may be located outside of a subject's body while the receiver 704 and the battery pack 746 may be implanted within the subject's body, e.g., within a subject's neck or adjacent body location. In some aspects, communication of data between the transmitter 702 and the receiver 704 can be optimized by positioning the transmitter 702 within a predetermined distance with respect to the receiver 704. In some aspects, the predetermined distance is less than about 10.0 centimeters (cm), or less than about 8.0 cm, or less than about 5.0 cm, or less than about 3.0 cm, or less than about 1.0 cm.

As discussed above, an LFES system can utilize one or more controllers to execute control processes to regulate stimulation signal generation and/or prevent stimulation signals from corrupting raw EMG signals. For example, a controller, e.g., the first controller 614 (see FIG. 8) can be configured to control EMG signal sampling with a plurality of sensors, determine if the detected EMG signals fall within a threshold voltage window, and operate a stimulation signal generator, e.g., the stimulation signal generator 616 (see FIG. 8), upon such a determination. Further, a controller can be configured to prevent any stimulation signals that are generated from interfering with, e.g., corrupting, the raw EMG signals. For example, a controller can execute instructions to implement two rolling-average filters via circular buffers. The first rolling-average filter can be used to negate low-frequency drift in raw EMG signals by determining an average value of all EMG samples in a buffer, subtracting the average from a new sample, and determining the absolute value of the resulting difference. The output from this determination can be used as the value of a new sample, and a second rolling average of the magnitude of the new sample can also be performed to determine the average magnitude of the EMG signal. Further, the controller can compare the average magnitude of the EMG signal against predetermined and/or adjustable threshold levels to determine if a stimulation signal should be generated.

To accomplish this, a controller can disconnect raw EMG signals from a processor, e.g., the first processor 612 (see FIG. 8), and prevent rolling-average filters from being advanced during stimulation signal generation. EMG samples can be continuously taken during stimulation signal generation, but such samples may only be used for diagnostic purposes and may not be included in the threshold determination discussed above. Further, a controller can provide a software blanking command to specify a number of EMG samples that should be ignored each time a stimulation pulse is generated, further improving signal stability and stimulation signal generation accuracy.

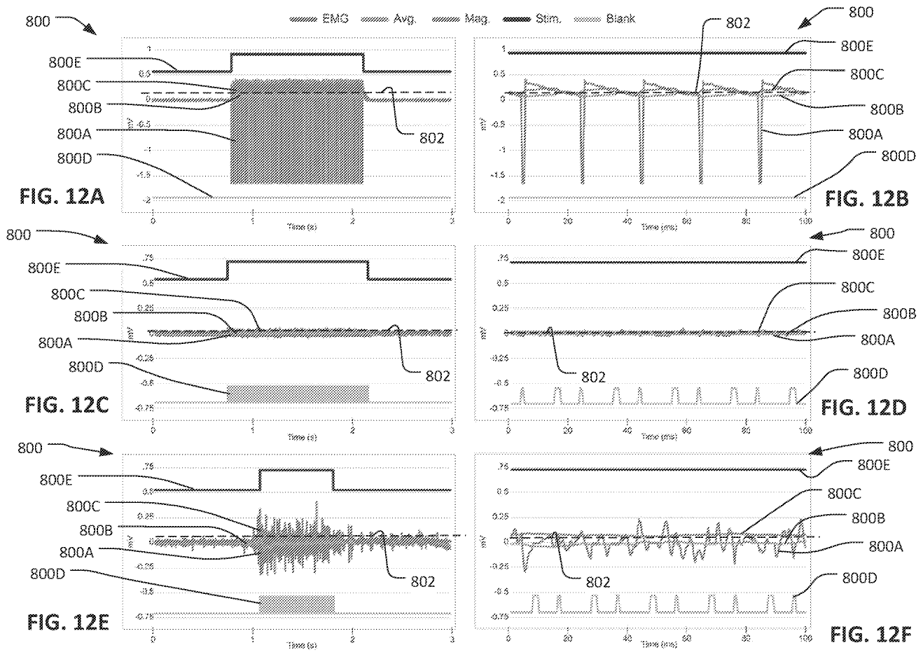

Referring now to FIGS. 12A-12F, a series of plots are illustrated which depict the effect of providing a software blanking command to reduce and/or eliminate shock artifacts to preserve raw EMG signal integrity. Specifically, FIGS. 12A-12F illustrate a series of signals 800, e.g., a raw EMG signal 800A, an average EMG voltage signal 800B, a magnitude EMG signal 800C, a stimulation signal 800D, and a blanking signal 800E measured as voltage over time. FIGS. 12A, 12C, and 12E depict the signals 800 measured over a first time window, e.g., zero seconds to three seconds, while FIGS. 12B, 12D, and 12F depict the signals 800 measured over a second time window that is shorter than the first time window, e.g., zero milliseconds (ms) to 100 ms.

Referring specifically to FIGS. 12A and 12B, the raw EMG signal 800A corresponds to a quiescent or baseline surface EMG measured from a subject's skin. The raw EMG signal 800A includes shock artifacts that may cause an amplifier in an LFES system to saturate, which in turn can lead to prolonged recovery and less accurate stimulation signal generation or self-perpetuating positive feedback, thereby causing continuous stimulation regardless of whether physiological EMG signal is present. To reduce these shock artifacts, the blanking signal 800E can be applied, as illustrated in FIGS. 12C and 12D. As a result, the raw EMG signal 800A can be more stable, thus leading to more stable average and magnitude EMG signals 800B, 800C. That is, the magnitude EMG signal 800C can be more easily compared to a threshold voltage level to determine if the stimulation signal 800D should be generated, as indicated by the step-function profile of the stimulation signal 800D in FIG. 12C. This in turn can lead to more accurate stimulation signal generation, meaning that stimulation signals can be more accurately mapped to intended function of a subject's VF(s).

FIGS. 12E and 12F depict the raw EMG signal 800A as a subject's muscles, e.g. a subject's VF and/or extra-laryngeal muscles, are actively contracted, thereby causing the raw, average, and magnitude EMG signals 800A, 800B, 800C to exceed a voltage threshold 802, i.e., an activation threshold. The blanking signal 800E can be provided to eliminate and/or reduce shock artifacts from the system, thus allowing a more stable magnitude EMG signal 800C to be determined, e.g., using the control processes discussed above, and compared to the voltage threshold 802. When the magnitude EMG signal 800C is determined to exceed the voltage threshold 802, the stimulation signal 800D can be generated and/or increased. In this way, using a blanking signal to reduce shock artifacts in raw EMG signals can lead to more efficient power consumption and more accurate VF stimulation.

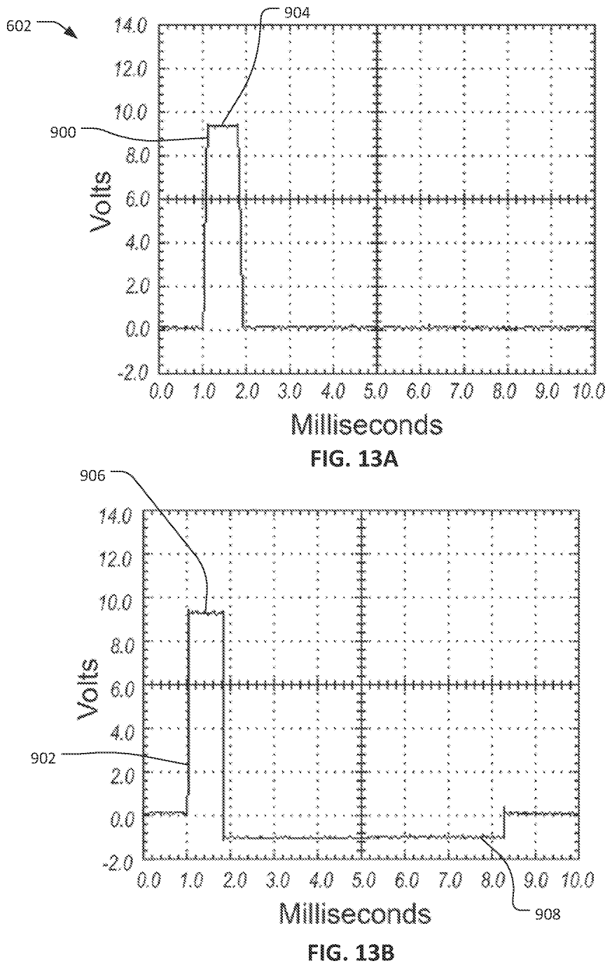

Correspondingly, an LFES system can generate a variety of stimulation signal profiles using a stimulation signal generator. For example, FIG. 13A depicts a first stimulation signal 900 that may be generated by a monophasic stimulation generator, e.g., the stimulation signal generator 230 (see FIG. 2), and FIG. 13B depicts a second stimulation signal 902 that may be generated by a biphasic stimulation generator, e.g., the stimulation signal generator 616 (see FIG. 8). Specifically, the first stimulation signal 900 may be a monophasic signal that includes a single stimulation phase 904, meaning that the stimulation signal returns to baseline, i.e., zero volts, after a single positive stimulation signal is generated. In contrast, the second stimulation signal 902 can be a biphasic signal that includes a positive first stimulation phase 906 and a negative second stimulation phase 908 before returning to baseline, i.e., zero volts. In some aspects, a magnitude of the first stimulation phase 906 is greater than the second stimulation phase 908, and a duration of the second stimulation phase 908 is greater than a duration of the first stimulation phase 906. Thus, the second stimulation signal 902 can be a charged-balanced biphasic pulse with an asymmetric waveform, which can further reduce possible artifacts in a detected EMG signal and minimize the toxic effects of charge imbalance at the interface between electrode 208 608 and the stimulated muscle.

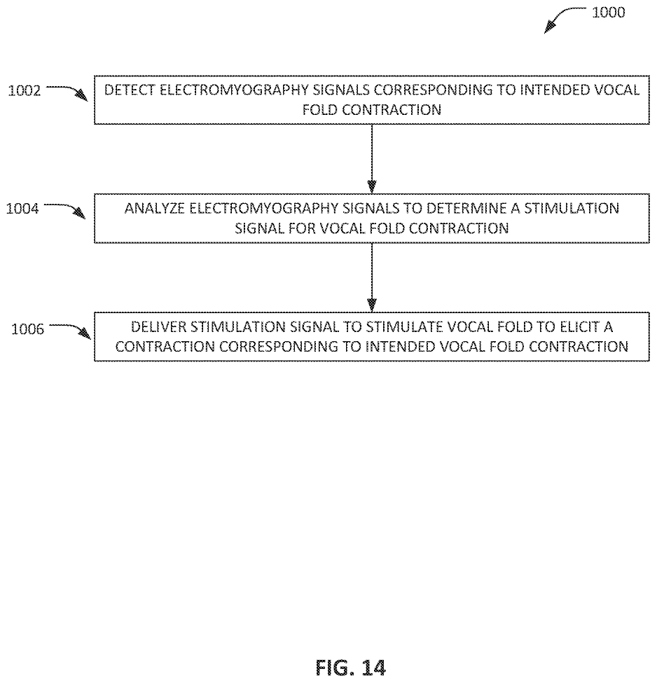

FIG. 14 illustrates a method of stimulating one or both of a subject's VF by determining an appropriate stimulation signal based on the subject's own EMG signals, generating such a stimulation signal, and providing the stimulation signal to an area of interest of a subject, e.g., the subject's impaired VF, according to some aspects of the present disclosure. As discussed above, using a closed-loop stimulation system, i.e., a system in which stimulation signals are generated based on a subject's own biosignals, allows artificial stimulation signals to more accurately reflect intended VF function, which in turn can increase overall efficiency of the system and lead to greater subject safety and satisfaction. In some aspects, intended VF function can be determined by analy zing a subject's EMG signals that correspond to neck muscle movement, such as EMG signals that are measured from a subject's functional VF or extra-laryngeal muscles. For example, a process 1000 of generating a stimulation signal with a closed-loop system can include first detecting EMG signals corresponding to intended contraction of one or both VFs of a subject at step 1002. As discussed above, detecting raw EMG signals can be performed with at least one sensors, e.g., sensing electrodes, that can be implanted within a subject's neck and coupled to an LFES system. For example, a plurality of sensors may be in contact with a subject's functional VF, if available, and/or a subject's extra-laryngeal muscles. At step 1004, the process 1000 can include analyzing the detected EMG signals to determine a stimulation signal for contracting the subject's VF(s). In some aspects, analyzing the EMG signals and determining a stimulation signal can include a variety of sub-steps to remove noise and artifacts from the raw EMG signals, and to execute one or more control processes to confirm a stimulation signal is appropriate, as will be described in greater detail below.

Still referring to FIG. 14, the process 1000 can further include sending an activation signal from a transmitter to a receiver, the activation signal including instructions for generating the stimulation signal. As discussed above, an LFES system can include one or more controllers to communicate an activation signal from an external device, i.e., a transmitter, to an implantable device, i.e., a receiver. In some aspects, the instructions for generating the stimulation signal are encoded in the form of serial data that is communicated across inductive or optical interfaces within the external device and the implantable device, and the instructions may include desired characteristics of a stimulation signal waveform, e.g., amplitude, frequency, pulse width, etc. The process 1000 can further include generating the stimulation signal, which may be performed by a stimulation signal generator included within the implantable device. As discussed above, a controller can be used to execute the instructions included in the activation signal and operate a stimulation signal generator accordingly.

At step 1006, the process 1000 can include delivering the stimulation signal to an area of interest of the subject to elicit a contraction of the VF(s) corresponding to intended contraction of the VF(s). In some aspects, intended contraction of a VF is determined by analyzing the EMG signals detected by the at least one sensor, as discussed above. Further, the stimulation signal can be delivered to an area of interest of the subject using an electrode that is coupled to the implantable device at step 1006. That is, the generated stimulation signal can be conducted along the electrodes and output to an area of interest of the subject, e.g., a subject's impaired VF(s).

In some aspects, step 1004, e.g., analyzing EMG signals to determine a stimulation signal for contracting a VF, can include processing a raw EMG signal to obtain a normalized or amplitude signal and then comparing the amplitude signal to a threshold voltage window. In particular, the amplitude signal can be compared to an activation threshold value and a termination threshold value. If the amplitude signal is greater than the termination threshold value, but does not reach or exceed the activation threshold value, no activation signal is generated. If the amplitude signal equals or exceeds the activation threshold value, an activation signal can be generated, and stimulation can continue for as long as the amplitude signal remains greater than the termination threshold value. The output can be proportional to the average voltage and/or magnitude of EMG signal samples in a linear or non-linear manner. The stimulation signal can be terminated if the amplitude signal is less than the termination threshold value. In some aspects, analysis and determination, i.e., generation, of a stimulation system can be performed by a processor of an LFES system. Accordingly, the LFES system can ensure that stimulation signal generation corresponds to intended VF function and mitigate if needed. Therefore, the LFES system disclosed herein can accurately generate and provide stimulation signals to a subject based on the subject's own biosignals. In this way, an LFES system can provide a closed-loop feedback approach for detecting a subject's intended VF function and stimulating a subject's impaired VF(s).

In other examples, other processes can be used to determine and deliver a stimulation signal to elicit contraction of a subject's VF(s). For example, a process for laryngeal stimulation can include securing at least one sensor to a subject and recording EMG signals of a subject with the at least one sensor. The process can further include receiving the recorded EMG signals and generating a stimulation signal that is configured to stimulate one or both VFs of a subject to perform at least of treating glottic insufficiency or improving airway protection. For example, a processor can analyze the recorded EMG signals and determine that a stimulation signal should be generated and determine the stimulation signal characteristics such as pulse amplitude, pulse duration, pulse frequency, and pulse shape based on the analyzed EMG signal characteristics. The process can further include securing at least one electrode to a subject and delivering a stimulation signal to an area of interest in the subject. In this way, one or both VFs of a subject can be stimulated to perform at least one of treating glottic insufficiency or improving airway protection.

### EXAMPLES

To experimentally demonstrate the feasibility and advantages of the systems and methods provided herein, experimental LFES systems were created that included the architecture described above with respect to FIGS. 2 and 8.

### Example 1

### Overview

Disclosed herein are systems and methods for producing and using electrodes for LFES to restore glottic closure for improving voice and airway protection in cases of unilateral or bilateral VF paralysis. Specifically, a multi-contact electrode and method for electrode placement were developed for selectively stimulating target muscles in a way that provides balanced contraction forces for naturalistic VF closure. The electrode may comprise a modified epimysial paddle or strip design with multiple raised conductive contacts. As a proof-of-concept prototype, two custom-designed electrode strips were attached back-to-back so that current can be delivered in opposite directions and identified a way to implant these double-sided strip electrodes into the VFs so that the primary VF muscles can be independently stimulated for precise glottic closure. The capability of these electrodes to stimulate balanced glottic closure for intraoperative phonation has been demonstrated in a canine model of laryngeal paralysis, which may be achieved by a laryngeal pacing system using these new electrodes and implantation methods.

Further, the double-sided electrodes can serve both stimulation and recording functions when placed onto laryngeal muscles. In cases of unilateral VF paralysis, an electrode implanted within the healthy VF can detect EMG biopotentials associated with VF adduction. In cases where both VFs are paralyzed, closed-loop triggering can be accomplished by detection of EMG from vocal-related contractions of neck strap muscles or submental muscles (under the chin). An implanted processor can receive the EMG signals on the healthy side (or other neck locations) and use them to trigger FES through an electrode implanted in the paralyzed VF(s) to restore symmetrical VF movement and closure. In cases of CT paralysis, the FES electrodes can have an additional set of contacts oriented perpendicular to the double-layered strip described previously. Specifically, a vertically oriented strip attached to the lateral edge of the horizontally oriented double-sided strip can direct electrical current laterally towards the CT muscle, enabling the FES implant system to control vocal pitch in addition to VF adduction. In cases of unilateral CT paralysis, EMG from the healthy CT can be used to trigger and modulate stimulation of the paralyzed CT, as described previously for closed-loop stimulation of the TA and LCA muscles.

### Background

VF paralysis is typically caused by injury to one or both recurrent laryngeal nerve (RLN) branches of the vagus nerves innervating the VF closing muscles, e.g., the TA and LCA, and opening muscles, e.g., posterior cricoarytenoid (PCA). Vocal pitch is controlled mainly by contraction of the CT muscles, which pull the cricoid cartilage towards the thyroid cartilage, thereby stretching the VFs and increasing their frequency of vibration. Unlike all other laryngeal muscles that are innervated by the RLNs, the CT muscles are innervated by the SLNs, and CT paralysis can accompany VF paralysis if the SLN motor supply is disrupted. Further, without independent and balanced stimulation of specific laryngeal muscles, stimulated closure does not enable VF tissues to properly interact with the airstream from the lungs and thereby precludes normal VF vibration and phonation (voice). Further, conventional laryngeal pacing devices have only stimulated the PCA muscle for VF opening to facilitate breathing and have only used preset patterns of stimulation, i.e., open-loop control, rather than sensing and responding to the attempted muscle contractions of the patient, i.e., closed-loop control.

Detection of intended laryngeal contraction can be sensed using one or more sensing modalities from one or more body locations. Sensors detecting intended contraction can take the form of EMG electrodes attached to the skin surface superficial to target muscles or implanted within, i.e., intramuscular, or on, i.e., epimysial, target muscles as chronic implants. Multiple submental, e.g., under the chin, and ventral neck muscles contract simultaneously with voice production, making them an appropriate target for detecting the intention to produce voice. The amplitude of EMG signals from these muscles can be used to trigger LFES of paraly zed or paretic VFs to enable voice when EMG exceeds an activation or onset threshold, or when multiple recording locations simultaneously exceed independent thresholds. Likewise, termination of LFES can occur when EMG signals drop below one or more termination or offset thresholds. Having termination thresholds set lower than activation thresholds provides hysteresis that helps prevent rapid, inappropriate alternation of stimulation activation/termination when EMG activity levels during voice production vary near the activation threshold. Given that submental and ventral neck muscles contract at times when LFES of VF closure might not be appropriate, such as while yawning, EMG activity patterns detected from some muscles can also indicate when LFES should not occur. For example, vocal-related EMG can be ignored when there is simultaneous activity from muscles that indicate a non-vocal behavior is occurring.

In addition to EMG signals, triggering can be based on additional physiological or manual signals correlated with intended glottic closure. These signals can include neck surface vibrations detected by accelerometry, movements of speech articulators detected by implanted or surface markers distorting a magnetic field or visible markers, voice or speech sound acoustics via microphone(s), tracheal or esophageal pressure, bioimpedance of neck and thoracic tissue conformation, gyroscopes tracking head and neck movements, strain or bend gauges, ultrasonography, computer vision of head and neck movements, central or peripheral nervous system bioelectrical potential recordings, or a combination of these modalities. Individuals can also manually trigger their LFES-based glottic closure using a hand-held remote control or sensors on non-voice-related structures that they intentionally manipulate to initiate and terminate LFES. Such manual triggering can successfully close the glottis and protect the airway at the initiation of swallowing.

As discussed above, VF adduction and tension are achieved by contraction of the TA, LCA, and IA muscles. LFES to restore dynamic glottic adduction in cases of UVFP may require independent control of TA and LCA muscles. The IA muscle receives bilateral, overlapping innervation from the RLNs, thereby compensating for a unilateral loss of RLN function. Several TA/LCA muscle stimulation electrode designs have been tested with chronic VF implantation in dogs, including intramuscular cylindrical designs placed longitudinally within the VF, epimysial paddles placed on the lateral surface of the adjacent TA/LCA muscles, and paddles placed between the TA and LCA muscles.

In humans, there are usually 1-6 small branches from the RLN entering the LCA and 1-4 branches entering the TA, but the exact location of the branches are variable among individuals. Stimulation testing can be performed intraoperatively to determine which combination(s) of electrode contacts (for bipolar or tripolar current delivery) on each paddle side deliver the most effective muscle stimulation. The electrode contact pairings and all other stimulation parameters can also be adjusted in the clinic with a patient awake and producing voice and swallows to achieve optimal voice and swallowing function. Tuning the voice post-operatively, after inflammation related to surgical tissue trauma has resolved, is expected to provide a better voice outcome compared to intraoperative voice tuning when placing paraglottic materials and better than VF positioning based on presurgical imaging and intraoperative visual feedback of static VF position during material implantation.

### Methods—Electrode Manufacturing

It is contemplated that an electrode, e.g., a paddle electrode, can have a variety of different configurations according to the present disclosure. For example, an electrode may be a generally rectangular electrode with a plurality of contacts disposed thereon that are axially aligned with one another, as discussed above with respect to FIGS. 7A-7C. In other examples, an electrode can include multiple branches or arms to provide detection and/or stimulation flexibility once implanted in an area of interest of a subject. For example, an electrode can include a first paddle that has a first plurality of disc contacts disposed thereon, and a second paddle that has a second plurality of disc contacts disposed thereon. Further, the electrode may be bifurcated into a first arm and a second arm, such that the first paddle can be coupled to a first side of the first arm and the second paddle can be coupled to a second side of the first arm. In other words, the first paddle and the second paddle can be oriented back-to-back, i.e., disposed on opposite sides of, the first arm such that each paddle's plurality of electrode contacts face in opposite directions in order to stimulate different target muscles, e. g., a first target muscle and a second target muscle. In addition, the electrode can further include a third paddle having a third plurality of disc contacts, and the third paddle can be coupled to the second arm in order to stimulate yet a third target muscle. In particular, the first arm may be configured to face and/or contact the TA and/or LCA muscles of a subject, while the second arm may be configured to face and/or contact the CT muscle of a subject, as discussed below. In other examples, an electrode can include more than two arms and/or more than two paddles.

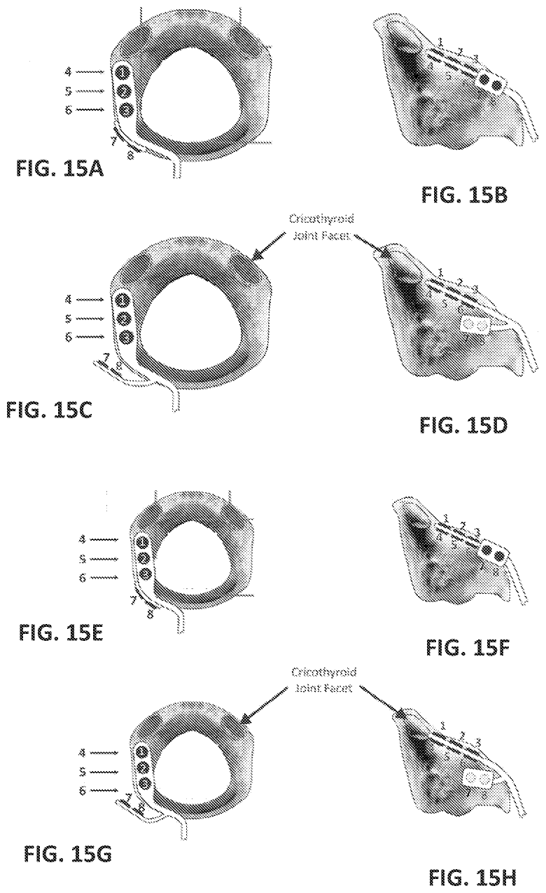

The paddle electrodes used in the present example were custom fabricated by PMT to predetermined specifications. In particular, a series of three and four platinum disc contacts, 2 mm in diameter separated 4 mm center-to-center, on 4 mm-wide silicone paddles were fabricated by PMT (see FIGS. 7A-7C) and then fused back-to-back for directing current in opposite directions, e.g., towards the TA vs. LCA. Two-contact paddles were also made for directing current, or recording EMG potentials, towards the CT muscle (see FIGS. 15A-15H). The CT-facing contacts can reside on the lateral surface of the TA/LCA double-sided paddle, falling perpendicular to the paddle containing contacts for the TA and LCA muscles and oriented to contact the CT on its internal surface (see FIGS. 15A, 15B, 15E, 15F), or the contacts can reside on an independent paddle tethered by a short distance of cable so that it can be placed outside of the laryngeal framework and contact the CT on its superficial surface (see FIGS. 15C, 15D, 15G, 15H).

The double-sided design was rigid enough to be pushed to depth in the bluntly dissected pathway between the TA and LCA in the in vivo dog testing and cadaveric dog and human larynx testing. However, an additional fabrication option would be to use flexible and stretchable electrodes 3D printed using a combination of conductive and non-conductive silicones. A highly flexible electrode can decrease tissue trauma and can withstand over 100 million flex cycles without breaking, although highly flexible electrodes may also lack sufficient rigidity for unsupported insertion. Achieving flexible electrode implantation can be done using micro alligator ear forceps or by making electrodes with a slightly raised distal ridge or lip against which a U-shaped stylet can be pushed to guide the paddle into position. The U-shape of the wire will not block the electrode contacts when functionally confirming electrode position. Withdrawing the stylet will release it from the distal ridge, leaving the electrode behind (due to tissue/paddle adherence cause by capillary forces and wetting effects). The lip will also act as a barb to hold the electrode in place after confirmed functional placement, yet will be flexible enough to enable retraction of the electrode without tissue damage or electrode damage should the electrode need repositioning or removal. The viability of this stylet-delivery approach has been confirmed with flexible silicone electrodes inserted into unfixed cadaveric dog and human larynges, and prior work indicates that highly flexible Gore-Tex (PTFE) strips can be pushed under the inferior margin of the thyroid cartilage in a similar manner.

The initial paddle electrode design was based on measurements taken in excised dog and human larynges, in addition to literature providing dimensions of the human larynx paraglottic anatomy. Given the similarities in larynx size and relative positions of the TA and LCA in dogs and humans, a design that works well for stimulating phonatory adduction in one would likely work well in the other. Nevertheless, average human male and female anatomy was used when designing the electrodes used in initial dog LFES experiments, as illustrated in FIGS. 15A-15H. Specifically, electrodes have been placed in three adult male cadaveric larynxes (see FIGS. 15A-15D and three adult female cadaveric larynges (see FIGS. 15E-15H) to confirm that the dimensions described herein are appropriate for human LFES. During this confirmation, the following were measured: 1) the depth for electrode insertion from the inferior margin of the thyroid cartilage at the lateral cricothyroid ligament to the anterior arytenoid surface between the TA and LCA (for electrode length); and 2) the horizontal distance from the lateral surface of the TA/LCA to the lateral subglottic wall at anterior, mid-muscle, and posterior locations, for paddle width. Unlike humans, dogs lack sexual dimorphism in larynx size, and vocal function (when controlling for body weight), so using only female dogs in the initial animal experiments was appropriate. These data guided the dimensions of the new electrode described herein and identified a range of sizes needed for adult humans. Further, the location of the RLN branches entering the TA and LCA muscles were noted to determine the number, location, size, and inter-contact distance of conductive contacts on the respective surfaces of the double-sided paddles. In pilot testing with two canine models, the distal-most contacts facing upward or downward provided excellent selectivity for TA and LCA stimulation, respectively. LFES in dog testing demonstrated controlled VF movements and phonatory glottic closure caused by selective TA and LCA stimulation.

### Methods—Electrode Implantation in Canine Models

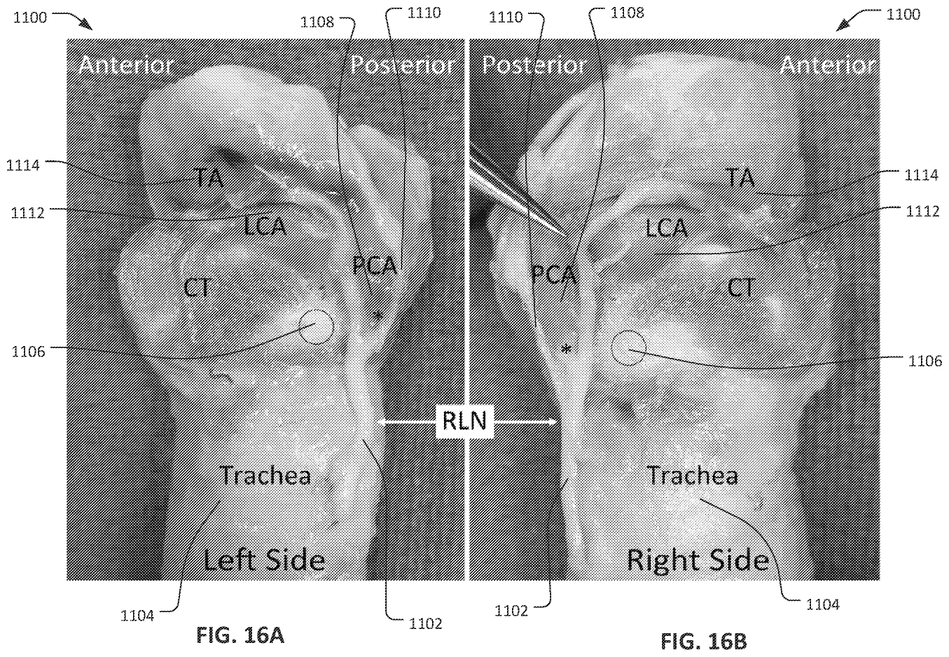

FIGS. 16A and 16B illustrate a dog larynx 1100 after removal of thyroid cartilage and most of the CT muscle. As shown, the RLN 1102 travels along a lateral posterior edge of the trachea 1104, anterior to the esophagus (not shown) and passing posterior to the cricoarytenoid joint 1106. Branches of the RLN 1102 enter the PCA 1108 and pass posterior to the PCA 1108 to join sensory fibers at the superior laryngeal nerve 1110. After the RLN 1102 passes superior to the cricoarytenoid joint 1106, branches thereof innervate the LCA 1112 and TA 1114. This RLN innervation pattern and laryngeal anatomy is very similar to the adult human. An electrode placed lateral to the LCA 1112 and TA 1114 in the paraglottic space would likely stimulate all incoming motor innervation and may result in reduced glottic closure due to indiscriminate LCA and TA activation. In contrast, a double-sided electrode placed in the horizontal connective tissue plane between the LCA 1112 and TA 1114 muscles would remain medial to the incoming nerve supply and would only stimulate RLN branches that enter the LCA 1112, e.g., for inferiorly facing electrode contacts, or TA 1114, e.g., for superiorly facing electrode contacts.

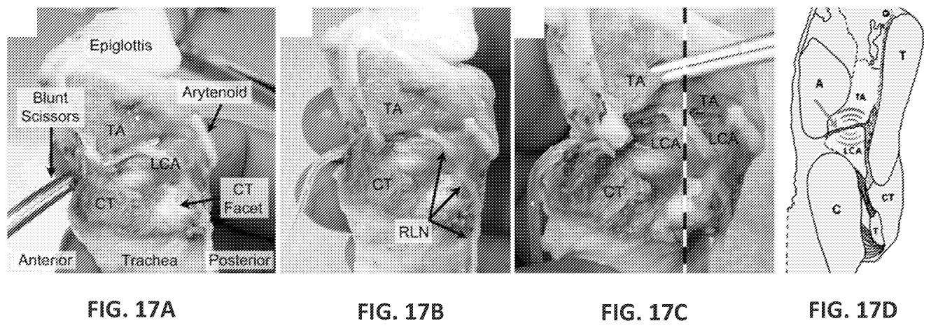

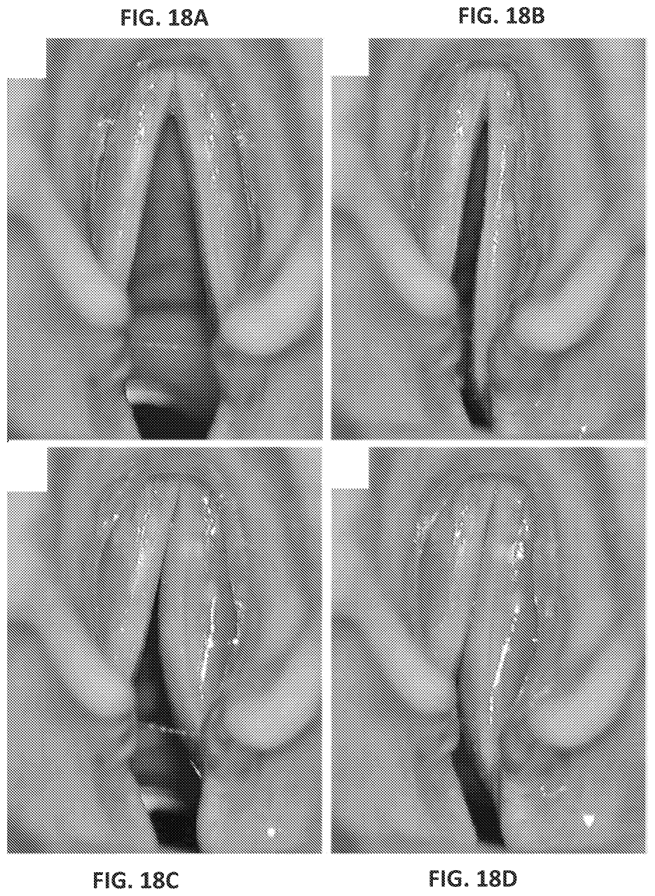

Further, two dogs underwent surgery with LFES system testing in conjunction with suspension microlaryngoscopy under I.V. general anesthesia with spontaneous breathing. The glottis was viewed with a surgical microscope fitted with a high-definition camera, high-speed-video camera, and audio recorder. At the initial surgery for each dog, a bipolar silicone nerve-cuff electrode was implanted on one RLN and a multi-contact, double-sided paddle electrode was implanted into each VF through a single neck incision. After confirming stimulation electrode position effectiveness for achieving selective TA and LCA stimulation, the electrodes were sutured onto the anterior cricoid using 3-0 Prolene. Suturing or otherwise anchoring the electrode cables to the cricoid cartilage minimizes repetitive electrode flexion and prevents the electrode(s) from being dislodged from the implantation site with neck movements. This would be done with the electrode implantations demonstrated in FIGS. 17A-17D if placement was intended to be chronic/permanent, but these illustrations do not demonstrate anchoring sutures affixing the electrode to the anterior cricoid. Similar electrode anchoring with non-resorbable suture, e.g., 3-0 Prolene, was performed. Specifically, FIGS. 18A-18D illustrate a series of detailed views of canine model VF adduction and abduction as a result of independent and unilateral TA and LCA muscle stimulation while under general anesthesia and suspension microlaryngoscopy. Further, FIG. 18A illustrates the left and right VFs at rest, i.e., without any stimulation. FIG. 18B illustrates the VFs being adducted during right LCA stimulation, FIG. 18C illustrates the VFs being adducted during right TA stimulation, and FIG. 18D illustrates VFs being adducted during both right LCA stimulation and right TA stimulation. As shown, simultaneous LCA and TA stimulation was found to elicit the most dramatic VF adduction in comparison to only LCA stimulation or only TA stimulation. Stimulation of the RLN was performed using a double-sided paddle electrode to deliver pulse trains in the range of 20 -120 Hz, 0.2-1 ms pulse width, and 0.01-2 mA. The VF contraction elicited via unilateral RLN stimulation emulated volitional VF adduction by a person during voice production, the EMG from which acted as the trigger for closed-loop stimulation of the contralateral VF. EMG-triggered stimulation of the contralateral VF was performed with pulse trains in the range of 20 -120 Hz, 0.2-2 ms pulse width, and 0.01-12 mA.

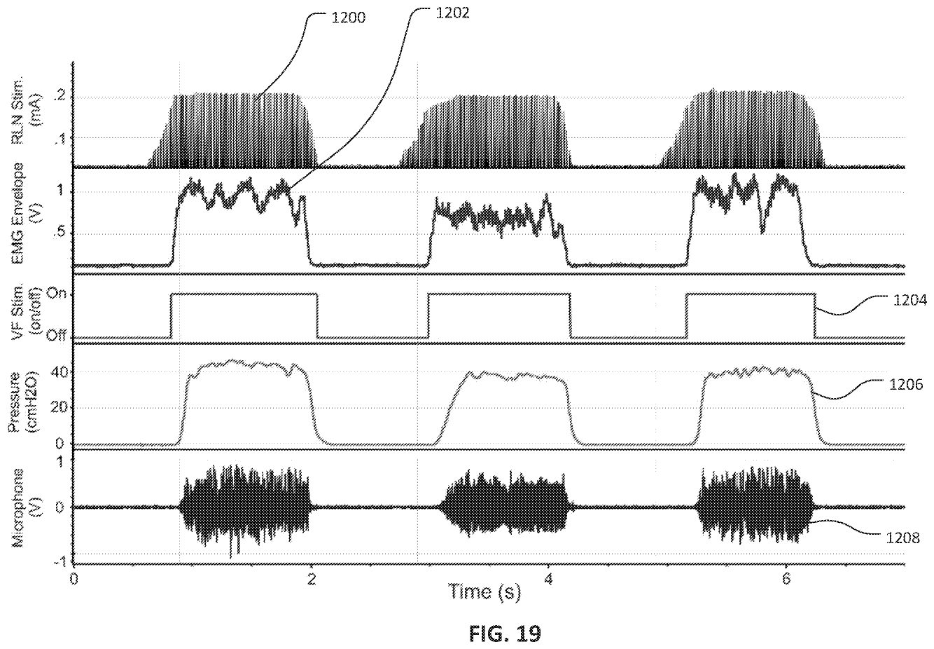

Referring now to FIG. 19, phonation testing was performed by exposing the ventral trachea through a midline neck incision, and intubation tubes were inserted towards the lungs and larynx to provide optimal control of airflow for ventilation and phonation, respectively. Heated and humidified air was passed below the glottis while the VFs were adducted through electrical stimulation of laryngeal adductor muscles or the RLN. A pressure probe was also placed in the trachea to measure subglottic pressure. Stimulation of bilateral VF adduction was triggered manually during initial phonation testing. As illustrated in FIG. 19, three phonations during closed-loop stimulation phonation were recorded. Specifically, the left RLN was stimulated with 1 s, 80 Hz pulse trains 1200 causing the left VF to adduct and generate LEMG 1202 recorded from the left VF as it adducted. The LFES system detected the LEMG 1202 recorded from the left VF and triggered simultaneous right VF stimulation 1204, thereby bilaterally closing the glottis, and building subglottic pressure 1206, which caused vibration/phonation 1208 from the adducted VFs.

Acoustic and VF vibratory assessment of phonation was performed on manually triggered phonation rather than closure triggered by RLN stimulation because the latter did not have symmetrical right/left VF postures due to antagonistic abduction from the PCA muscle coinciding with the TA/LCA adduction. Further, selective unilateral PCA denervation was done towards the end of intraoperative testing for more natural RLN-stimulated adduction. After initial closed-loop phonation testing, the RLN branch to the PCA was cut, and the closed-loop testing was repeated as triggered by RLN stimulation. Cutting the PCA branch was done as a final step to mitigate risk of unintentional TA/LCA nerve branch injury, which did not occur. At the completion of testing, electrode position was documented at post-mortem inspection.

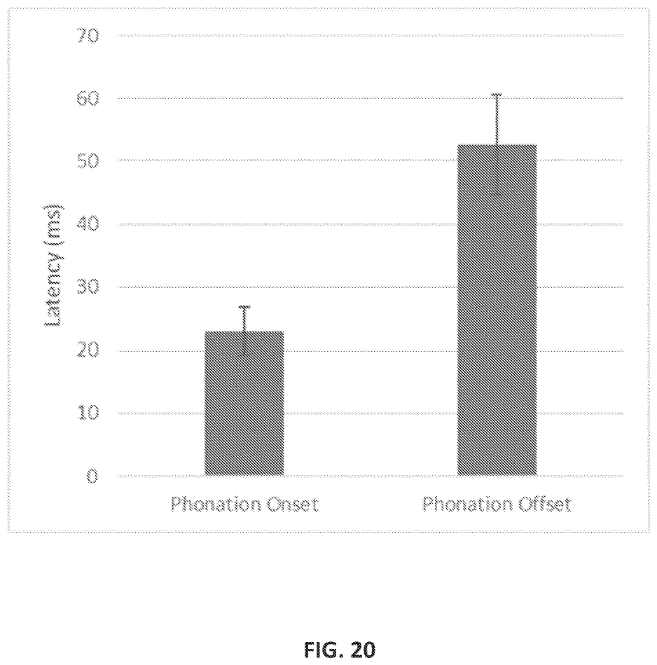

To assess closed-loop triggering and phonation, the synchrony between nerve-cuff-elicited muscle contraction and the closed-loop-triggering of VF muscle stimulation was measured as onset (i.e., activation) and offset (i.e., termination) latencies, as illustrated in FIG. 20. Threshold adjustment in relation to stimulation responses minimized latency errors while avoiding stimulation drop-outs during intended phonation, which has been performed in prior experiments with neck surface LEMG-triggering of a voice prosthesis in human patients. Closed-loop LFES of bilateral VF adduction was achieved without stimulation drop-out, and with onset (i.e., activation) and offset (i.e., termination) latencies of less than 30 ms and 60 ms (respectfully) triggered by RLN-elicited LEMG from the contralateral VF. This finding was similar to the near-simultaneous, closed-loop stimulation of bilateral adduction that has been achieved in recent canine model experiments and that may be adequately rapid for LFES reanimation of bilateral glottic closure in human patients.

### Methods—Electrode Implantation in Human Cadaveric Larynxes

The incoming motor nerve supply to the TA and LCA muscles is lateral to a double-sided paddle electrode placed horizontally between the TA and LCA muscles such that the nerves to each muscle are not stimulated until entering their respective muscle, avoiding indiscriminate stimulation of all incoming motor axons from the RLN. This new electrode design has been implanted and tested in two live dogs and six human cadaver larynges, and it has been found that electrode placement is straightforward and minimally invasive.

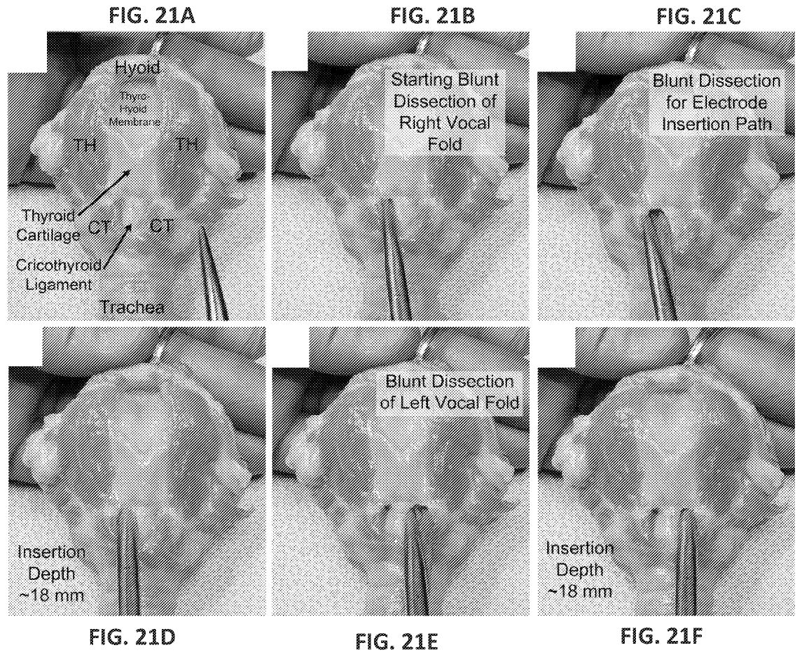

The only tissue cutting needed for electrode implantation is a standard horizontal skin incision to expose the anterior larynx at the large gap between the thyroid and arytenoid cartilages. To accomplish this, and as illustrated in FIGS. 21A-21F, narrow, blunt-tipped scissors are then introduced at the margin between the medial edge of the CT muscle and the lateral cricothyroid ligament and carefully advanced in the anterior-posterior direction until reaching the arytenoid cartilage at a depth of approximately 18-22 mm for males and 16-18 mm for females. Put another way, a blunt dissection of a VF is performed to create a cavity in which to insert an electrode, as discussed below. This path of blunt dissection falls along a natural plane of intermuscular fascia separating TA and LCA in dogs and humans. Dissection can be guided by watching the slight medial distension of the durable, subglottic conus elasticus, ensuring that the plane falls between the TA muscle, superiorly, and the LCA muscle, inferiorly, and that dissection does not veer laterally towards the thyroid cartilage which may indiscriminately stimulate the incoming LCA and TA nerve supplies.

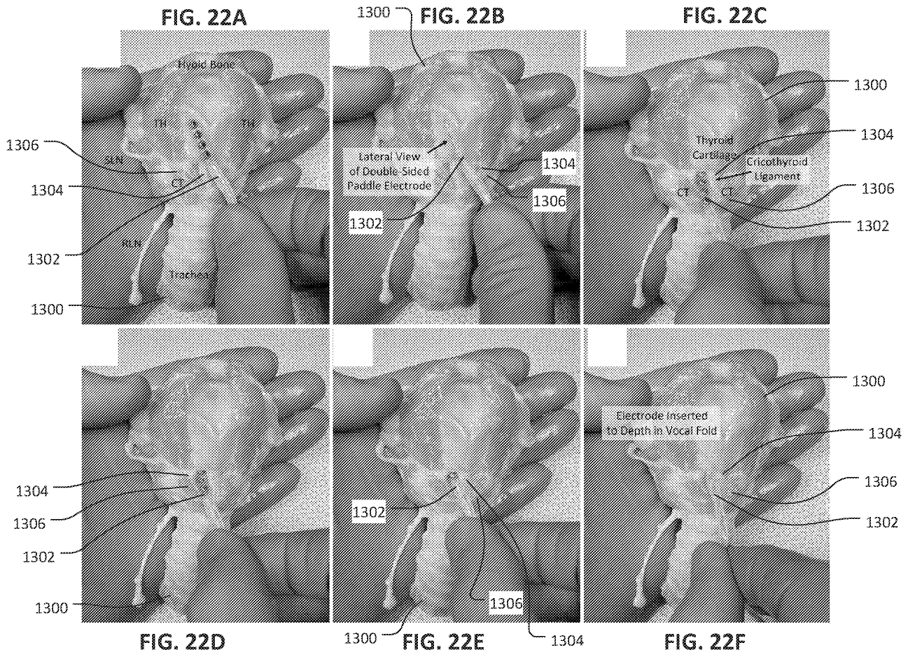

Electrode pathway blunt dissection and insertion according to the present disclosure may not involve cartilage removal or other invasive tissue cutting. This is in stark contrast with conventional ML/AA surgery which involves cutting the thyrohyoid muscle and sawing a window through the thyroid cartilage for anterior VF implant placement (ML) and cutting pharyngeal constrictor muscle to permanently immobilize the cricoarytenoid joint when suturing the arytenoid into a phonatory position during AA. As illustrated in FIGS. 22A-26F, proper electrode position is typically achieved on initial insertion because the flat paddle inherently falls between the TA and LCA muscles when placed in the transverse connective tissue plane. Electrode position can be verified and refined during placement by visibly unambiguous activation of the TA and LCA muscles when electrical stimulation is passed through the superior and inferior paddle contacts, respectively.

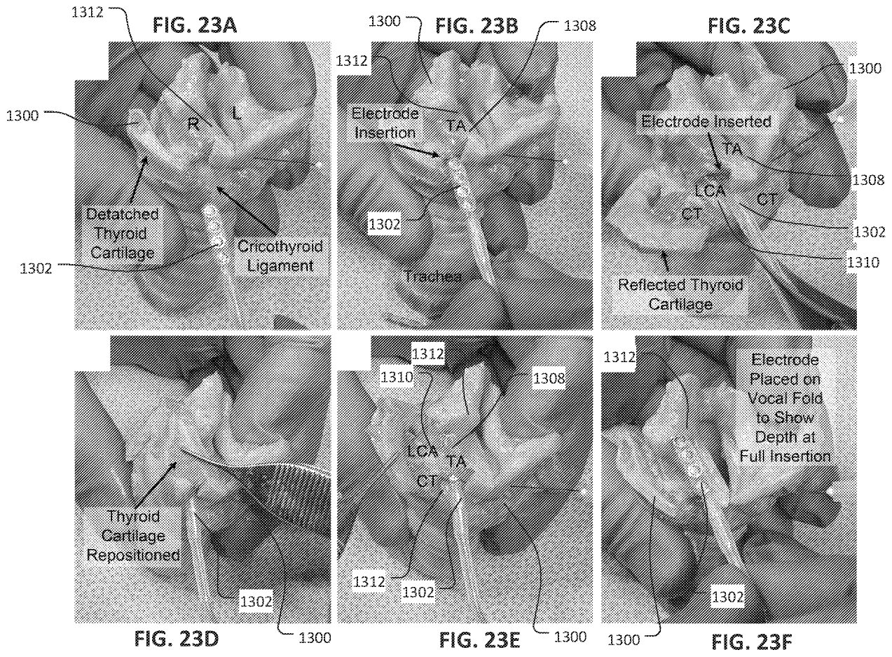

Specifically referring to FIGS. 22A-22F, double-sided paddle electrode insertion is illustrated for a female cadaveric larynx 1300. A paddle electrode 1302 is inserted lateral to a CT ligament 1304 and medial to a CT muscle 1306 before being advanced into the right VF (not shown) to a depth of approximately 18 mm. To further visualize paddle electrode 1302 insertion, reference is made to FIGS. 23A-23F which depict the cadaveric larynx 1300 after removal of the superior third of the right side of the thyroid cartilage and the false VF. Specifically, the paddle electrode 1302 is shown inferior to the TA muscle 1308 and superior to the LCA muscle 1310 after insertion, and the paddle electrode 1302 is placed on the right VF 1312 to indicate the contact locations along the VF length when inserted. Thus, it will be understood that the paddle electrode 1302 is configured to be inserted, i.e., placed or located, between a subject's TA muscle and LCA muscle during implantation.

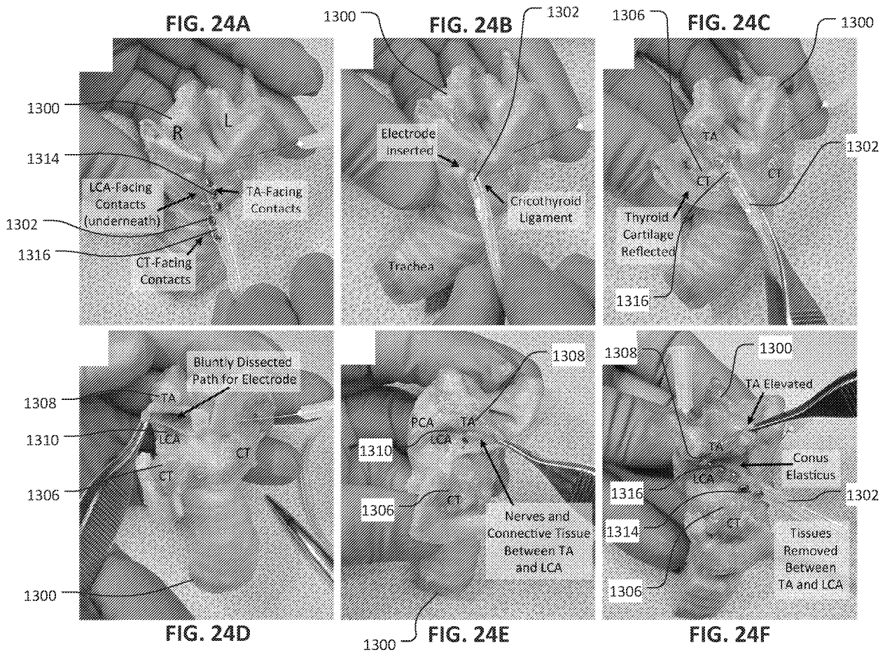

With additional reference to FIGS. 24A-24F, the cadaveric larynx 1300 is illustrated after vertical incision of the right thyroid cartilage and detachment from the CT joint. As shown, the paddle electrode 1302 includes three TA-facing contacts 1314, three LCA-facing contacts (not shown), and two CT-facing contacts 1316. In some aspects, the TA-facing contacts 1314 and the two CT-facing contacts 1316 are on separate arms of the paddle electrode 1302, as will be discussed below in greater detail. Further, the CT-facing contacts 1316 will contact the CT muscle 1306 when implanted (see FIG. 24B).

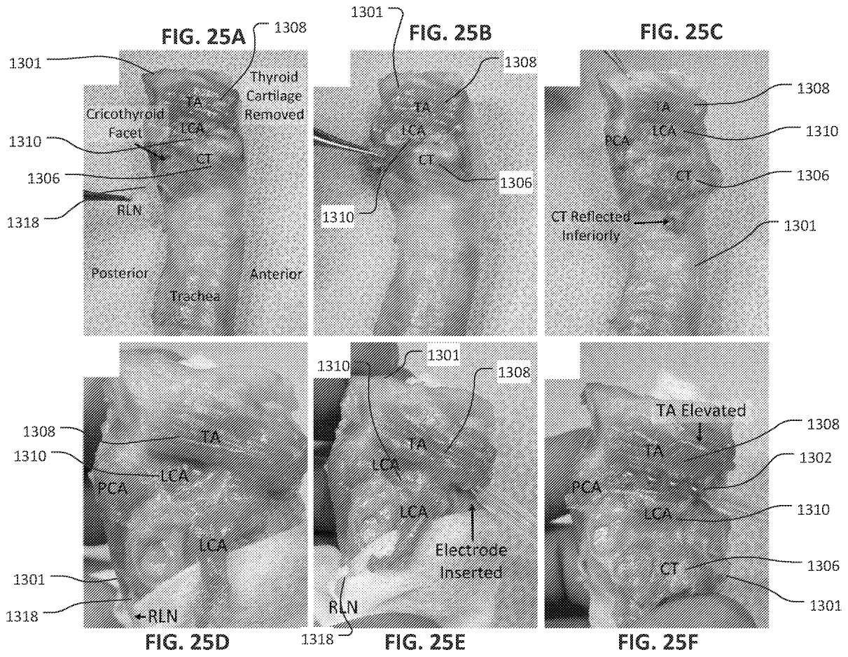

Referring now to FIGS. 25A-25F, lateral views are illustrated of a male cadaveric larynx 1301 after removal of the thyroid cartilage to expose paraglottic muscles. As illustrated, the RLN 1318 passes posterior to the CT joint's facet before sending branches to the TA muscle 1308 and LCA muscle 1310. The CT muscle 1306 attaches to the lateral surface of the cricoid cartilage, which is best visualized after detachment from the thyroid cartilage and reflecting the CT muscle 1306 inferiorly. The LCA muscle 1310 has a relatively small lateral muscle belly under which the RLN 1318 typically travels. After detachment of this small LCA belly from the muscular process of the arytenoid and reflecting it inferiorly, it exposes the multiple branches of the RLN 1318 that enter the TA muscle 1308 and LCA muscle 1310. The incoming RLN 1318 nerve supply remains lateral to the double-sided paddle electrode 1302, thereby facilitating selective stimulation of these branches after they have entered the TA and LCA muscles 1308, 1310. After dissection of the tissues between the TA and LCA muscles 1308, 1310, elevation of the TA muscle 1308 with forceps, and placement of the paddle electrode 1302, as illustrated in FIG. 25F, electrode contacts can be seen facing their target muscles, i.e., the CT muscle 1306, the TA muscle 1308, and/or the LCA muscle 1310.

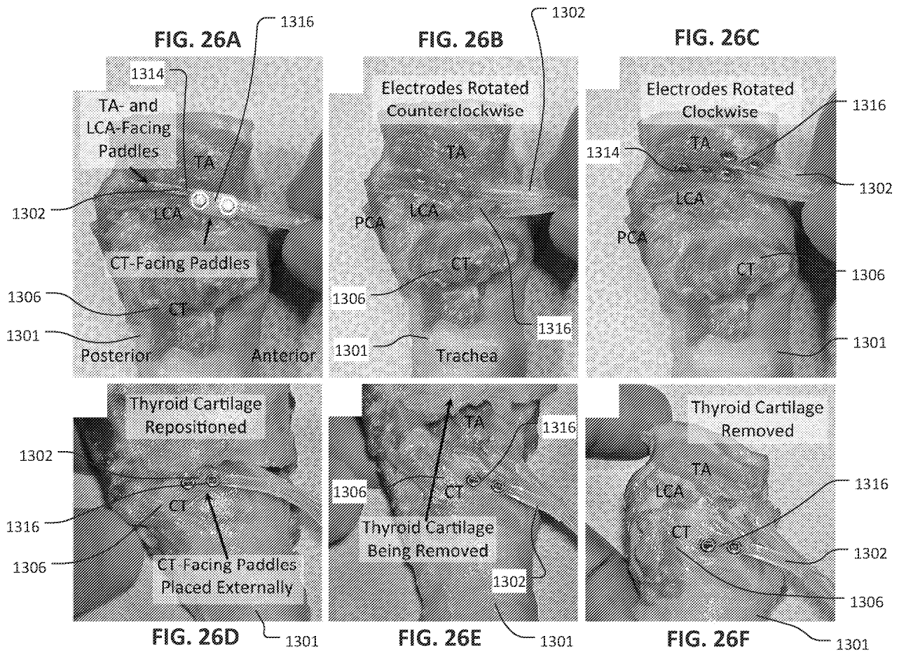

Referring now to FIGS. 26A-26F, additional lateral views of the cadaveric larynx 1301 are illustrated after removal of the thyroid cartilage to expose the paraglottic muscles. Additionally, FIGS. 26A-26F depict the paddle electrode 1302 with separate TA-facing contacts 1314 and CT-facing contacts 1316, similar to those shown in FIGS. 20A-20F. As discussed above, the paddle portion containing the CT-facing contacts 1316 can be independent from the paddle portion containing the TA-facing contacts 1314 and/or LCA-facing contacts. Specifically, the CT-facing contacts 1316 can be coupled to the other contacts by a short length of cable, and the CT-facing contacts 1316 can face laterally to stimulate or record from the CT from its deep surface (see FIGS. 26A-26C) or face medially to stimulate or record from the superficial surface of the CT muscle 1306, as illustrated in FIGS. 26D-26F. In the illustrated non-limiting example, the mobilized thyroid cartilage is repositioned in FIG. 26D to demonstrate that the CT-facing contacts 1316 can be located external with respect to the laryngeal skeleton.

### Discussion

The in vivo dog experiments demonstrated independent control of TA and LCA muscle stimulation. Electrical pulse-trains directed towards the TA versus LCA caused an unambiguous change in glottic configuration, consistent with prior detailed descriptions of TA versus LCA stimulation through electrical stimulation of exposed RLN nerve branches. However, conventional techniques have yet to identify an electrode design capable of selective TA and LCA stimulation. When using the electrode design described in this example, TA stimulation caused measurable VF shortening with bulged (convex) adduction of the anterior glottis (see FIG. 18C), whereas LCA stimulation adducted the arytenoid vocal process, lengthened the VF, and caused a straight medial edge with anterior and posterior glottic closure (see FIG. 18B). Furthermore, modulation of steady-state phonations has been demonstrated via independent TA and LCA stimulation. Phonation quality was assessed by acoustic and HSV analysis. Vibration periodicity in relation to combinations of LCA and TA stimulation parameters was measured from 1) audio recordings and 2) HSV-based measures of visible glottal closure. LCA stimulation alone caused adduction capable of phonation but with a breathy quality, whereas adding graded levels of VF tension with TA activation decrease breathiness, increased mid-VF glottic closure, and reduced VF lateral excursion during phonation. Graded control of TA and LCA stimulation in relation to varying LEMG levels, e.g., proportional control, will provide optimal dynamic restoration of VF adduction in relation to contralateral VF degrees of activation and vocal effort.

The LFES system described herein represents a novel and substantially different laryngeal pacing approach intended to restore voice and provide protective glottic closure by detecting the occurrence or intention of laryngeal contractions and electrically stimulating paralyzed muscles to achieve the desired action. In recent studies using a dog model of VF paralysis, closed-loop LFES stimulation of symmetrical closure was achieved by using LEMG activity from the healthy VF to trigger stimulation of the paralyzed VF. As expected, the balance between lateral cricoarytenoid (LCA) and thyroarytenoid (TA) muscle stimulation was observed to be significant for optimizing phonatory adduction. Therefore, independent control of LCA and TA stimulation is an advantageous feature of the disclosed multi-contact electrode and LFES system design. Although paralysis of the CT muscle, which is innervated via the SLN, is less common than paralysis of the RLN-innervated muscles, there are instances where stimulation of the CT can restore vocal pitch range. Therefore, CT stimulation and recording contacts are beneficial to have as an option in the plurality of multi-contact laryngeal electrode designs and LFES system configurations.

Further, electrodes placed within the VF are effective for TA stimulation but may not facilitate phonation due to excessive tension/bulging of the mid and anterior regions of the glottis. Paddle electrodes placed lateral to the TA/LCA in the lateral paraglottic space tend to stimulate all incoming recurrent laryngeal nerve (RLN) branches, causing indiscriminate TA and LCA muscle activation. The TA and LCA muscles are immediately adjacent in the VF, separated by an intermuscular fascia. The example described herein demonstrates that placing double-sided multi-contact paddles horizontally between the TA and LCA muscles within the fascial plane (see FIGS. 17B-17D) allows selective control over these muscles, which is significant for stimulating closure that supports phonation.

### Example 2

### Overview

Laryngeal paralysis can impair voice, respiration, and deglutition—particularly when VF neural dysfunction is bilateral. The impact of laryngeal nerve damage has been known since Galen's observations in the second century AD, and for the past four decades, researchers have studied the potential for using functional electrical stimulation (FES) to restore dynamic VF abduction and adduction for breathing and swallowing, respectively. Evidence has also been presented that electrical stimulation may have more long-term and beneficial biologic effects, such as enhancing or altering the patterns of reinnervation and preventing and/or rejuvenating muscle atrophy associated with laryngeal nerve injury or advanced age.

In the context of stimulation applied to aid a rhythmic process such as abduction for respiration, the term laryngeal pacing has been commonly applied. Implanted laryngeal pacers have proven effective for restoring respiration capabilities in animal models of bilateral laryngeal paralysis and in limited human trials. These studies show that periodic stimulation of VF abduction to facilitate respiration can be done in an “open-loop” manner, e.g., using preset patterns. However, to restore VF adduction for swallowing or phonation, devices will need precise user or manual control of FES activation or function in a “closed-loop” manner whereby the implant responds to one or more physiological signals for triggering FES events. Closed-loop stimulation is more efficient, which is important for battery-powered devices, and enables the implant to respond to the biological or functional needs of the individual better than preset patterns. For example, in the neuromodulation field, closed-loop stimulation has been used to trigger deep brain stimulation when seizure activity is detected. There are also several closed-loop FES systems that restore function through skeletal muscle stimulation. For example, foot-drop can be corrected by external or implantable FES devices that stimulate foot dorsiflexion in response to afferent nerve activity e.g., at heel contact or leg swing detection.

Closed-loop FES for unilateral laryngeal reanimation should be feasible due to the bilateral symmetry typically observed in VF motion, whereby activity detected in the healthy side can be used to control patterns of FES in the paralyzed side. In cases of bilateral laryngeal paralysis, other physiological signals should be appropriate to trigger FES, such as EMG from neck strap muscles, CT, diaphragm, or mechanical detection of respiration-related movements as these contractions and/or movements are correlated with activity of intrinsic laryngeal muscles.

The goal of the present example has been to work toward a clinical FES device that can be used for closed-loop laryngeal stimulation for VF adduction to improve protective glottic closure and phonatory function. In doing so, the strategy discussed herein has been to use animal-based experiments as part of an iterative development process where custom-built prototype devices can be tested under conditions that mimic unilateral or bilateral paralysis with attending atrophy and/or synkinetic reinnervation. Here, the acute and long-term experiments in canines are described, testing several iterations of two main design approaches to explore the ability of the units to function over time after implantation, the efficacy of the stimulation to alter vocal fold position and/or tension and thereby affect phonation, and stimulation amplitudes required to animate healthy versus neuropathic vocal folds in the context of different epimysial and intramuscular electrode configurations. These experiments can help establish specifications for a clinical device, and practical considerations are discussed as they relate to LFES that merit further investigation.

### Methods—Electrode Implantation

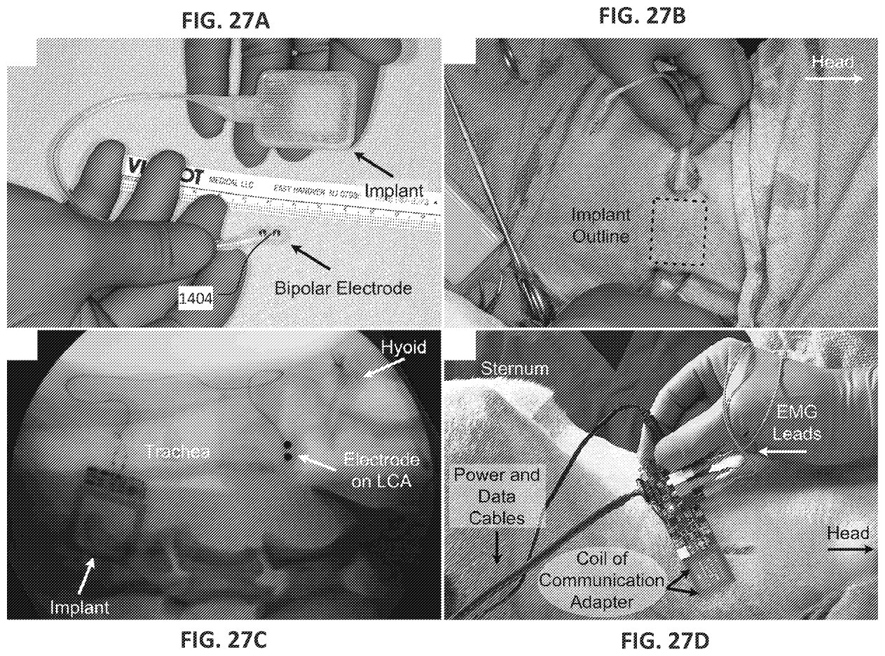

Two principle implant design pathways were followed that differed primarily in implant power supply. One design is an inductively powered laryngeal stimulator (ILS), as illustrated in FIGS. 27A-27D that comprises an external communication adapter, or transmitter, placed in close proximity to an implant, or receiver. An electrical field generated by the external unit powers the implant through the skin and provides two-way communication between the external and internal devices. The second design pathway likewise has external and internal components, but the implant is battery-powered (BLS) and can operate independently from the external device once it receives instructions through an infrared communication channel transmitted through the skin. The latest iteration of each design is capable of recording an EMG signal, from the external unit of the ILS or implanted unit of the BLS, and triggering a stimulation pulse train from the implanted unit when EMG magnitude exceeds a predetermined threshold.

Five adult female hounds weighing 21 to 30 kg were used for this study. Two animals had intact laryngeal innervation when receiving chronic ILS implantation for periodic testing. The other three animals were tested using nonimplanted versions of the BLS, e.g., tested in acute experiments. The ILS was implanted for long-term evaluation in two dogs. As illustrated in FIGS. 27B and 27C, the implant was placed in the left lateral neck in each animal, and a bipolar stimulation electrode was positioned on the epimysium of the right (dog No. 1) or left (dog No. 2) lateral cricoarytenoid (LCA) muscle. The animals were under general anesthesia with spontaneous respiration, and suspension microlaryngoscopy was used for the device implantation surgery and periodic device testing.

The animals first received 0.01 mg/kg glycopyrrolate, 0.05 mg/kg acepromazine, and 0.1 mg/kg butorphanol intramuscularly to provide partial sedation and analgesia. An intravenous (IV) line was placed in the forelimb, and Propofol was used to induce (4-6 mg/kg IV to effect) and maintain (0.3-0.5 mg/kg/min IV to effect) anesthesia. Single doses of an antibiotic, cefazolin, 25 mg/kg, and an anti-inflammatory, dexamethasone, 0.2 mg/kg, were also given via IV before surgery.

The silicone or epoxy-encapsulated ILS circuit board of the implant was placed into a subcutaneous pocket posterolateral to the external jugular vein, as depicted in FIG. 27B, and the stimulation electrode was tunneled to an incision near the larynx. The electrode was then advanced posteriorly under the inferior edge of the thyroid lamina. After confirming that stimulation produced robust ipsilateral VF adduction consistent with LCA contact, the electrode was sutured to the cricoid cartilage with 3-0 Prolene, as illustrated in FIG. 27C. Deep tissue planes were reapproximated using 3-0 Vicryl sutures, and incisions were closed with interrupted 2-0 or 3-0 Prolene sutures.

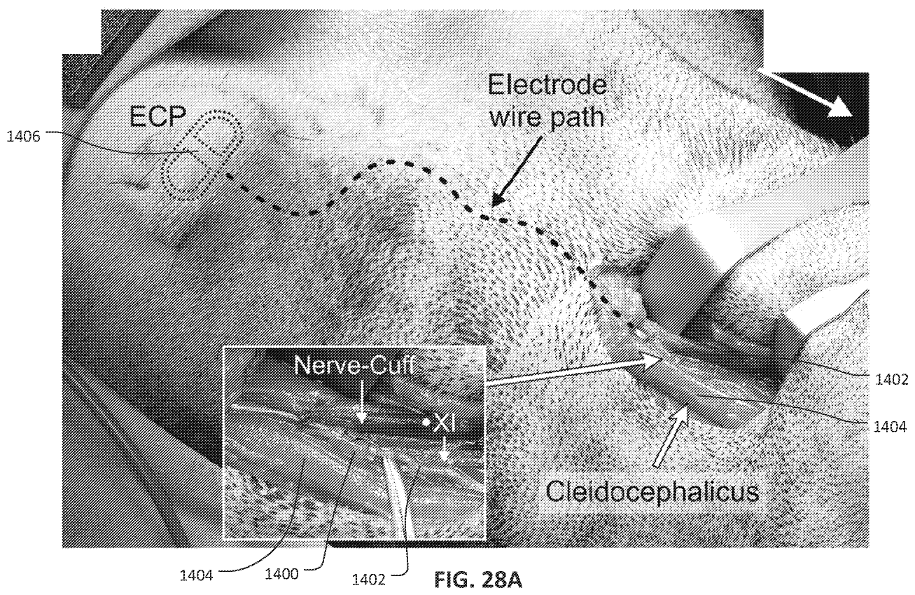

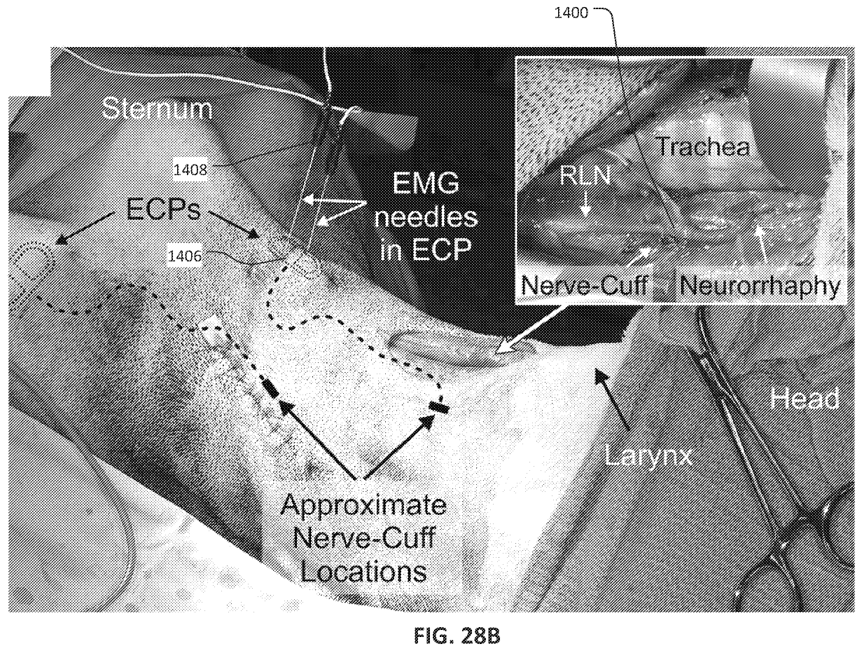

Closed-loop stimulation in response to an EMG triggering signal was achieved by permanently implanting a nerve cuff electrode 1400 onto a branch of the spinal accessory nerve 1402 in one side of the neck supplying the cleidocephalicus (CC) muscle 1404, as illustrated in FIGS. 28A and 28B. This experimentally elicited contraction of the CC muscle 1404 was used to simulate the natural co-contraction of neck strap muscles that occurs during human voice/speech. This muscle is comprised of parallel cleidocervicalis and cleidomastoideus bellies and is found just inferolateral to the external jugular vein. It was chosen as the EMG source for closed-loop stimulation because it represents a neck muscle that is close enough to the larynx to evaluate laryngeal stimulation current spread, yet it does not directly connect to the hyoid bone or laryngeal framework and therefore does not cause appreciable laryngeal movement upon contraction. Each cuff electrode 1400 contained a pair of 100 μm platinum wires spaced 2 mm apart running circumferentially within a 1 mm internal diameter silicone tube. Bipolar cuff electrode leads terminated in a custom-made, 2-contact electrical connection port (ECP) 1406, allowing for periodic connection using insulated needle electrodes 1408 inserted through the overlying skin and into the ECP's conductive contacts.

With continued reference to FIG. 28A, the nerve cuff implant location on the spinal accessory nerve 1402 supplying the CC muscle 1404 was reached through a 6-8 cm skin incision posterolateral to the external jugular vein approximately midway between the hyoid and sternum. After a cuff electrode 1400 was secured on the nerve 1402 with three cuff-closing sutures, the wire leads were secured to the CC with non-resorbable sutures, and a tunnel was created with blunt dissection below the skin to provide a location for the ECP 1406 that was not directly beneath an incision. The incision was then closed as previously discussed.

### Methods In Vivo Testing of ILS

Closed-loop stimulation of unilateral VF adduction with the ILS was performed at the time of implantation and four additional times. For periodic ILS testing, dogs were anesthetized as described previously, and suspension laryngoscopy was performed with a modified univalve speculum and viewed with a surgical microscope fitted with a high-definition camera and a high-speed video camera. Video of the surgical microscope view was digitized at 30 fps at a resolution of 1920×1088 pixels, with audio obtained from a ceiling microphone and amplifier at 48 kHz as well as a portable audio recorder placed near the animal's head. Color high-speed video was also collected at 4000 fps at a resolution of 320×512 pixels during phonation testing to capture VF motion, as discussed below. Further, an overhead camera recorded a 1920×1088 pixels, 30 fps view of surgical manipulations and closed-loop stimulation testing.

Stimulation of the CC was delivered using a pulse stimulator and a constant current stimulus isolation unit. Stimulation pulse trains were monophasic square waves that ranged in rate from 40 to 60 Hz, eliciting EMG in the form of compound muscle action potentials (CMAPs). CMAP EMG was recorded through hooked-wire pairs inserted independently with tip separation of approximately 10 mm oriented longitudinally within the CC and connected to the ILS communication adapter's analog EMG channel.

Stimulation of unilateral VF adduction from the ILS was tested with monophasic pulse trains 25 to 50 Hz, 0.1 to 0.8 ms pulse width, and 1.2 to 10 V. EMG blanking window length was varied systematically to determine blanking periods that effectively mitigated stimulation artifact without losing sensitivity to CC EMG between pulses. Stimulation effectiveness was assessed by visual inspection of recorded VF position at rest versus during pulse train delivery in relation to the glottic midline, as well as whether VF stimulation could be triggered by elicited CC contraction in a closed-loop manner, e.g., without having the VF stimulation contaminate the CC EMG and thereby cause a self-perpetuating positive feedback loop.

Stimulation with the ILS was also tested once in an awake animal at 3 weeks after implantation. A pulse train, e.g., 6.7 V amplitude, 0.6 ms pulse width, 50 Hz frequency, was delivered that had previously been shown to produce maximal VF adduction during anesthetized testing sessions in this animal, and the animal's reactions were noted and video recorded as stimulation was turned on and off multiple times. Stimulation delivery was confirmed by palpation of subtle stimulation-related movements from the larynx through the neck surface as well as stimulation delivery confirmation communicated from the implant back to the external hardware and computer interface.

### Methods—In Vivo BLS Testing

Three dogs were used for acute, i.e., nonimplanted testing of the BLS. Two of the dogs (dog No. 3 and dog No. 4) had a permanent nerve cuff electrode attached to the CC nerve supply on one side of the neck for periodic closed-loop testing as described previously. Dog No. 3 had intact laryngeal innervation and was tested at two timepoints. Dog No. 4 had bilateral synkinetic reinnervation and was tested at five timepoints between days 266 and 876 after bilateral recurrent laryngeal nerve transection and suture repair. Dog No. 5 had chronic Peterson-style electrodes implanted bilaterally within the VFs and chronic unilateral denervation when tested at a single timepoint 558 days after surgery.

The RLNs in Dog No. 4 were transected approximately 2.5 cm proximal to the inferior margin of the cricoid cartilage and suture-repaired with 3, 9-O Nylon sutures at the same time the nerve cuff electrode was implanted on the CC nerve supply, as discussed above. Referring now to FIG. 28B, a bipolar nerve cuff electrode 1400 with 1.5 mm internal diameters was placed on the left RLN approximately 2 cm proximal to the site of neurorrhaphy. Electrical connections to the cuff electrode 1400 was made by needle electrode 1408 insertion into the cuff electrode ECP 1406 implanted along the neck midline, allowing periodic stimulation of the RLN across months of recovery to track RLN reinnervation of the VFs, e.g., by elicited movements and recorded CMAPs.

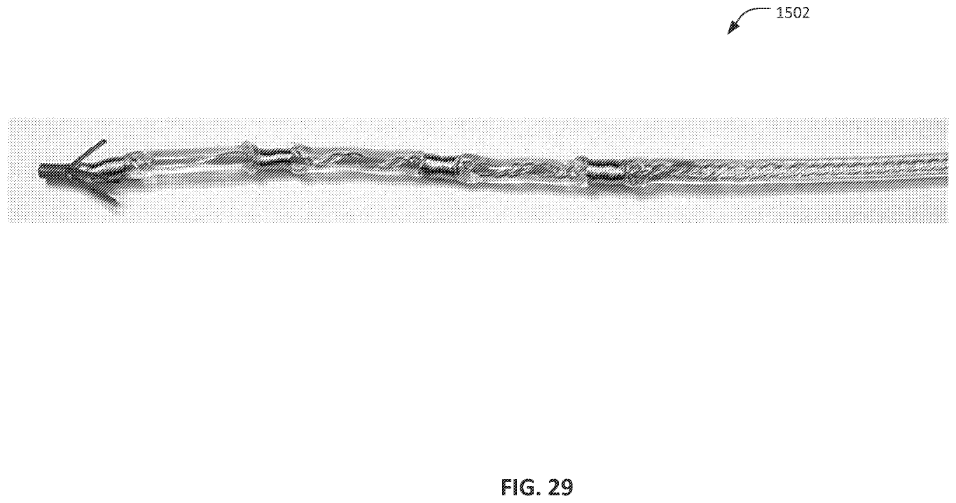

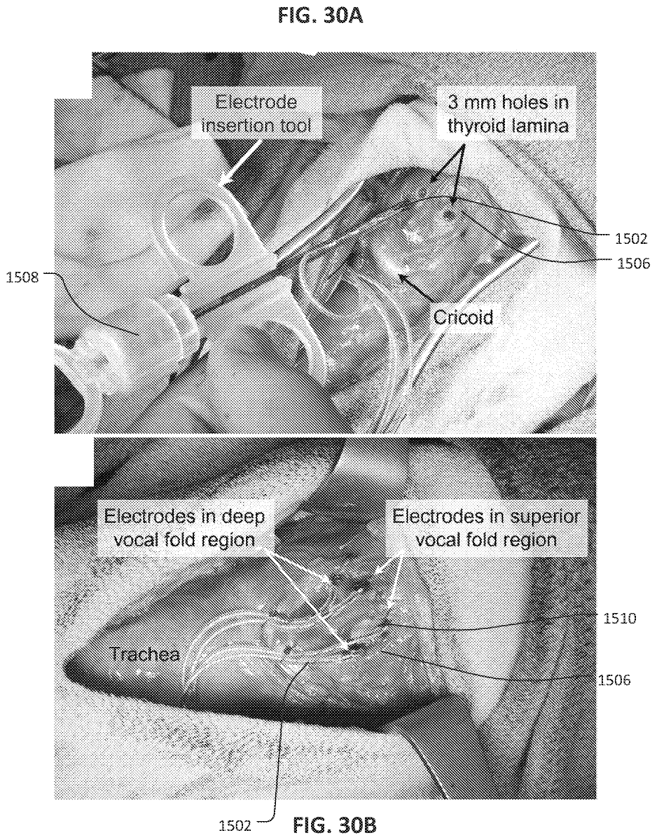

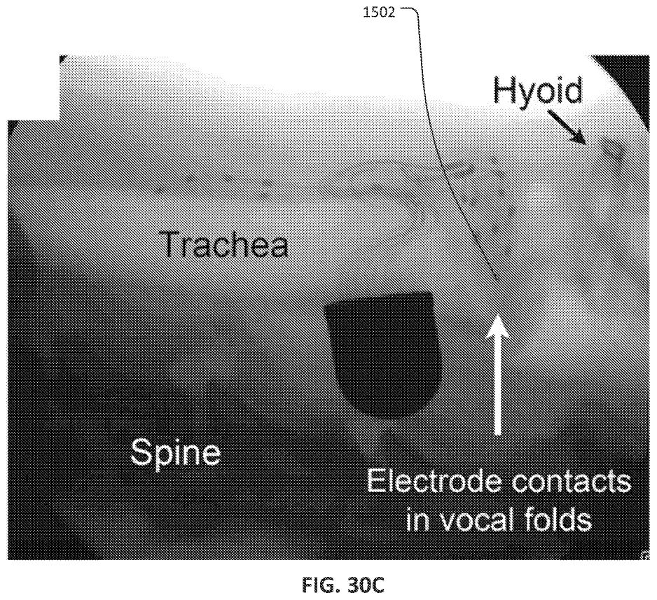

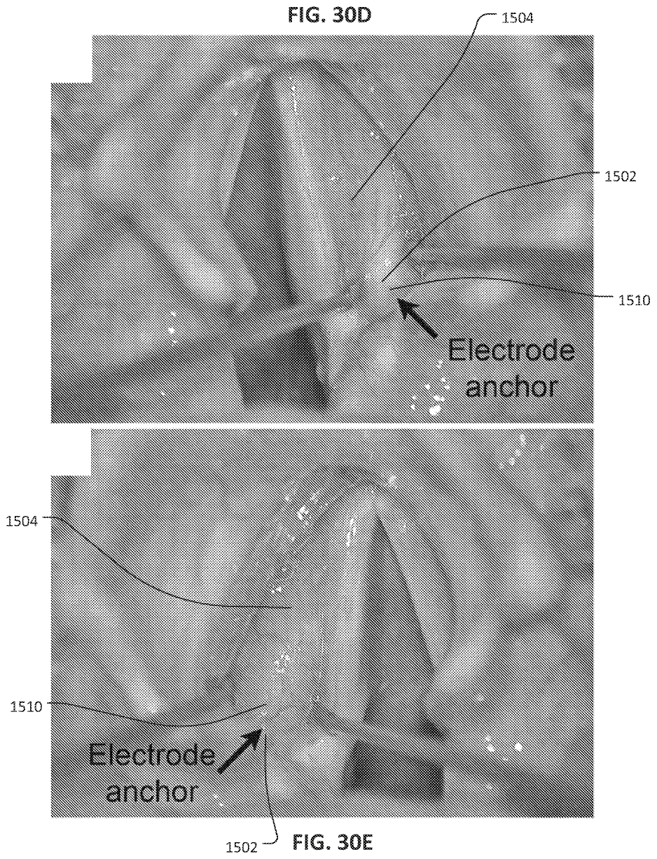

Referring now to FIGS. 29 and 30A-30F, multi-contact Peterson-style electrodes 1502 were implanted bilaterally, two multi-contact electrodes per side, in the superficial and deep paraglottic regions of the VFs 1504 (see FIGS. 30D and 30E) in dog No. 5. These electrodes 1502 are designed for intramuscular applications, with polymer barbs/tines at the distal tip to minimize movement after insertion and electrical contacts that encircle the electrode shaft to provide omnidirectional current delivery or EMG recording within a muscle. The electrodes were attached to a Myoelectric Implantable Recording Array, and surgical anesthesia and direct laryngoscopy were done as discussed above. Further, the Peterson electrodes 1502 were implanted into the VFs 1504 (see FIGS. 30D and 30E) in dog No. 5 with one pair inserted through 3 mm holes made in the thyroid lamina 1506 near the level of the superior VF surface using an insertion tool 1508, and a second pair was inserted deeper in the paraglottic VF on each side just under the inferior border of the thyroid cartilage. As shown in the non-limiting example of FIGS. 30D and 30E, tips of the electrodes 1502 had four to five polymer tines or anchors 1510 that helped anchor them in place after insertion, and which were visible through the VF mucosa. Specifically, the anchors 1510 were visible as resting just lateral to the vocal process of the arytenoid cartilage within each VF 1504. Each electrode 1502 had at least two contacts with the body of a VF 1504, which were effective for both EMG recording and VF adduction stimulation.

For testing with the BLS, the Peterson electrode leads were cut from the MIRA unit and attached to the stimulation and/or EMG recording leads of the BLS in the context of a terminal experiment 1.5 years after implantation. The right vocal fold was also denervated at the time of Peterson electrode implantation by transection and ligation of the RLN without repair, including removal of approximately 10 cm of RLN.

Further, Dog No. 5 underwent direct laryngoscopy with laryngeal EMG at days 28, 56, 83, 105, 140, 189, 210, 276, 553, and 558 after initial surgery. Visible twitching and small, e.g., 20-100 μV, ongoing EMG motor units recorded through hooked-wire electrode pairs inserted into the body of the right vocal fold, i.e., the thyroarytenoid and/or lateral cricoarytenoid region-also called TA/LCA herein, and PCA muscle were present by 4.5 months after RLN injury, indicating partial reinnervation from an unknown source. The right RLN was exposed at 6 months after initial injury, and electrical stimulation of the proximal RLN and right vagus nerves failed to cause VF movement or CMAPs, indicating that the RLN was not the source of laryngeal motor units. The proximal RLN was recut and ligated 2 cm more proximal to the original injury. The external branch of the superior laryngeal nerve (eSLN) was then transected at the cricothyroid muscle, and a 2 cm segment was removed. The right VF twitching and motor units persisted after eSLN transection, so a 2 cm segment of the internal branch of the right superior laryngeal nerve was also removed. The iSLN transection extinguished all ongoing movement and motor units, and the right side of the larynx remained still and electrically silent during the subsequent 6 months leading up to the terminal experiment with BLS testing. Therefore, the right side of the larynx had been RLN-denervated for 1.5 years and SLN-denervated for 1 year at the time of BLS stimulation.

Closed-loop stimulation of VF movement with the BLS was performed during acute experiments in dogs No. 3 through 5 for a combined total of eight testing days. Testing was performed with animals under general anesthesia and in suspension for direct laryngoscopy following methods similar to those described previously for ILS testing, although with slightly different stimulation parameters, e.g., 1-15 V, 0.1-1 ms pulses, 25-50 Hz. An overhead schematic view of the closed-loop BLS testing set-up 1600 is illustrated in FIG. 31. VF movements were elicited by multiple types of stimulation electrodes 1602 attached to the BLS 1604, including monopolar needles with approximately 5 mm uninsulated tips inserted transcervically, individual hooked-wires with 5 to 6 mm uninsulated tips inserted trans-orally, or chronically implanted Peterson electrodes. Closed-loop stimulation was triggered from hooked-wire EMG detected in the CC 1606 or vocal folds elicited using nerve-cuff electrodes 1608 placed on the spinal accessory or RLN, respectively. During the experiment, the CC 1606 contracted when the spinal accessory nerve was stimulated. The resulting EMG signal was detected through hook-wire electrodes 1610 and processed by the BLS 1604.

In addition to recording stimulated movement of the vocal folds, stimulation was combined with airflow-driven phonation to study the consequent modulation of phonatory parameters. Phonation was generated by placing a 2.1 mm hollow cannula 1612 into the trachea through the mouth and posterior glottis and passing heated and humidified air below the glottis while the VFs were either manually closed, e.g., with blunt instruments passed through the mouth, for cricothyroid stimulation testing or closed through electrical stimulation of laryngeal adductor muscles. A pressure probe 1614 with a 0.77 mm cable diameter was attached to the air cannula 1612 passing through the posterior glottis and used to measure tracheal pressure during phonation. All signals were digitized and recorded at 20 kHz/s/channel with hardware and software. Signals included tracheal pressure, overhead microphone 1616, current delivery to nerve cuff electrodes, high-speed video 1618 recording synchronization pulses, and the analog outputs of the BLS system, e.g., raw EMG, EMG envelope, blanking window, and stimulation delivery.

### Results

The ILS and BLS devices were each able to stimulate laryngeal muscle contraction in response to an experimentally elicited EMG trigger in multiple dogs, providing a successful proof of concept for these device designs. The ILS was implanted in two dogs without infection or electrode extrusion. ILS implants were tested across 17 testing sessions, and both units operated for more than one year. The BLS was tested outside of the body in three additional dogs across eight testing sessions. Multiple iterations of both the ILS and BLS were made across these 25 experiments. The BLS, which was developed subsequent to the ILS, has some improved features, including, a wider stimulation voltage range, utilizing charge-balanced pulses rather than monopolar pulses, and improvements in stimulation artifact blanking through hardware and software modifications. The improved artifact rejection made it possible to use EMG detected from one side of the larynx to trigger stimulation of the contralateral VF without self-perpetuated positive feedback.

ILS implants were tested across survival periods of 238 and 728 days in dogs 1 and 2, respectively. The implant in dog No. 1 was verified as functional for laryngeal stimulation at days 0, 21, 101, 119, and 238, at which point the animal was euthanized and the implant was removed. The implant in dog No. 2 was verified as functinal at days 0, 13, 21 (awake), 24, 49, 129, 139, 147, 266, 427, and 504. When tested at day 665, stimulation at what had been supramaximal voltage level, i.e., greater than 4.1 V, 7 ms pulse width, tested up to 9 V, did not produce visible laryngeal movements. At the time of euthanasia (day 728), the implant electrode was still in position on the epimysial surface above the LCA, and possibly lateral TA, muscle surface.

Examples of unilateral VF adduction stimulated on the right and left sides in dogs 1 and 2 are shown in FIGS. 32A-32D, 2 at days 238 and 266, respectively. Adduction that reached or exceeded the midline was achieved at the time of implantation as well as later dates with stimulation parameters of ≥4.1 V, 0.7 ms pulse width, 50 Hz. Fused contraction of laryngeal adduction was observed with stimulation rates of 40 Hz or higher in normally innervated VFs, which was shown dogs 1-3, and one VF in dog No. 5. In some experiments, contraction thresholds were measured with ILS stimulation pulse rate at 50 Hz and pulse width held constant at 0.7 ms while varying stimulation amplitude, e.g., 1.9, 3, 4.1, 5.5, 6.7, 7.8, and 9 V, and with varying pulse widths, e.g., 0.1, 0.2, 0.3, 0.5, 0.7, 0.8 ms, at a constant amplitude of 6.7 V. Neurally intact VFs had relatively low thresholds for movement, typically twitching at 3 V at 0.7 ms pulses. The difference between threshold and maximal stimulation was approximately 1 V in most cases, suggesting that stimulation was affecting axons entering target muscles rather than muscle cells directly.

Successful closed-loop stimulation of VF adduction was achieved in both animals with ILS implants through activation of the CC muscle and triggering from the CMAP EMG envelope. Chronically implanted cuff electrodes to the CC nerve supply were electrically accessed by temporary insertion of needle electrodes into the ECPs beneath the skin surface, and CC contraction thresholds were typically <0.25 mA (300 μs pulse width, 60 Hz). Closed-loop stimulation artifact in the CC EMG recording, stemming from the VF stimulation location, was sometimes strong enough to maintain suprathreshold CC EMG levels even when the nerve cuff stimulation was stopped, generating a self-perpetuating positive feedback loop. Blanking of the CC EMG during each VF stimulation pulse effectively mitigated stimulation artifact, enabling stimulation to occur only when the CC was experimentally driven through the nerve cuff.

Dog No. 2 was tested awake at three weeks after ILS implantation with the goal of observing reactions to stimulated VF adduction that might indicate pain or discomfort. Communication between the internal and external system hardware indicated the successful delivery of VF stimulation, and there was palpable movement from the larynx as the stimulation was turned on/off. Stimulation amplitudes were tested at more than twice the level needed for maximal VF adduction, which was determined during anesthetized recordings, i.e., up to 6.7 V, 50 Hz, 0.6 ms pulse width, yet the dog showed no reaction to stimulation aside from swallowing once at the initiation of the first pulse train in the test series.

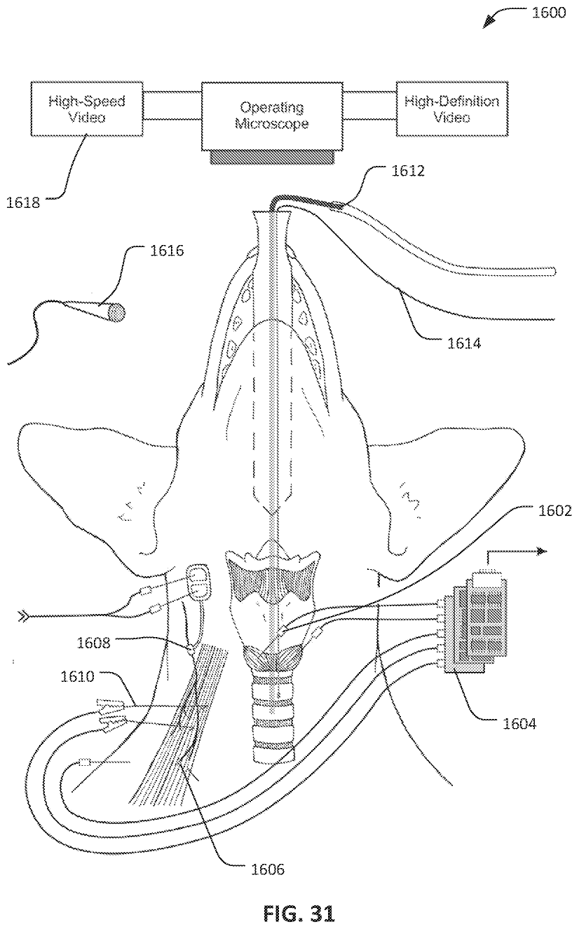

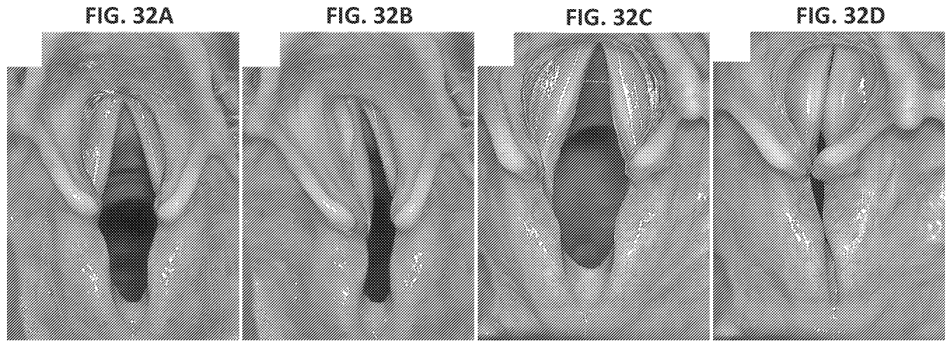

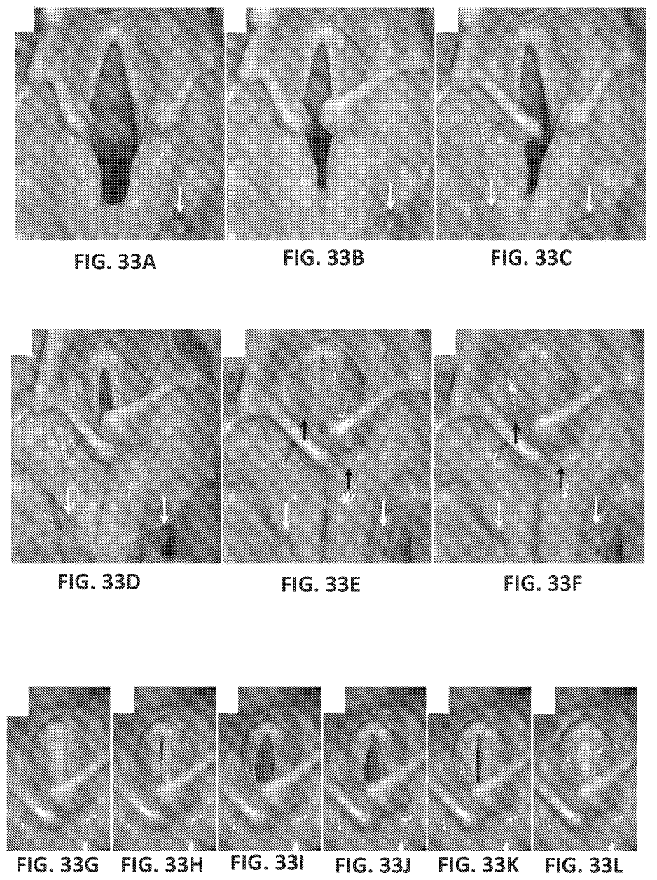

Successful closed-loop stimulation of VF adduction was achieved in dogs 3 and 4 with the BLS system by triggering from the CC EMG envelope, as was done in dogs 1 and 2 with the ILS system. Stimulation in the larynx was performed with needle and hooked-wire electrodes placed bilaterally, enabling glottic closure that produced phonation when tracheal air pressure was created (see Methods). To close the posterior glottis and achieve glottic competence for effective airstream valving, the interarytenoid (IA) muscles needed to contract. Unlike humans, dogs have a midline interarytenoid cartilage onto which paired right and left IA muscles attach. Although the IA blends with the more dorsally located unpaired ventricularis muscle spanning the midline, it is possible to stimulate right and left IA muscles independently, as illustrated in FIGS. 33B and 33C, respectively, in comparison to the glottic configuration at rest illustrated in FIG. 33A. Further, bilateral stimulation of the IA closes the posterior glottis, yet leaves an anterior glottic gap, as depicted in FIG. 33D, that requires TA/LCA stimulation for complete closure. Bilateral stimulation of the IA and TA/LCA muscles through the bipolar stimulation channel of the BLS, i.e., passing current across the 2 halves of the larynx, generated adequate closure for phonation, as illustrated in FIGS. 33E and 33F.

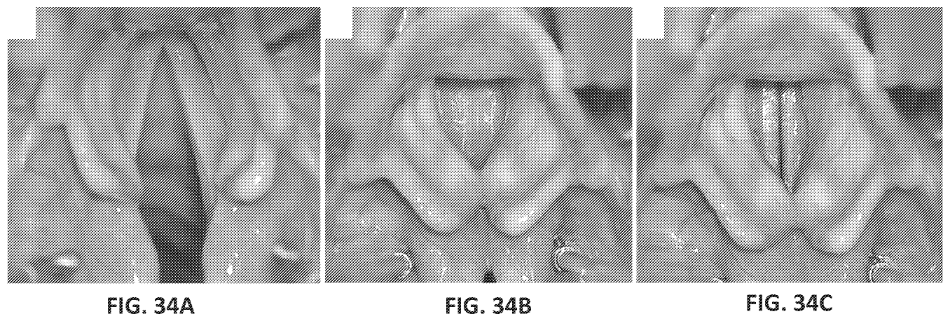

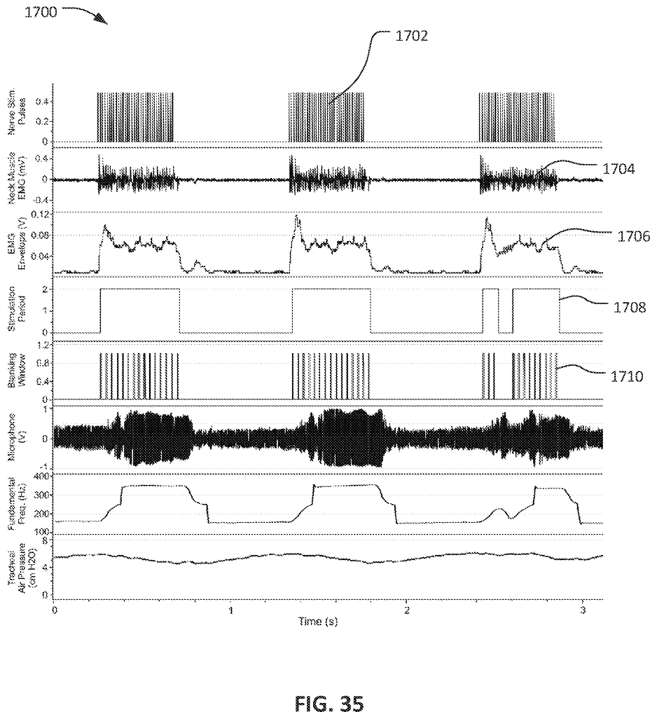

Referring now to FIGS. 33G-33L, high-speed video revealed a normal-looking phonatory pattern with symmetrical VF excursions during BLS testing. Specifically, FIGS. 33G-33L illustrate a sequence of high-speed video frames, each separated 1.5 ms apart, that demonstrate a typical cycle of VF vibration at 133 Hz. Bilateral stimulation of the CT muscle was also performed during manual closure of the arytenoids in dog, as illustrated in FIGS. 34A-34C, stretching the VFs and increasing phonation fundamental frequency. Specifically, the posterior glottis was manually closed using blunt cupped forceps, and phonation was initiated with tracheal air pressure delivered through a cannula, as discussed above. VF stretching occurred with each bilateral cricothyroid (CT) contraction (see FIG. 34C). Referring now to FIG. 35, recorded signals 1700 representing BLS functions and physiological signals show three representative stimulations among an ongoing series. The top signal 1702 shows when the nerve supply of the cleidocephalicus (CC) was stimulated at 60 Hz for approximately 400 ms each second. The second and third signals 1704, 1706 show the resulting EMG signals, and EMG envelope recorded from the CC, and the fourth signal 1708 shows when the envelope exceeded the triggering threshold to generate stimulation periods. The series of blanking windows in the fifth signal 1710 indicate when CT muscle stimulation pulses were delivered, at 40 Hz, and when the recorded EMG was held at zero, i.e., blanked. Phonation became louder and higher in frequency with each CT contraction, with fundamental frequency increasing from 160 to about 365 Hz. Note that CC EMG briefly dipped below activation threshold in the third pulse train, causing a momentary cessation of CT stimulation. Lowering the activation threshold prevented such cutouts in subsequent trials.

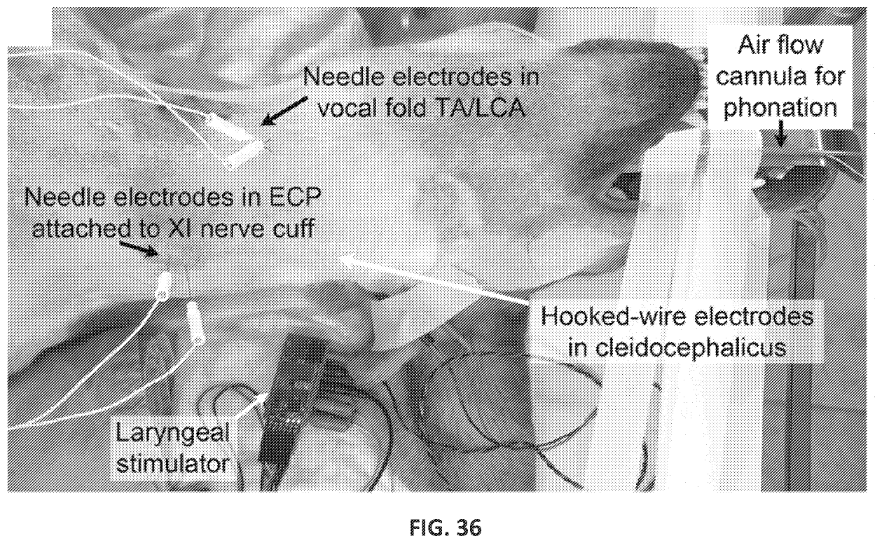

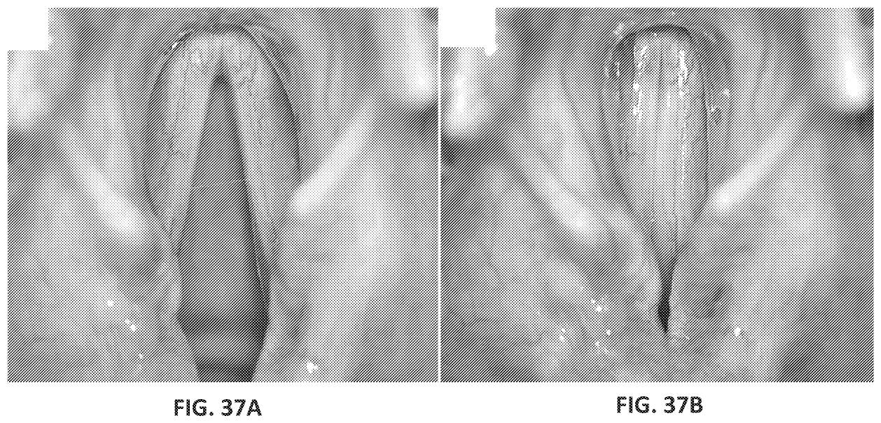

Further, dog No. 4 was tested five times between approximately nine and 29 months after bilateral RLN transection and suture repair. Bilateral synkinetic reinnervation was present based on common EMG activity observed across TA/LCA and PCA recording locations during inspiration and glottic closure reflex and an absence of slight VF inspiratory abduction typically observed under Propofol anesthesia. As illustrated in FIG. 36, bilateral VF adduction was stimulable with the BLS through needle electrodes placed transcervically or stimulation of the nerve cuff electrodes chronically implanted on the RLNs. Stimulations parameters for eliciting threshold and maximal adduction were similar for the synkinetically reinnervated VFs of dog No. 4 as observed with intact nerve supplies in dogs 1 through 3. For example, needle electrode stimulation threshold was 1.9 V, with 0.5 ms pulse width and 40 Hz, with sustained contractions at 4.1 V in dog No. 4. Another example of closed-loop testing of glottic closure in dog No. 4 is presented in FIGS. 37A and 37B, which illustrate the anterior glottis in an open position and a closed position, respectively, as a result of TA/LCA stimulation.

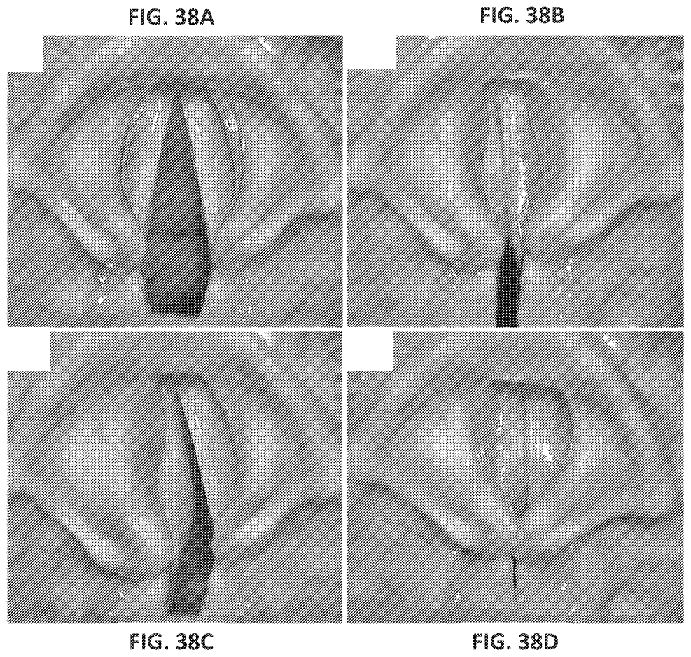

In addition, dog No. 5 had two Peterson electrodes implanted in each VF for 558 days, with chronic RLN and SLN denervation of the right VF. Stimulation through either electrode on both sides of the larynx was effective for eliciting VF adduction. As expected, the denervated VF required higher stimulation levels than the intact VF. For example, movement threshold for the intact VF was 0.28 mA, with 0.5 ms pulse width and 60 Hz, whereas threshold for the denervated VF was 0.8 mA. Maximal contraction of denervated VF adduction was not achieved with typical pulse widths of 0.5 ms at currents up to 5 mA (60 Hz), but increasing pulse duration to 1 ms enabled maximal contraction to be reached by approximately 3.5 mA (1.1 mA for the intact VF). Therefore, stimulation strength needed to elicit minimal and maximal contractions of the denervated VF were approximately 3 times greater than what was required for the intact VF in this animal. As illustrated in FIGS. 38A and 38B, bilateral stimulation through the chronically implanted electrodes produced complete anterior glottic closure but left an approximately 2 mm gap in the posterior glottis due to the lack of IA stimulation.

EMG activity from the CC was used to trigger closed-loop stimulation of the VFs using the BLS in dog No. 5. Closed-loop stimulation was also achieved using CMAP EMG detected from the healthy side of the larynx, elicited by RLN stimulation, to trigger stimulated adduction of the paralyzed VF. When the left, i.e., intact, RLN was stimulated, e.g. at 0.25 mA, 0.1 ms pulse width, 60 Hz, in conjunction with the right, i.e., denervated VF using the Peterson electrode in either the superficial or deep para-glottic region, it closed the anterior and posterior glottis sufficiently for phonation, as illustrated in FIGS. 38C and 38D. In the illustrated non-limiting example, the sufficient glottic closure reflects right VF stimulation at 3.2 V, 1 ms pulse width, 33 Hz. In this scenario, the stimulated VF was near the EMG recording location for device triggering, i.e., the contralateral VF, yet the BLS system was able to effectively blank out the stimulation artifact using a 4 ms blanking window and avoid self-perpetuating positive feedback loops.

### Discussion

Unilateral VF paralysis, via the RLN, can cause a hoarse, strained, breathy voice that has reduced loudness and pitch range and requires increased effort to produce due to aerodynamic incompetence of the glottal valve. Bilateral RLN paralysis can also compromise breathing and swallowing. Surgical procedures for unilateral paralysis include varied forms of static reconstruction, including injection medialization of the immobile VF through paraglottic injection of bulking materials, transcervical medialization through rear-ranging the cartilaginous framework, repositioning the arytenoid, and neural anastomosis. Although these techniques can provide good outcomes, there is no dynamic restoration of motion, and the results can be inconsistent in the most experienced hands. Treatments for bilateral VF paralysis typically require even more challenging decision making due to the potential simultaneous compromise of airway, swallowing, and vocal function. Therefore, dynamic and reversible FES options for restoring glottic competence in unilateral and bilateral laryngeal denervation is desirable if FES can safely restore function that equals or exceeds static solutions.

Researchers and clinicians have long recognized the potential for FES to restore laryngeal function, motivated by the natural symmetry in VF movement useful for treating unilateral palsy and the availability of coincident contractions from other neck muscles that can be used as a proxy EMG source for triggering laryngeal function in bilateral palsy. Multiple studies have demonstrated effective and well-tolerated chronic laryngeal FES in awake animals or individuals using preset stimulation patterns, i.e., open-loop, under conditions of normal innervation or synkinetic reinnervation. Most importantly, effective stimulation of the posterior cricoarytenoid (PCA) for VF abduction in human patients with bilateral VF paralysis has been well tolerated in clinical trials conducted in the United States (22 n=5 stimulated; 2-7 V, 1 ms pulses, 30-40 Hz) and in Europe (21 n=7 stimulated; 1.2-4 mA, 0.2-2.1 ms pulses, 28-49 Hz). An inclusion criterion in both human trials was evidence of at least partial reinnervation of the PCA receiving FES. Therefore, all long-term testing of laryngeal FES devices to date has involved predetermined stimulation patterns at current levels appropriate for innervated muscle. However, dynamic restoration of abductor and adductor glottic control will require flexible triggering from physiological signals, i.e., closed-loop, at stimulation levels adequate for the innervated and denervated muscle to serve the widest clinical population. The custom-built devices and canine test conditions presented in the present example embody a step toward achieving such a closed-loop laryngeal FES system.

As discussed above, effective VF adduction was stimulated in four dogs with intact laryngeal innervation, i.e., seven VFs, one dog with bilateral synkinetic reinnervation, i.e., two VFs, and one dog with unilateral chronic denervation, i.e., one VF. All of the innervated or reinnervated VFs contracted to the midline or beyond at relatively low stimulation levels, e. g., ≥4.1 V, ≥0.5 ms pulse width, ≥50 Hz, that prior studies suggest would be tolerated in awake animals or individuals. Stimulation of the chronically denervated VF, e.g., >1 year post RLN and SLN transection, required approximately three times the stimulation strength, e.g., 3.2 V, 1 ms pulse width, 33 Hz, compared to innervated or reinnervated VFs, which is consistent with the need to stimulate a large volume of individual muscle cells in denervated muscle rather than relatively few axons passing through the stimulation field. Nevertheless, this higher stimulation requirement was likely still within a tolerable range. For example, comparable stimulation, e.g., 6.7 V, 0.6 ms pulses, 50 Hz, performed in one of the two dogs receiving the ILS implant while they were fully awake caused no indications of discomfort during multiple, sustained activations, i.e., several multisecond-long pulse trains. Thus, the present results suggest that LFES, even in cases of longstanding denervation, does not require damaging or painful electrical currents. In addition, it was demonstrated that FES could be triggered from one innervated VF while the system delivered stimulation strong enough to adduct the contralateral, chronically denervated VF without generating a self-perpetuating positive feedback loop. This successful mitigation of stimulation artifact through EMG blanking is a hopeful indication that closed-loop FES can be achieved between healthy and paralyzed sides of the larynx even after longstanding paralysis, e.g., 1.5 years of denervation in the present example.

The ILS and BLS implants had electrodes composed of polymer-insulated surgical stainless steel. The bipolar paddle-shaped electrodes attached to the ILS were implanted for 238 and 728 days, in dogs 1 and 2, respectively, and the four Peterson electrodes were implanted for 558 days in dog No. 5. At the end of the experiment, none of these electrodes were corroded or showed signs of tissue interaction aside from anticipated encapsulation. The same general materials and design of these paddle and Peterson-style electrodes were chronically implanted in the same locations in prior studies of laryngeal FES, e.g., 61 for the epimysial paddle, and 59 for the intra-muscular Peterson-style, and both reports indicated excellent performance of these electrode designs. Further, effective VF adduction was stimulable with either the relatively superficial or more lateral and deep implantation of Peterson electrodes on either the innervated or denervated side in dog No. 5, suggesting that precise placement is not necessarily required when using a bipolar arrangement with 6 to 7 mm interelectrode distance, In addition, although the more superficial Peterson electrodes were near the mucosal surface, with visible anchoring tines, the electrodes did not move or extrude during the 1.5-year survival period.

Additional work is needed to identify optimal electrode designs and materials for long-term, frequent stimulation within the highly mobile tissue of the larynx. Both sides of the electrode/tissue interface can experience damage during FES, but strategies such as delivering charge-balanced pulses, e.g., those utilized by the present BLS implant, and using relatively large contact areas composed of materials like iridium oxide can minimize toxic pH changes and oxidation-reduction reactions. Furthermore, fabricating electrodes from biologically inert conductive polymers would allow them to flex, stretch, and have similar softness as surrounding muscle and nerve for optimal longevity and tissue compatibility.

Correspondingly, EMG-triggered stimulation of VF adduction was able to produce phonation when glottic airflow was generated experimentally, and stimulation of the CT substantially increased ongoing phonation. This reflects an important proof of concept for functional closed-loop stimulation of VF adduction and phonation modulation using an FES implant. In conclusion, the present example demonstrates dynamic stimulation of adduction in healthy, reinnervated, and chronically denervated VFs using custom, implantable hardware at stimulation levels tolerable during awake testing. EMG-based, closed-loop activation of VF adduction was possible due to hardware and software mitigation of stimulation artifact, working even when the recording and stimulation locations were both within the larynx.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks, e.g., compact disks and digital video disks, magnetic cassettes, memory cards or sticks, random access memories (RAMs), read only memories (ROMs), and the like.

As used in the claims, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present invention has been described in terms of one or more preferred embodiments, and it should be appreciated that many equivalents, alternatives, variations, and modifications, aside from those expressly stated, are possible and within the scope of the invention.

What is claimed is:

1. A system for physiological sensing and laryngeal stimulation, comprising:
at least one sensor configured to be secured to a subject to record electromyographic (EMG) signals of the subject;
a processor coupled to the at least one sensor to receive the recorded EMG signals and generate a stimulation signal configured to stimulate at least one vocal fold of the subject to treat glottic insufficiency, improve airway protection, or both; and
an electrode configured to be secured to the subject to deliver the stimulation signal to an area of interest in the subject to stimulate the at least one vocal fold of the subject to treat glottic insufficiency, improve airway protection, or both.

2. The system of claim 1, further comprising a controller to monitor the EMG signals recorded by the at least one sensor and execute one or more control processes to regulate generation of the stimulation signal.

3. The system of claim 1, wherein the at least one sensor and the electrode are coupled to a receiver, and wherein the receiver is coupled to a transmitter via a wireless communication interface.

4. The system of claim 3, wherein the transmitter includes a first coil and the receiver includes a second coil, and wherein the first coil of the transmitter is inductively coupled to the second coil of the receiver to provide wireless communication and power between the transmitter and the receiver.

5. The system of claim 3, wherein the receiver is implanted subcutaneously within at least one of a neck, a head, a thorax, or an abdomen of the subject, and wherein the transmitter is located external to the subject.

6. The system of claim 1, wherein the electrode is one of a plurality of electrodes, and wherein the plurality of electrodes are placed between a thyroarytenoid muscle and a lateral cricoarytenoid muscle within the subject.

7. The system of claim 1, wherein the electrode is a bipolar or tripolar epimysial electrode.

8. The system of claim 1, wherein the electrode includes a first plurality of disc contacts disposed on a first paddle and a second plurality of disc contacts disposed on a second paddle.

9. The system of claim 8, wherein the electrode is bifurcated into a first arm and a second arm such that the first paddle is coupled to a first side of the first arm, the second paddle is coupled to a second side of the first arm, and a third paddle including a third plurality of disc contacts is coupled to the second arm.

10. A system for stimulating at least one vocal fold in a subject, comprising:
at least one sensor for recording electromyography (EMG) signals of the subject;
a processor coupled to the at least one sensor, the processor to determine if the EMG signals correspond to intended movement of the at least one vocal fold of the subject;
a generator to selectively generate a stimulation signal if the analyzed EMG signals are determined to correspond to the intended movement of the at least one vocal fold of the subject; and
an electrode to provide the generated stimulation signal to a region of interest in the subject to elicit a movement of the at least one vocal fold of the subject corresponding to the intended movement of the at least one vocal fold of the subject.

11. The system of claim 10, further comprising a battery pack that provides power to the processor and the generator, and wherein the battery pack and the generator are configured to be implanted under the subject's skin.

12. The system of claim 10, wherein the at least one sensor and the electrode are coupled to a receiver, and wherein the receiver is coupled to a transmitter via a wireless communication interface.

13. The system of claim 12, wherein the wireless communication interface includes a plurality of light-emitting diodes and a plurality of phototransistors that provide communication of serial data between the receiver and the transmitter.

14. The system of claim 12, wherein the processor is disposed on the receiver, and wherein the EMG signals recorded by the at least one sensor are analyzed by the processor, transmitted from the receiver to the transmitter via the wireless communication interface, and further analy zed by a monitoring system coupled to the transmitter.

15. A method of stimulating a larynx of a subject, comprising:
detecting, with at least one sensor coupled to the subject, electromyography (EMG) signals corresponding to an intended contraction of at least one vocal fold of the subject;
analyzing the EMG signals to determine a stimulation signal for contracting the at least one vocal fold; and
delivering the stimulation signal to an area of interest in the subject to stimulate the at least one vocal fold of the subject to elicit a contraction of the at least one vocal fold of the subject corresponding to the intended contraction of the at least one vocal fold of the subject indicated in the EMG signals.

16. The method of claim 15, further comprising implanting an implantable device within at least one of a neck, a head, a thorax, or an abdomen of the subject, the implantable device including a processor to perform the analyzing of the EMG signals.

17. The method of claim 15, further comprising:
performing a blunt dissection of the at least one vocal fold of the subject to create a cavity; and
inserting an electrode into the cavity such that the electrode is positioned between a thyroarytenoid muscle and a lateral cricoarytenoid muscle within the subject.

18. The method of claim 15, further comprising selectively generating the stimulation signal if an amplitude of the EMG signals reaches an activation threshold and remains greater than a termination threshold value.

19. The method of claim 15, further comprising selectively generating the stimulation signal with a strength proportional to an amplitude of the EMG signals.

20. The method of claim 15, further comprising selectively terminating the stimulation signal if an amplitude of the EMG signals is less than a termination threshold value.

21. The method of claim 15, further comprising selectively generating the stimulation signal using a remote control.
